SEQUENCE LISTING (2 sequences)

SEQ ID NO: 1 - protein, 35 aa, Escherichia coli
  MISC_FEATURE (13)..(13): Xaa is 2-(2-aminomethyl-oxazol-4-yl)-thiazole- 4-carboxylic acid
VGIGGGGGGGGGXGGQGGXGXSNXGGNGXGXGSHI

SEQ ID NO: 2 - protein, 7 aa, Artificial sequence
  source 1..7: /note= “Description of artificial sequence Synthetic sequence”
QGAXGGA

(19) United States
(12) Patent Application Publication
(10) Pub. No.: US 2008/0234132 A1
(43) Pub. Date: Sep. 25, 2008

(54) Microcin B17 Analogs And Methods For Their Preparation And Use

(76) Inventors: Laurence Coquin, Nice (FR); Fabrice Jourdan, Bristol Avon (GB); Olivier Pierrat, Norwich Norfolk (GB); David John Lowe, Harleston Norfolk (GB); Anthony Maxwell, Attleborough Norfolk (GB); Christopher John Pickett, Upton Norfolk (GB); Melisa Wall, Queensland (AU)

Correspondence Address: NIXON & VANDERHYE, PC, 901 NORTH GLEBE ROAD, 11TH FLOOR, ARLINGTON, VA, 22203, US

(21) Appl. No.: 11/667,385

(22) PCT Filed: Nov. 18, 2005
(86) PCT No.: PCT/GB05/04458

§ 371 Date: Nov. 19, 2007

(30) Foreign Application Priority Data
Nov. 19, 2004 (GB) 0425532.9
Jul. 1, 2005 (GB) 0513546.2

(51) Int. Cl. A01N 43/36; C07K 7/06; A61K 38/08; A61P 35/00; A01P 1/00; A01P 21/00; A61P 31/04; C12N 9/99

(52) U.S. Cl. 504/287; 530/329; 514/16; 435/184

(57) ABSTRACT

The present invention pertains to synthetic analogues of microcin B17 component units, methods of making and using these analogues, including, for example, as inhibitors of DNA gyrase. More particularly, the present invention pertains to compounds of the Formula wherein: W is independently: —H or a peptide group; Z is independently: —OH or a peptide group; wherein each peptide group, if present, is: an amino acid group and comprises exactly one amino acid, or: a poly(amino acid) group and comprises two or more amino acids; R^{N3} is independently: —H, C₁₋₆alkyl, C₂₋₆alkenyl, C₃₋₆cycloalkyl, or C₃₋₆cycloalkenyl, C₆₋₁₄-carboaryl, C₅₋₁₄heteroaryl, C₆₋₁₄carboaryl-C₁₋₆alkyl, C₅₋₁₄heteroaryl-C₁₋₆alkyl, and is optionally substituted; the circle represents a mono-heterocycle or a bis-heterocycle, wherein the heterocycle, or each of the two heterocycles, is a five membered ring having at least a first ring heteroatom that is N, and optionally a second ring heteroatom that is selected from N, O, and S; and the heterocycle, or each of the two heterocycles, is optionally substituted with one or more substituents; and pharmaceutically acceptable salts, amides, esters, solvates, and hydrates thereof. The present invention also pertains to uses of such compounds, for example, to inhibit DNA Gyrase activity in a cell and in methods of therapy, for example, to treat a disease or condition that is ameliorated by the inhibition of DNA Gyrase, such as a bacterial infection, cancer, etc.; as a herbicide; as a microbicide; as a bactericide; etc.

### RELATED APPLICATIONS

This application is related to: United Kingdom patent application number 0425532.9 filed 19 Nov. 2004, and United Kingdom patent application number 0513546.2 filed 1 Jul. 2005; the contents of each of which are incorporated herein by reference in their entirety.

### FIELD OF THE INVENTION

This invention pertains to water soluble synthetic analogues of microcin B17 component units, methods of making and using these analogues, including, for example, as inhibitors of DNA gyrase.

### BACKGROUND TO THE INVENTION

A number of publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Throughout this specification, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in this specification, the singular forms “a,” “an,” and “the” include plural referents unless the context clearly dictates otherwise. Thus, for example, reference to “a pharmaceutical carrier” includes mixtures of two or more such carriers, and the like.

DNA gyrase is the only bacterial enzyme that introduces negative supercoils into relaxed closed circular DNA, to maintain an appropriate degree of negative supercoiling to allow the replication of DNA. Inhibition of DNA gyrase by some antibiotics leads to the generation of increasing positive supercoiling, rapidly generating resistance to further movement of the DNA replication fork. Accordingly, there is a need for improved inhibitors of DNA gyrase for use as potent and specific antibiotics.

Microcin B17 (MccB17) is a peptide antibiotic that inhibits DNA replication in Enterobacteriaceae. MccB17 blocks DNA gyrase by trapping an enzyme-cleaved-DNA complex. Thus, the mode of action of this peptide antibiotic resembles that of quinolones and a variety of antitumour drugs currently used in cancer chemotherapy. The mode of action of MccB17 has not yet been fully elucidated. MccB17 is a 3.1 kDa post-translationally-modified peptide that traps DNA gyrase and cleaved DNA in a covalent complex, which acts as a barrier to DNA polymerase, thereby inhibiting DNA replication. Genetic mutations in position IIₐ (FIG. 1) have been shown to be involved in the activity of MccB17.

Microcin B17 (MccB17) is however poorly soluble in water and this severely limits its potential application as a drug. However, preliminary results have shown that water soluble component parts of MccB17 inhibit the supercoiling reaction of DNA gyrase.

In this disclosure, the inventors provide novel, hydrophilic analogues of component units of MccB17 as well as methods of making and using these compounds which should extend the utility of MccB17 and provide a lead for further antibiotic drug developments.

### SUMMARY OF THE INVENTION

The present invention pertains to novel (preferably hydrophilic) analogues of component units of MccB17 (as described herein) as well as methods of making and using these compounds, which should extend the utility of MccB17 and provide a lead for further antibiotic drug developments. Also described herein is a total synthesis of MccB17 unit I and II (see, e.g., FIG. 1) analogues for subsequent insertion in small peptidic structures.

One aspect of the present invention pertains to novel (preferably hydrophilic) analogues of MccB17 component units, as described herein.

Another aspect of the present invention pertains to methods of making and using these analogues.

Another aspect of the present invention pertains to methods for developing further analogues of MccB17 and its component units.

Further objects and advantages of this invention will become apparent from a review of the full disclosure.

### BRIEF DESCRIPTION OF THE DRAWINGS

FIG. 1 provides a representation of Microcin B17 (MccB17).

FIG. 2 provides a representation of some MccB17 unit I (mono-heterocyclic) component analogues and unit II (bis-heterocyclic) component analogue precursors.

FIG. 3 provides a schematic of solid phase synthesis (Merrifield resin) for Peptide 1: Gly (x2), 7a, Gly, Ala and Glu are assembled to yield 8 following the cycle as follows: deprotection of the N-Boc protective group with a 25% TFA solution in DCM; coupling of an N-protected amino acid using a BOP/HOBt in NMP/DCM (1:1) activation method; cleavage of the peptide using TFMSA/TFA anisole/EDT and purification by ether precipitation.

FIG. 4 provides a schematic of synthesis of compounds G, J, L and M: i) NaBH₄ in MeOH, 0° C., ii) P(Ph)₃, DEAD (diethylazidocarboxylate), diphenylphosphorylazide, −20° C. followed by addition of P(Ph)₃, water, 45° C.

FIG. 5 provides a supercoiling test in which DNA supercoiling reactions were carried out as described by Pierrat & Maxwell (2003) except that the gyrase, referred to herein as [A₂B₂], was 13.2 nM; reactions were incubated for up to 4 h, and the reactions analysed by electrophoresis: Lane 1: without A₂B₂ (no enzyme); Lane 2: DMSO (2%); Lane 3: MccB17 25 μM; Lane 4, 5, 6: Peptide 1 respectively at 200 μM, 100 μM and 50 μM.

FIG. 6 provides a DNA cleavage test in which [A₂B₂]=30 nM, and wherein reactions were incubated up to 1 h: Lanes 1, 2, and 3: 30 minutes; Lanes 4, 5 and 6: 1 hour; Lanes 1 & 3: no enzyme; Lanes 2 & 4: DMSO (2%); Lanes 3 & 6: Peptide 1 at 100 μM.

FIG. 7 provides a synthetic scheme for Peptide 1.

FIG. 8 provides a synthetic scheme for Peptide 2.

FIG. 9 provides a graphic representation of the antimicrobial efficacy of Peptide 1 as compared to Microcin B17 (a plot of diameter (mm) versus concentration (μm)).

FIG. 10 provides a demonstration of bacterial grown inhibition on a growth medium as follows: IA: MccB17, 50 μM; IB: MccB17, 25 μM; IC: MccB17, 10 μM; ID: MccB17, 5 μM; IE: MccB17, 2.5 μM; IIA: Peptide 1, 50 μM; IIB: Peptide 1, 125 μM; IIC: Peptide 1, 254 μM; IID: Peptide 1, 508 μM.

FIG. 11 Peptide 1-induced DNA unwinding was examined using a DNA topoisomerase I-based assay: Lane 1: DMSO (2%); Lanes 2, 3, 4: MccB17 at 1 μM, 10 μM, 50 μM; Lanes 6, 7, 8: Ethidium bromide at 0.5 μM, 2 μM, 5 μM; Lanes 10, 11, 12: Ciprofloxacin at 1 μM, 10 μM, 50 μM; Lane 14: DMSO (2%); Lanes 15, 16, 17: Peptide 1 at 20 μM, 50 μM, 100 μM. Lanes 1, 14: No drug: Gaussian distribution; Lanes 2-5: +MccB17: no change, no visible intercalative property; Lanes 6-9: +Ethidium Bromide (EtBr): strong intercalative agent; Lanes 10-13: +CFX: 100-fold weaker intercalative agent than EtBr; Lanes 15-19: +Peptide 1: no significant change but Peptide 1 may inhibit the relaxation reaction by topo I at high concentration.

FIG. 12 bacterial growth inhibition plates were incubated at 37° C. overnight and growth inhibition was qualitatively analysed by measuring the diameter of each halo. Mutant bacteria are resistant to MccB17 through a known mutation (W751R DNA gyrase B subunit). Bacteria bearing this mutation are also resistant to Peptide 1, suggesting that both Peptide 1 and MccB17 have a common binding site on DNA gyrase.

FIG. 13 supercoil inhibitory assay using Peptide 2: Lane 1: without A₂B₂ (no enzyme); Lane 2: DMSO 10%; Lane 3: Peptide 1 (100 μM); Lane 4: Peptide 2 (100 μM); Lane 5: Peptide 2 (200 μM); Lane 6: Peptide 2 (50 μM).

FIG. 14 provides a representation of Peptide 3, Peptide 4, and Peptide 5.

FIG. 15 provides a synthetic scheme for analogue C.

FIG. 16 shows the relaxation assay gels for Peptides 1, 2, and 5.

FIG. 17 is a graph of relative ATPase rate (%) versus concentration of inhibitor (μM) for Peptides 1 and 5.

FIG. 18 is a graph of the DNA-independent inhibition and DNA-dependent inhibition data (in terms of relative ATPase rate (s−1) versus concentration of inhibitor (μM)).

FIG. 19 provides a demonstration of bacterial growth inhibition for Peptide 1.

FIG. 20 provides a demonstration of killing activity of Peptide 1 against E. coli DH5α import mutant.

FIG. 21 shows graphically the relative potency, in terms of diameter of killing zone (mm) versus concentration of inhibitor (μM)).

FIG. 22 shows the haloassay for Peptide 5.

FIG. 23 shows photographs of seedlings, showing the action of Peptide 1 against A. thaliana ecotype Columbia (36 hours), for (a): seedlings, and (b) a close-up of the leaves.

FIG. 24 shows photographs of the germination of A. thaliana ecotype Columbia (36 hours) for wild type (left), 5 μM CFX (centre), and 100 μM Peptide 1 (right).

FIG. 25 shows photographs of the germination of A. thaliana ecotype Columbia (36 hours) for: no treatment (top left), 100 μM Peptide 2 (top right), 150 μM Peptide 2 (bottom left), and 200 μM Peptide 2 (bottom right).

FIG. 26 shows photographs of effects of Peptide 2 on 6-week old A. thaliana ecotype Columbia seedlings, for (a) no treatment (top left), 100 μM Peptide 2 (top right), 150 μM Peptide 2 (bottom left), and 200 μM Peptide 2 (bottom right), and (b) tumour-like growth observed at 150 μM Peptide 2.

FIG. 27 shows assay gels for thiazole compound and de-Boc-thiazole compound, and compares their inhibition of topoisomerase IIα-mediated relaxation of pBR322

FIG. 28 shows assay gels for microcin, oxazole compound, thiazole compound, Peptide 4, Peptide 5, and Peptide 1, and compares Inhibition of human topoisomerase I.

FIG. 29 shows assay gels for microcin, oxazole compound, thiazole compound, Peptide 1, Peptide 3, Peptide 4, and Peptide 5, and compares inhibition of human topoisomerase IIα.

FIG. 30 shows assay gels for oxazole compound, thiazole compound, microcin, Peptide 3, Peptide 1, Peptide 5, and Peptide 4, and compares inhibition of DNA relaxation by E. coli topoisomerase IV. (In vitro experiments: Decatenation catalysed by E. coli topoisomerase IV.)

FIG. 31 shows assay gels for microcin, oxazole compound, thiazole compound, Peptide 4, Peptide 5, and Peptide 3, and compares inhibition of E. coli topoisomerase IV decatenation.

FIG. 32 is a photograph showing the effects of 100 μM Peptide 1 on 4-week old Arabidopsis thaliana plants 24 hours after transfer to media containing the heterocyclic compound.

FIG. 33 shows photographs of the effects of Peptide 2 on 4-week old Arabidopsis thaliana plants. The plants were transferred to GM containing 200 μM Peptide 2 then observed for 5 days. Panel A: Undifferentiated, tumorous cell growth emerged from the meristematic regions (3× magnification); Panel B: tumorous cells emerging from the petiole (8× magnification); Panel C: tumorous cells emerging from the central meristem (8× magnification).

FIG. 34 shows photographs of the effects of heterocyclic compounds on 4-week old Arabidopsis thaliana plants. Compounds (0.5 μL) were spotted on to the expanded leaf or to the meristematic region (Panels G, H and I only). Panel A: plant before application of compound; Panel B: plant after application of 1 μL of Peptide 1; Panel C: onset of HR 5 minutes after application of Peptide 1; Panel D: plant after application of 1 μL of Peptide 1 on leaf and meristem; Panel E: systemic spread of HR 60 minutes after application, the red pigment is anthocyanin produced as a stress response; Panel F: HR spread through full leaf thickness after 60 minutes; Panel G: spread of HR 30 minutes after application of Peptide 1; Panel H: 24 hours after application to leaf; Panel I: 24 hours after application to leaf, necrosis has spread from the meristem out through the petioles and organellar replication zone.

FIG. 35 shows photographs of examples of the adherent cell cultures (8× magnification) used in these studies, two days after subculture into fresh CO₂ independent media. Left: HT-29, Right: HeLa.

FIG. 36 shows a photograph of the HT-29 cell culture plate after colourimetric MTT-based assay preparation. Treatments were duplicated and added to the microtitre plate as per the template to the above right. Purple (Column 1, and predominantly top right hand corner) represented viable cells, yellow (predominantly Columns 2, 3, and 4, and right hand end of Rows E, F, and G) represented dead cells.

Group 1: Columns 1-4; Group 2: Columns 5-8; Group 3; Columns 9-12;

Row A: Peptide 1, Peptide 3, DMSO;

Row B: 7.5, 14, 17, 22, 19, 37.5, 56, 75, 1%, 2%, 3%, 5%;

Row C: Peptide 5, Peptide 4, water;

Row D: 7.5, 19, 37.5, 75, 19, 37.5, 56, 75;

Row E: Thiazole, Oxazole, M-AMSA;

Row F: 7.5, 19, 37.5, 75, 19, 37.5, 56, 75, 25, 50, 75, 100;

Row G: deBoc Thiazole, Media, Camptothecin

Row H: 7.5, 19, 37.5, 75, 19, Microcin (x2), 75, 25, 50, 75, 100.

### DETAILED DESCRIPTION

Described herein are analogues of MccB17 component units I and II, as well as methods of making and using these compounds, which should extend the utility of MccB17 and provide a lead for further antibiotic drug developments. Also described herein are total synthesis methods for MccB17 component unit I and II analogues, for subsequent insertion into small peptidic structures.

One aspect of the present invention pertains to analogues of MccB17 component units I and II, as described herein. In a preferred embodiment, these analogues are hydrophilic.

These analogues may generally be described as amino acids or poly(amino acids) represented by the following formulae:

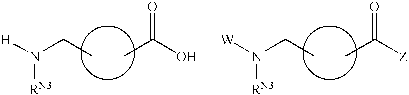

which incorporate a “mimic” amino acid residue represented by the following formula:

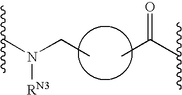

wherein W, Z, and R^{N3} are as defined below, and the circle represents a mono-heterocycle or a bis-heterocycle (i.e., two heterocycles linked together, but not fused together), wherein the heterocycle, or each of the two heterocycles, is a five membered ring having at least a first ring heteroatom that is N, and optionally a second ring heteroatom that is selected from N, O, and S (i.e., N₁, N₁O₁, N₁S₁, or N₂).

The phrase “having at least a first ring heteroatom that is N, and optionally a second ring heteroatom that is selected from N, O, and S” is intended to mean that no other ring heteroatoms are present, more specifically, that the ring has exactly 1 ring heterotom (that is N) or exactly 2 ring heteroatoms (one that is N, and a second that is selected from N, O, and S).

### Heterocycles

The heterocycle, or each of the two heterocycles, is independently selected from five membered rings having:
exactly one ring heteratom, wherein that ring heteroatom is N; or:
exactly two ring heteratoms, wherein those ring heteroatoms are N and O; or:
exactly two ring heteratoms, wherein those ring heteroatoms are N and S; or:
exactly two ring heteratoms, wherein those ring heteroatoms are N and N.

In one embodiment, the heterocycle, or each of the two heterocycles, is independently selected from five membered rings having:
exactly one ring heteratom, wherein that ring heteroatom is N.

In one embodiment, the heterocycle, or each of the two heterocycles, is independently selected from five membered rings having:
exactly two ring heteratoms, wherein those ring heteroatoms are N and O; or:
exactly two ring heteratoms, wherein those ring heteroatoms are N and S.

For example, in one embodiment, the heterocycle, or each of the two heterocycles, is selected from five membered rings derived from:
N₁: pyrrole (azole);
N₁O₁: oxazole (1,3-oxazole); isoxazole (1,2-oxazole);
N₁S₁: thiazole (1,3-thiazole); isothiazole (1,2-thiazole);
N₂: imidazole (1,3-diazole); pyrazole (1,2-diazole).

The phrase “derived from,” as used in this context, pertains to compounds which have the same ring atoms, and in the same orientation/configuration, as the parent heterocycle, and so include, for example, hydrogenated (e.g., partially saturated, fully saturated), carbonyl-substituted, and other substituted derivatives. For example, “pyrrolidone” and “N-methyl pyrrole” are both derived from “pyrrole”. Similarly, “N-methyl pyrrole” is, but “pyrrolidine” is not, an aromatic five membered ring derived from “pyrrole”.

Thus, such heterocycles include non-aromatic heterocycles, such as:

N₁: pyrrolidine (tetrahydropyrrole); pyrroline (e.g., 3-pyrroline, 2,5-dihydropyrrole);
2H-pyrrole or 3H-pyrrole (isopyrrole, isoazole);
N₂: imidazolidine; pyrazolidine (diazolidine); imidazoline; pyrazoline (dihydropyrazole);
N₁O₁: tetrahydrooxazole; dihydrooxazole; tetrahydroisoxazole;
dihydroisoxazole;
N₁S₁: thiazoline; thiazolidine;

Also, such heterocycles include carbonyl-substituted heterocycles, such as:

N₁: pyrrolidone (pyrrolidinone); 2-pyrrolidinone; 3-pyrrolidinone; 1,3-dihydro-pyrrol-2-one;
1,5-dihydro-pyrrol-2-one; 1,2-dihydro-pyrrol-3-one; pyrrol-2-one; pyrrol-3-one;
N₂: imidazolidone (imidazolidinone); pyrazolone (pyrazolinone);
N₁S₁: thiazolone, isothiazolone;
N₁O₁: oxazolinone.

In one preferred embodiment, the heterocycle, or each of the two heterocycles, is aromatic.

In one embodiment, the heterocycle, or each of the two heterocycles, is selected from five membered rings derived from: pyrrole, oxazole, isoxazole, thiazole, isothiazole, imidazole, and pyrazole.

In one embodiment, the heterocycle, or each of the two heterocycles, is selected from five membered rings derived from: pyrrole, oxazole, thiazole, and imidazole.

In one embodiment, the heterocycle, or each of the two heterocycles, is selected from five membered rings derived from: pyrrole, isoxazole, and isothiazole.

In one embodiment, the heterocycle, or each of the two heterocycles, is a five membered ring derived from pyrrole.

In one embodiment, the heterocycle, or each of the two heterocycles, is selected from five membered rings derived from: oxazole and thiazole.

In one embodiment, the heterocycle, or each of the two heterocycles, is a five membered ring derived from oxazole.

In one embodiment, the heterocycle, or each of the two heterocycles, is a five membered ring derived from thiazole.

In one embodiment, the heterocycle, or each of the two heterocycles, is selected from five membered rings derived from: isoxazole and isothiazole.

In one embodiment, the heterocycle, or each of the two heterocycles, is a five membered ring derived from isoxazole.

In one embodiment, the heterocycle, or each of the two heterocycles, is a five membered ring derived from isothiazole.

In one embodiment, the heterocycle, or each of the two heterocycles, is selected from five membered rings derived from: imidazole and pyrazole.

In one embodiment, the heterocycle, or each of the two heterocycles, is a five membered ring derived from imidazole.

In one embodiment, the heterocycle, or each of the two heterocycles, is a five membered ring derived from pyrazole.

In other embodiments, the phrase “is a five membered ring derived from” (or similar language) in the above embodiments is replaced with the word “is”, as in, for example: In one embodiment, the heterocycle, or each of the two heterocycles, is pyrrole.

If a heterocycle ring nitrogen atom is tridentate (e.g., —NH—, as in, for example, pyrrole), then it may be substituted (e.g., “N-substituted”), for example, with (1) C₁₋₆alkyl; (2) C₂₋₆alkenyl; (3) C₃₋₆cycloalkyl; (4) C₃₋₆cycloalkenyl; (5) C₆₋₁₄carboaryl; (6) C₅₋₁₄heteroaryl; (7) C₆₋₁₄carboaryl-C₁₋₆alkyl; or (8) C₅₋₁₄heteroaryl-C₁₋₆alkyl; each of which is itself optionally substituted (for example, with one or more of the substituents described next).

Additionally or alternatively, the heterocycle, or each of the heterocycles, may be substituted with one or more (e.g., 1, 2, 3) substituents, for example, selected from: (1) carboxylic acid; (2) ester; (3) amido or thioamido; (4) acyl; (5) halo; (6) cyano; (7) nitro; (8) hydroxy; (9) ether; (10) thiol; (11) thioether; (12) acyloxy; (13) carbamate; (14) amino; (15) acylamino or thioacylamino; (16) aminoacylamino or aminothioacylamino; (17) sulfonamino; (18) sulfonyl; (19) sulfonate; (20) sulfonamido; (21) C₁₋₆alkyl; (22) C₂₋₆alkenyl; (23) C₃₋₆cycloalkyl; (24) C₃₋₆cycloalkenyl; (25) C₆₋₁₄-carboaryl; (26) C₅₋₁₄heteroaryl; (27) C₆₋₁₄-carboaryl-C₁₋₆alkyl; and (28) C₅₋₁₄heteroaryl-C₁₋₆alkyl.

For example, in one embodiment, the heterocycle, or each of the two heterocycles, is pyrrole, and is optionally substituted, e.g., N-substituted, as described above.

All plausible combinations of the embodiments described above are explicitly disclosed herein as if each combination was individually recited.

For convenience, these analogues may be classified as “mono-heterocyclic” or “bis-heterocyclic” analogues.

### Mono-Heterocyclic Analogues

In one embodiment, the compounds are selected from compounds of the following general formulae:

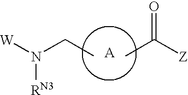

wherein:
W is independently —H or a peptide group;
Z is independently —OH or a peptide group;
wherein each peptide group, if present, is:
   an amino acid group and comprises exactly one amino acid, or:
   a poly(amino acid) group and comprises two or more amino acids;
R^{N3} is independently: —H, C₁₋₆alkyl, C₂₋₆alkenyl, C₃₋₆cycloalkyl, or C₃₋₆cycloalkenyl, C₆₋₁₄carboaryl, C₅₋₁₄heteroaryl, C₆₋₁₄carboaryl-C₁₋₆alkyl, or C₅₋₁₄heteroaryl-C₁₋₆alkyl, and is optionally substituted;
the circle “A” denotes a mono-heterocycle five membered ring (A-ring) having at least a first ring heteroatom that is N, and optionally a second ring heteroatom that is selected from N, O, and S;
the group —C(═O)-Z is attached to a first ring atom of said five membered ring;
the group —CH₂—NR^{N3}—W is attached to a second ring atom of said five membered ring;
the A-ring is optionally additionally independently substituted (for example, with one or more substituents as described above for possible heterocycle substituents);
and pharmaceutically acceptable salts, amides, esters, solvates, and hydrates thereof.

As mentioned above, the group —CH₂—NR^{N3}—W is attached to a first ring atom of said five membered ring (A-ring). That first ring atom corresponds to a hydrogen-bearing ring atom (i.e., carbon ring atom, nitrogen ring atom) of the parent heterocycle, for example, a hydrogen bearing carbon ring atom of pyrrole, or the hydrogen-bearing nitrogen ring atom of pyrrole.

Similarly, the group —C(═O)-Z is attached to a second ring atom of said five membered ring (A-ring). That second ring atom corresponds to a hydrogen-bearing ring atom (i.e., carbon ring atom, nitrogen ring atom) of the parent heterocycle, for example, a hydrogen bearing carbon ring atom of pyrrole, or the hydrogen bearing nitrogen ring atom of pyrrole.

In one embodiment, the optional second ring heteroatom is not present.

In one embodiment, the second ring heteroatom, if present, is selected from O and S.

In one embodiment, the mono-heterocyclic group is derived from: pyrrole, imidazole, oxazole, thiazole, pyrazole, isoxazole, or isothiazole. (Again, note that, in general, “derived from” includes hydrogenated (e.g., partially saturated, fully saturated), carbonyl-substituted, and other substituted derivatives.)

In one embodiment, additionally, the A-ring is aromatic, that is, the circle “A” denotes a five membered aromatic ring (A-ring).

In one embodiment, circle A denotes a five membered ring derived from: pyrrole, imidazole, oxazole, thiazole, pyrazole, isoxazole, or isothiazole.

In one embodiment, circle A denotes a five membered ring derived from: pyrrole, imidazole, oxazole, or thiazole.

In one embodiment, circle A denotes a five membered ring derived from: pyrrole.

In one embodiment, circle A denotes a five membered ring derived from: oxazole or thiazole.

In one embodiment, circle A denotes is a five membered ring derived from oxazole.

In one embodiment, circle A denotes a five membered ring derived from thiazole.

In one embodiment, circle A denotes a five membered ring derived from: isoxazole or isothiazole.

In one embodiment, circle A denotes a five membered ring derived from isoxazole.

In one embodiment, circle A denotes a five membered ring derived from isothiazole.

In one embodiment, circle A denotes a five membered ring derived from: imidazole or pyrazole.

In one embodiment, circle A denotes a five membered ring derived from imidazole.

In one embodiment, circle A denotes a five membered ring derived from pyrazole.

In other embodiments, the phrase “denotes a five membered ring derived from” (or similar language) in the above embodiments is replaced with the word “denotes”, as in, for example: In one embodiment, circle A denotes pyrrole.

In one embodiment, the compounds are selected from compounds of the following general formulae:

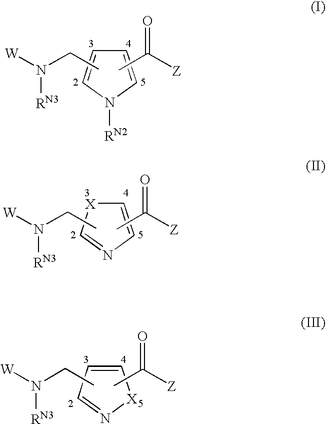

wherein:
W is independently —H or a peptide group;
Z is independently —OH or a peptide group;
wherein each peptide group, if present, is:
   an amino acid group and comprises exactly one amino acid, or:
   a poly(amino acid) group and comprises two or more amino acids;
X is independently —NR^{N1}—, —O—, or —S—;
each of R^{N1}, R^{N2}, and R^{N3}, if present, is independently: —H, C₁₋₆alkyl, C₂₋₆alkenyl, C₃₋₆cycloalkyl, or C₃₋₆cycloalkenyl, C₆₋₁₄carboaryl, C₅₋₁₄heteroaryl, C₆₋₁₄carboaryl-C₁₋₆alkyl, C₅₋₁₄heteroaryl-C₁₋₆alkyl, and is optionally substituted;
the group —CH₂—N(R^{N3})—W is independently attached at the 2-, 3-, 4-, or 5-ring position;
the group —C(═O)-Z is independently attached at one of the remaining ring positions;
the five membered heterocyclic ring is optionally additionally independently substituted (for example, with one or more substituents as described above for possible heterocycle substituents);
and pharmaceutically acceptable salts, amides, esters, solvates, and hydrates thereof.

Compounds of Formula (I) are “pyrroles”.

Compounds of Formula (II) are “imidazoles” (where X is —NR^{N1}—), “oxazoles” (where X is —O—) and “thiazoles” (where X is —S—).

Compounds of Formula (III) are “pyrazoles” (where X is —NR^{N1}—), “isoxazoles” (where X is —O—) and “isothiazoles” (where X is —S—).

In one preferred embodiment, the compounds are of Formula (I).

In one preferred embodiment, the compounds are of Formula (I), and:

the group —CH₂—N(R^{N3})—W is independently attached at the 2- or 3-ring position; and
the group —C(═O)-Z is independently attached at the 4- or 5-ring position.

In one preferred embodiment, the compounds are of Formula (I), and:

the group —CH₂—N(R^{N3})—W is independently attached at the 2- or 3-ring position; and
the group —C(═O)-Z is independently attached at the 5-ring position;
for example, as in the following formulae:

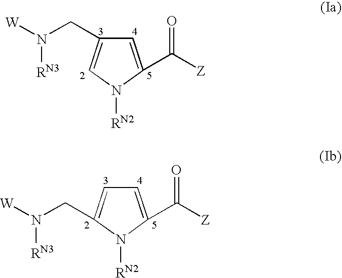

In one preferred embodiment, the compounds are of Formula (I), and:

the group —CH₂—N(R^{N3})—W is independently attached at the 2-ring position; and
the group —C(═O)-Z is independently attached at the 5-ring position;
for example, as in the formula (Ib).

In one preferred embodiment, the compounds are of Formula (I), and:

the group —CH₂—N(R^{N3})—W is independently attached at the 3-ring position; and
the group —C(═O)-Z is independently attached at the 5-ring position;
for example, as in the formula (Ia).

In one embodiment, the compounds are of Formula (II).

In one embodiment, the compounds are of Formula (II), and:

the group —CH₂—N(R^{N3})—W is independently attached at the 2-ring position; and
the group —C(═O)-Z is independently attached at the 4- or 5-ring position;
for example, as in the following formulae:

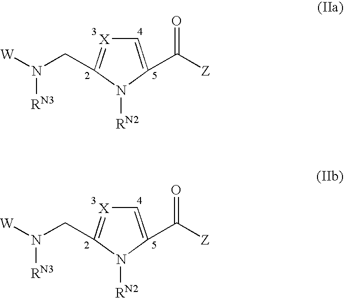

In one embodiment, the compounds are of Formula (II), and:

the group —CH₂—N(R^{N3})—W is independently attached at the 2-ring position; and
the group —C(═O)-Z is independently attached at the 4-ring position;
for example, as in Formula (IIb).

In one embodiment, the compounds are of Formula (II), and:

the group —CH₂—N(R^{N3})—W is independently attached at the 2-ring position; and
the group —C(═O)-Z is independently attached at the 5-ring position;
for example, as in Formula (IIa).

In one embodiment, the compounds are of Formula (III).

In one embodiment, the compounds are of Formula (III), and:

the group —CH₂—N(R^{N3})—W is independently attached at the 2- or 3-ring position; and
the group —C(═O)-Z is independently attached at the 4-ring position.

In one embodiment, the compounds are of Formula (III), and:

the group —CH₂—N(R^{N3})—W is independently attached at the 4-ring position; and
the group —C(═O)-Z is independently attached at the 2-ring position; or
the group —CH₂—N(R^{N3})—W is independently attached at the 2-ring position; and
the group —C(═O)-Z is independently attached at the 4-ring position;
for example, as in the following formulae:

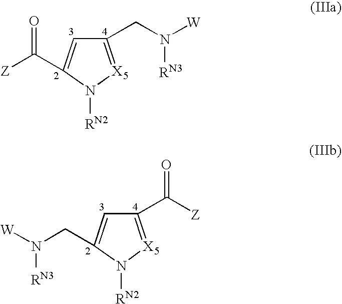

In one preferred embodiment, the compounds are of Formula (III), and:

the group —CH₂—N(R^{N3})—W is independently attached at the 4-ring position; and
the group —C(═O)-Z is independently attached at the 2-ring position;
for example, as in the formula (IIIa).

In one preferred embodiment, the compounds are of Formula (III), and:

the group —CH₂—N(R^{N3})—W is independently attached at the 2-ring position; and
the group —C(═O)-Z is independently attached at the 4-ring position;
for example, as in the formula (IIIb).

In one preferred embodiment, R^{N1}, if present, is independently —H or C₁₋₆alkyl.

In one preferred embodiment, R^{N1}, if present, is independently —H or -Me.

In one preferred embodiment, R^{N1}, if present, is independently —H.

In one preferred embodiment, R^{N2}, if present, is independently —H or C₁₋₆alkyl.

In one preferred embodiment, R^{N2}, if present, is independently —H or -Me.

In one preferred embodiment, R^{N2}, if present, is independently —H.

In one embodiment, X, if present, is independently —O— or —S—.

In one embodiment, X, if present, is independently —O— (“oxazoles” and “isoxazole”).

In one embodiment, X, if present, is independently —S— (“thiazoles” and “isothiazoles”).

All plausible combinations of the embodiments described above are explicitly disclosed herein as if each combination was individually recited.

### Bis-Heterocyclic Analogues

In one embodiment, the compounds are selected from compounds of the following general formula:

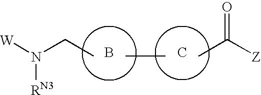

wherein:
W is independently —H or a peptide group;
Z is independently —OH or a peptide group;
wherein each peptide group, if present, is:
   an amino acid group and comprises exactly one amino acid, or:
   a poly(amino acid) group and comprises two or more amino acids;
R^{N3} is independently: —H, C₁₋₆alkyl, C₂₋₆alkenyl, C₃₋₆cycloalkyl, or C₃₋₆cycloalkenyl, C₆₋₁₄carboaryl, C₅₋₁₄heteroaryl, C₆₋₁₄carboaryl-C₁₋₆alkyl, C₅₋₁₄heteroaryl-C₁₋₆alkyl, and is optionally substituted;
the circle “B” denotes a first mono-heterocycle five membered ring (B-ring) having at least a first ring heteroatom that is N, and optionally a second ring heteroatom that is selected from N, O, and S;
the circle “C” denotes a second mono-heterocycle five membered ring (C-ring) having at least a first ring heteroatom that is N, and optionally a second ring heteroatom that is selected from N, O, and S;
wherein a first ring atom of said first five membered ring (B-ring) is linked by a covalent bond to a first ring atom of said second five membered ring (C-ring);
the group —CH₂—NR^{N3}—W is attached to a second ring atom of said first five membered ring (B-ring);
the group —C(═O)-Z is attached to a second ring atom of said second five membered ring (C-ring);
each of the B-ring and C-ring is optionally additionally independently substituted (for example, with one or more substituents as described above for possible heterocycle substituents);
and pharmaceutically acceptable salts, amides, esters, solvates, and hydrates thereof.

Only one ring atom of said first five membered ring (B-ring) is linked to a ring atom of said second five membered ring (C-ring); that is, the first and second five membered rings are not fused.

As mentioned above, the group —CH₂—NR^{N3}—W is attached to a second ring atom of said first five membered ring (B-ring). That second ring atom corresponds to a hydrogen-bearing ring atom (i.e., carbon ring atom, nitrogen ring atom) of the parent heterocycle, for example, a hydrogen bearing carbon ring atom of pyrrole, or the hydrogen bearing nitrogen ring atom of pyrrole.

Similarly, the group —C(═O)-Z is attached to a second ring atom of said second five membered ring (C-ring). That second ring atom corresponds to a hydrogen-bearing ring atom (i.e., carbon ring atom, nitrogen ring atom) of the parent heterocycle, for example, a hydrogen bearing carbon ring atom of pyrrole, or the hydrogen bearing nitrogen ring atom of pyrrole.

In one embodiment, additionally, the B-ring is aromatic, that is, the circle “B” denotes a first five membered aromatic ring (B-ring).

In one embodiment, additionally, the C-ring is aromatic, that is, the circle “C” denotes a second five membered aromatic ring (C-ring).

In one embodiment, additionally, both the B-ring is aromatic and the C-ring is aromatic, that is, the circle “B” denotes a first five membered aromatic ring (B-ring), and the circle “C” denotes a second five membered aromatic ring (C-ring).

In one embodiment, each of circle B and circle C independently denotes a five membered ring derived from: pyrrole, imidazole, oxazole, thiazole, pyrazole, isoxazole, or isothiazole. (Again, note that, in general, “derived from” includes hydrogenated (e.g., partially saturated, fully saturated), carbonyl-substituted, and other substituted derivatives.)

In one embodiment, each of circle B and circle C independently denotes a five membered ring derived from: pyrrole, imidazole, oxazole, or thiazole.

In one embodiment, each of circle B and circle C independently denotes a five membered ring derived from: pyrrole, isoxazole, or isothiazole.

In one embodiment, each of circle B and circle C independently denotes a five membered ring derived from: pyrrole.

In one embodiment, at least one of circle B and circle C denotes a five membered ring derived from: pyrrole.

In one embodiment, one of circle B and circle C denotes a five membered ring derived from: pyrrole; and the other denotes a five membered ring derived from: pyrrole, oxazole, or thiazole.

In one embodiment, one of circle B and circle C independently denotes a five membered ring derived from: oxazole; and the other denotes a five membered ring derived from: thiazole.

In one embodiment, circle B and circle C denote identical five membered rings (e.g., both circle B and circle C denote pyrrole).

In one embodiment, circle B and circle C denote different five membered rings (e.g., one derived from pyrrole, one derived from thiazole).

In other embodiments, the phrase “denotes a five membered ring derived from” (or similar language) in the above embodiments is replaced with the word “denotes”, as in,

for example: In one embodiment, each of circle B and circle C independently denotes pyrrole.

In one embodiment, the first ring heteroatom (N) of said first five membered ring (B-ring) is linked by a covalent bond to a carbon ring atom of said second five membered ring (C-ring), for example, as in:

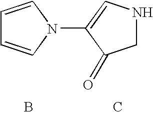

In the above example, the group —CH₂—NR^{N3}—W is attached to one of the hydrogen-bearing carbon ring-atoms of the B-ring, and the group —C(═O)-Z is attached to one of the hydrogen-bearing carbon ring atoms of the C-ring.

In one embodiment, a carbon ring atom of said first five membered ring (B-ring) is linked by a covalent bond to a carbon atom of said second five membered ring (C-ring), for example, as in:

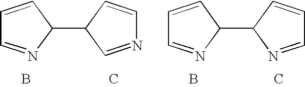

In one embodiment, a carbon ring atom of said first five membered ring (B-ring) that is adjacent to its first ring heteroatom (N) is linked by a covalent bond to a carbon atom of said second five membered ring (C-ring) that is adjacent to its first ring heteroatom, for example, as in:

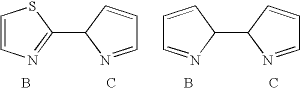

In one embodiment, each of circle B and circle C independently denotes a five membered ring derived from: pyrrole, oxazole, or thiazole; and a carbon ring atom of said first five membered ring (B-ring) that is adjacent to its first ring heteroatom (N) is linked by a covalent bond to a carbon atom of said second five membered ring (C-ring) that is adjacent to its first ring heteroatom, for example, as in:

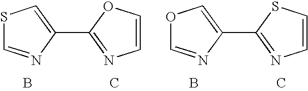

In one embodiment, the bis-heterocyclic group (i.e., B-C) is derived from: pyrrolyl-pyrrole; pyrrolyl-oxazole; pyrrolyl-thiazole; pyrrolyl-pyrazole; oxazolyl-pyrrole; oxazolyl-oxazole; oxazolyl-thiazole; oxazolyl-pyrazole; thiazolyl-pyrrole; thiazolyl-oxazole; thiazolyl-thiazole; thiazolyl-pyrazole; pyrazolyl-pyrrole; pyrazolyl-oxazole; pyrazolyl-thiazole; or pyrazolyl-pyrazole. (Again, note that, in general, “derived from” includes hydrogenated (e.g., partially saturated, fully saturated), carbonyl-substituted, and other substituted derivatives.)

In one embodiment, the bis-heterocyclic group (i.e., B-C) is derived from: oxazolyl-thiazole; or thiazolyl-oxazole.

In other embodiments, the phrase “is derived from” in the above embodiment is replaced with the word “is”, as in, for example: In one embodiment, the bis-heterocyclic group is: pyrrolyl-pyrrole, etc.

In one embodiment, each of the two heterocycles of the bis-heterocyclic group (i.e., the B-ring and the C-ring), is aromatic, e.g., as in an “aromatic bis-heterocyclic group”.

In one embodiment, the compounds are selected from compounds of the following general formula:

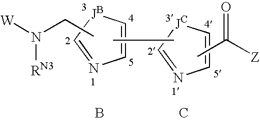

wherein:
W is independently —H or a peptide group;
Z is independently —OH or a peptide group;
wherein each peptide group, if present, is:
   an amino acid group and comprises exactly one amino acid, or:
   a poly(amino acid) group and comprises two or more amino acids;
R^{N3} is independently: —H, C₁₋₆alkyl, C₂₋₆alkenyl, C₃₋₆cycloalkyl, or C₃₋₆cycloalkenyl, C₆₋₁₄carboaryl, C₅₋₁₄heteroaryl, C₆₋₁₄carboaryl-C₁₋₆alkyl, C₆₋₁₄heteroaryl-C₁₋₆alkyl, and is optionally substituted;
each of J^{B} and J^{C} is independently —O— or —S—;
the group —CH₂—N(R^{N3})—W is independently attached at the 2-, 4-, or 5-ring position;
the group —C(═O)-Z is independently attached at the 2′, 4′, or 5′-ring position;
the B-ring and C-ring are linked by a covalent bond between one of the remaining 2-, 4-, or 5-ring positions and one of the remaining 2′, 4′, or 5′-ring positions;
each of the B-ring and C-ring is optionally additionally independently substituted (for example, with one or more substituents as described above for possible heterocycle substituents);
   and pharmaceutically acceptable salts, amides, esters, solvates, and hydrates thereof.

In one embodiment, one of J^{B} and J^{C} is —O— and the other is —S—.

In one embodiment, J^{B} is —O— and J^{C} is —S—.

In one embodiment, J^{B} is —S— and J^{C} is —O—.

In one embodiment, J^{B} is —O— and J^{C} is —O—.

In one embodiment, J^{B} is —S— and J^{C} is —S—.

In one embodiment:
the group —CH₂—N(R^{N3})—W is independently attached at the 2-ring position;
the group —C(═O)-Z is independently attached at the 4′ or 5′-ring position;
the B-ring and C-ring are linked by a covalent bond between the 4- or 5-ring position and the 2′-ring position.

In one embodiment:
the group —CH₂—N(R^{N3})—W is independently attached at the 2-ring position;
the group —C(═O)-Z is independently attached at the 5′-ring position;
the B-ring and C-ring are linked by a covalent bond between the 5-ring position and the 2′-ring position,
as in, for example:

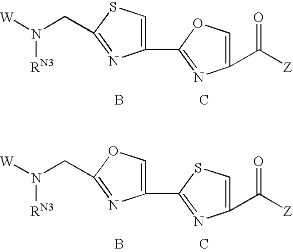

In one embodiment, the compounds are selected from compounds of the following general formula:

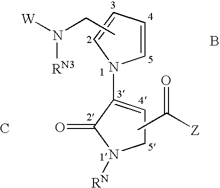

wherein:
W is independently —H or a peptide group;
Z is independently —OH or a peptide group;
wherein each peptide group, if present, is:
   an amino acid group and comprises exactly one amino acid, or:
   a poly(amino acid) group and comprises two or more amino acids;
each of R^{N3} and R^{N} is independently: —H, C₁₋₆alkyl, C₂₋₆alkenyl, C₃₋₆cycloalkyl, or C₃₋₆cycloalkenyl, C₆₋₁₄carboaryl, C₅₋₁₄heteroaryl, C₆₋₁₄carboaryl-C₁₋₆alkyl, C₅₋₁₄heteroaryl-C₁₋₆alkyl, and is optionally substituted;
the group —CH₂—N(R^{N3})—W is independently attached at the 2-, 3-, 4-, or 5-ring position;
the group —C(═O)-Z is independently attached at the 4′ or 5′-ring position;
each of the B-ring and C-ring is optionally additionally independently substituted (for example, with one or more substituents as described above for possible heterocycle substituents);
and pharmaceutically acceptable salts, amides, esters, solvates, and hydrates thereof.

In one embodiment:
the group —CH₂—N(R^{N3})—W is independently attached at the 2- or 3-ring position;
the group —C(═O)-Z is independently attached at the 4′ or 5′-ring position.

In one embodiment:
the group —CH₂—N(R^{N3})—W is independently attached at the 2- or 3-ring position;
the group —C(═O)-Z is independently attached at the 4′-ring position.

All plausible combinations of the embodiments described above are explicitly disclosed herein as if each combination was individually recited.

### The Group R^{N3}

The group R^{N3} is independently: —H, C₁₋₆alkyl, C₂₋₆alkenyl, C₃₋₆cycloalkyl, or C₃₋₆cycloalkenyl, C₆₋₁₄carboaryl, C₅₋₁₄heteroaryl, C₆₋₁₄carboaryl-C₁₋₆alkyl, C₅₋₁₄heteroaryl-C₁₋₆alkyl, and is optionally substituted.

Examples of optional substituents include those discussed above as possible heterocycle substituents.

In one preferred embodiment, R^{N3} is independently —H, C₁₋₆alkyl, or C₆₋₁₄carboaryl-C₁₋₆alkyl.

In one preferred embodiment, R^{N3} is independently —H or C₁₋₆alkyl.

In one preferred embodiment, R^{N3} is independently —H or -Me.

In one preferred embodiment, R^{N3} is independently —H.

### The Groups W and Z

The Group W is independently —H or a peptide group.

The Group Z is independently —OH or a peptide group.

In one embodiment, at least one of W and Z is a peptide group.

In one embodiment, each of W and Z is a peptide group.

In one embodiment:
W is independently —H; and
Z is independently —OH or a peptide group.

In one embodiment:
W is independently —H; and
Z is independently —OH.

In one embodiment:
W is independently —H; and
Z is independently a peptide group.

In one embodiment:
W is independently a peptide group; and
Z is independently —OH or a peptide group.

In one embodiment:
W is independently a peptide group; and
Z is independently —OH.

In one embodiment:
W is independently a peptide group; and
Z is independently a peptide group.

In one embodiment:
W is independently —H or a peptide group; and
Z is independently —OH or a peptide group.

In one embodiment:
W is independently a —H or a peptide group; and
Z is independently a peptide group.

In one embodiment:
W is independently a —H or a peptide group; and
Z is independently —OH.

The term “peptide group,” as used in this context, pertains to both amino acid groups (i.e., groups comprising a single amino acid) and poly(amino acid) groups (i.e., groups comprising two or more amino acids) (e.g., polypeptide groups, oligopeptide groups), linked via an amide bond.

The phrase “linked via an amide bond” means that, when Z is a peptide group, the —C(═O)— group of the —C(═O)Z group forms part of an amide bond; and that, when W is a peptide group, the —N(R^{N3})— group of the —CH₂—N(R^{N3})—W group forms part of an amide bond. This is illustrated in the following examples:

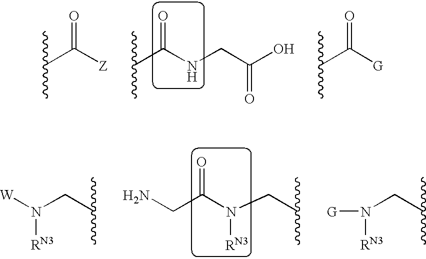

In one embodiment, when two peptide groups are present (i.e., when each of W and Z is a peptide group), the two peptide groups are identical.

In one embodiment, when two peptide groups are present (i.e., when each of W and Z is a peptide group), the two peptide groups are different.

In one embodiment, the peptide group, if only one is present, or one of (e.g., exactly one of, at least one of) the peptide groups, if two are present, is an amino acid group, that is, comprises exactly one amino acid.

In one embodiment, the peptide group, if only one is present, or one of (e.g., exactly one of, at least one of) the peptide groups, if two are present, is a poly(amino acid) group, that is, comprises two or more amino acids.

In one embodiment, when two peptide groups are present (i.e., when each of W and Z is a peptide group), each peptide group is independently an amino acid group.

In one embodiment, when two peptide groups are present (i.e., when each of W and Z is a peptide group), each peptide group is independently a poly(amino acid) group.

In one embodiment, when two peptide groups are present (i.e., when each of W and Z is a peptide group), one peptide group is independently an amino acid group, and the other peptide group is independently a poly(amino acid) group.

In one embodiment, when two peptide groups are present (i.e., when each of W and Z is a peptide group), W is independently an amino acid group, and Z is independently a poly(amino acid) group.

In one embodiment, when two peptide groups are present (i.e., when each of W and Z is a peptide group), Z is independently an amino acid group, and W is independently a poly(amino acid) group.

In one embodiment, the or each poly(amino acid) groups is selected from poly(amino acid) groups having from 2 to 10 amino acids, for example, from 2 to 5 amino acids, for example, 2, 3, 4, or 5 amino acids.

In one embodiment, the amino acid of said amino acid group, if present, or each amino acid of said poly(amino acid) group, if present, is a non-sterically hindered amino acid.

In one embodiment, the amino acid of said amino acid group, if present, or each amino acid of said poly(amino acid) group, if present, is a naturally occurring α-amino acid.

In one embodiment, the amino acid of said amino acid group, if present, or each amino acid of said poly(amino acid) group, if present, is a naturally occurring non-sterically hindered α-amino acid.

In one embodiment, the amino acid of said amino acid group, if present, or each amino acid of said poly(amino acid) group, if present, is selected from glycine (Gly, G), alanine (Ala, A), and glutamine (Gln, Q).

In one embodiment, the or each amino acid independently is, or additionally is, an α-amino acid which, if chiral, is in the L configuration (i.e., each chiral amino acid α-carbon is in the S configuration). For example, in one embodiment, the or each amino acid is selected from glycine (glycine is not chiral), L-alanine, and L-glutamine.

In one embodiment, the group W—NR^{N3}—CH₂— is H-[AA¹]ₙ-NR^{N3}—CH₂—, wherein AA¹ is an amino acid group (e.g., as defined above) and n is an integer from 1 to 10, for example, from 1 to 5, for example, 1, 2, 3, 4, or 5. In one embodiment (where W is a poly(amino acid) group), n is an integer from 2 to 10, for example, from 2 to 5, for example, 2, 3, 4, or 5.

In one embodiment, the group AA¹ is a group of the formula —NH—R—C(═O)— wherein R is an organic group (i.e., a group having, at least, carbon and hydrogen atoms) having from 1 to 10 atoms selected from C, N, O, and S, for example, a group of the formula —CHR^{AA}—, wherein R^{AA} is an α-amino acid side-chain.

In one embodiment, the group W—NR^{N3}—CH₂— is H—[NH—CHR^{AA}—C(═O)]ₙ—NR^{N3}—CH₂—, wherein R^{AA} is an α-amino acid side-chain and n is an integer from 1 to 10, for example, from 1 to 5, for example, 1, 2, 3, 4, or 5. In one embodiment (where W is a poly(amino acid) group), n is an integer from 2 to 10, for example, from 2 to 5, for example, 2, 3, 4, or 5.

In one embodiment, the or each α-amino acid side-chain is independently selected from the α-amino acid side-chains of naturally occurring α-amino acids.

In one embodiment, the or each α-amino acid side-chain is independently selected from the α-amino acid side-chains of naturally occurring non-sterically hindered α-amino acids.

In one embodiment, the or each α-amino acid side-chain is independently selected from the α-amino acid side-chains of glycine (Gly, G), alanine (Ala, A), and glutamine (Gln, Q).

For example, in one embodiment, W is a glycine group and the group —CH₂—NR^{N3}—W is:

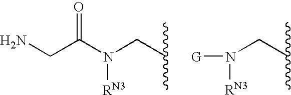

For example, in one embodiment, W is -AGQ, and the group —CH₂—NR^{N3}—W is (wherein, preferably, each chiral amino acid α-carbon is in the S configuration):

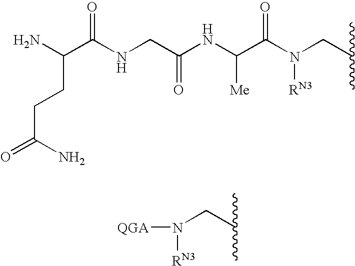

In one embodiment, the group —C(═O)-Z is —C(═O)—[AA²]ₘ—OH, wherein AA² is an amino acid group (e.g., as defined above) and m is an integer from 1 to 10, for example, from 1 to 5, for example, 1, 2, 3, 4, or 5. In one embodiment (where Z is a poly(amino acid) group), m is an integer from 2 to 10, for example, from 2 to 5, for example, 2, 3, 4, or 5.

In one embodiment, the group AA² is a group of the formula —NH—R—C(═O)— wherein R is an organic group having from 1 to 10 atoms selected from C, N, O, and S, for example, a group of the formula —CHR^{AA}—, wherein R^{AA} is an α-amino acid side-chain.

In one embodiment, the group —C(═O)-Z is —C(═O)—[NH—CHR^{AA}—C(═O)]ₘ—OH, wherein R^{AA} is an α-amino acid side-chain (e.g., as defined above) and m is an integer from 1 to 10, for example, from 1 to 5, for example, 1, 2, 3, 4, or 5. In one embodiment (where Z is a poly(amino acid) group), m is an integer from 2 to 10, for example, from 2 to 5, for example, 2, 3, 4, or 5.

For example, in one embodiment, Z is a glycine group and the group —C(═O)-Z is:

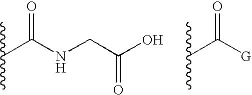

For example, in one embodiment, Z is -GGA, and the group —C(═O)-Z is (wherein, preferably, each chiral amino acid α-carbon is in the S configuration):

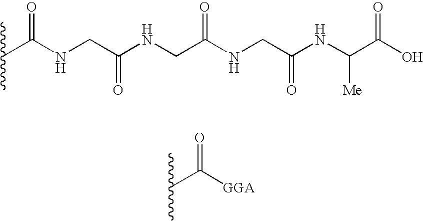

In one embodiment, the terminal —NH₂ and —COOH groups of W and Z (and optionally other —NH₂ and —COOH groups of W and Z, if present) may independently be derivatized, protected, etc. For example, a —NH₂ group may be derivatized to form a substituted amine (e.g., substituted with one or two groups as defined for R^{N3}, e.g., —NR₂, where each R is independently as defined for R^{N3}), an amide (e.g., —NHCOR, where R is as defined for R^{N3}, but is not —H), etc. For example, a —COOH group may be derivatized to form an ester (e.g., —COOR, where R is as defined for R^{N3}, but is not —H), an amide (e.g., —CONR₂, where each R is as defined for R^{N3}), etc.

In one embodiment, the nitrogen atom of any amide bonds (>N—C(═O)—) in W and Z, if present, independently bears a group as defined for R^{N3}, for example, is independently unsubstituted (i.e., as —NH—C(═O)—) or substituted (i.e., as —NR—C(═O)—), e.g., with C₁₋₆alkyl, e.g., -Me.

All plausible combinations of the embodiments described above are explicitly disclosed herein as if each combination was individually recited.

### Some Preferred Examples

Examples of some preferred compounds (having a mono-heterocycle) include the following (wherein, preferably, each chiral amino acid α-carbon is in the S configuration):

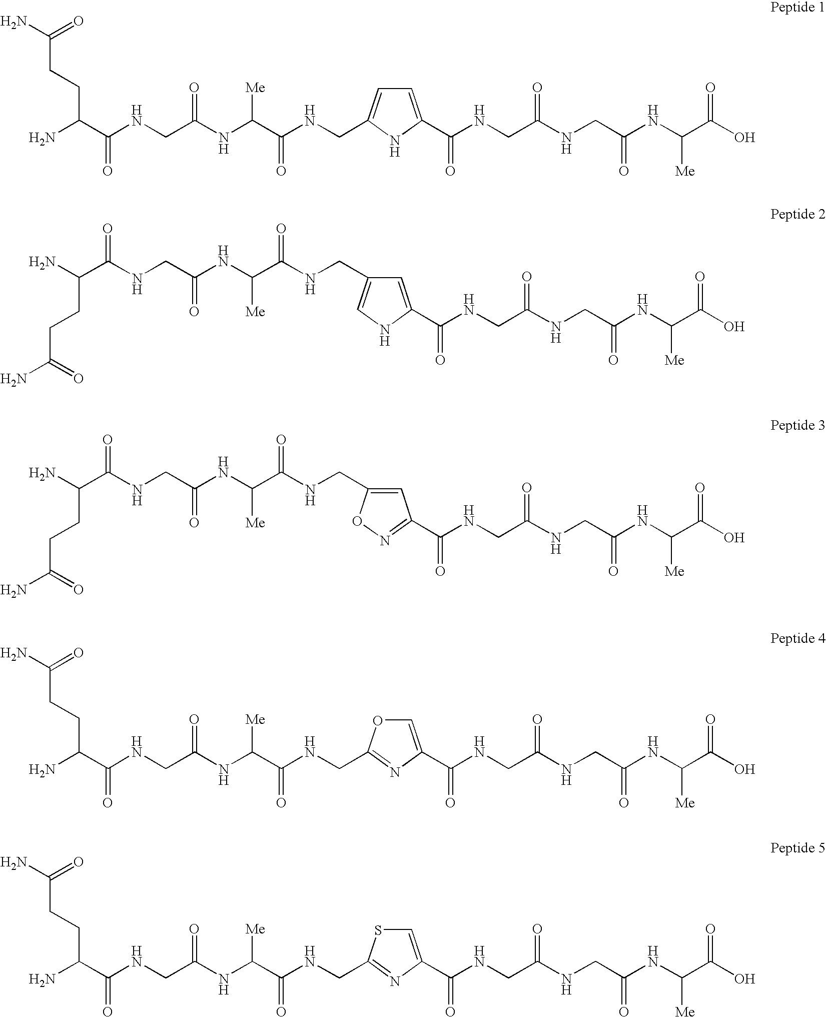

In one preferred embodiment, the compound is selected from peptides 1 through 5, and pharmaceutically acceptable salts, amides, esters, solvates, and hydrates thereof.

In one preferred embodiment, the compound is selected from Peptide 1, Peptide 2, Peptide 4, and Peptide 5, and pharmaceutically acceptable salts, amides, esters, solvates, and hydrates thereof.

In one preferred embodiment, the compound is selected from Peptide 1 and Peptide 2, and pharmaceutically acceptable salts, amides, esters, solvates, and hydrates thereof.

Some examples of preferred compounds (having a bis-heterocycle) include the following (wherein, preferably, each chiral amino acid α-carbon is in the S configuration):

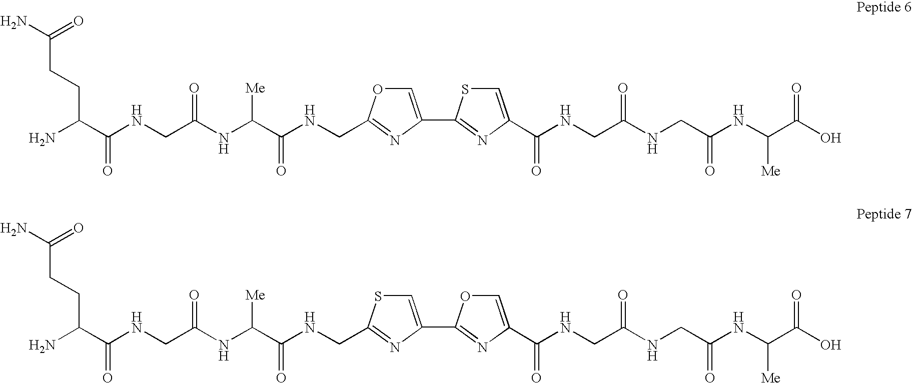

In one embodiment, the compound is selected from the compounds shown in the Examples below, and pharmaceutically acceptable salts, amides, esters, solvates, and hydrates thereof.

### Compositions

One aspect of the present invention pertains to a composition comprising a compound of the present invention, as described herein, and a carrier or diluent.

One aspect of the present invention pertains to a composition comprising a compound of the present invention, as described herein, and a pharmaceutically acceptable carrier or diluent.

### Chemical Synthesis

Several methods for the chemical synthesis of compounds of the present invention are described herein. These and/or other well known methods may be modified and/or adapted in known ways in order to facilitate the synthesis of additional compounds within the scope of the present invention.

### Uses

The compounds described herein are useful, for example, in the treatment of diseases and conditions that are ameliorated by the inhibition of DNA Gyrase, such as, for example, bacterial infections, cancer, etc.

### Use in Methods of Inhibiting DNA Gyrase

One aspect of the present invention pertains to a method of inhibiting DNA Gyrase activity in a cell, in vitro or in vivo, comprising contacting the cell with an effective amount of a compound, as described herein.

Suitable assays for determining DNA Gyrase inhibition are described in the Examples below.

### Use in Methods of Therapy

Another aspect of the present invention pertains to a compound as described herein for use in a method of treatment of the human or animal body by therapy.

### Use in the Manufacture of Medicaments

Another aspect of the present invention pertains to use of a compound, as described herein, in the manufacture of a medicament for use in treatment.

### Methods of Treatment

Another aspect of the present invention pertains to a method of treatment comprising administering to a patient in need of treatment a therapeutically effective amount of a compound as described herein, preferably in the form of a pharmaceutical composition.

### Conditions

In one embodiment (e.g., of use in methods of therapy, of use in the manufacture of medicaments, of methods of treatment), the treatment is treatment of a disease or condition that is ameliorated by the inhibition of DNA Gyrase.

### Bacterial Infections

In one embodiment (e.g., of use in methods of therapy, of use in the manufacture of medicaments, of methods of treatment), the treatment is treatment of a bacterial infection, e.g., in a patient.

In one embodiment, the bacterial infection is selected from infections with one or more of the following: Streptococcus pneumoniae, Streptococcus pyogenes, Enterococcus fecalis, Enterococcus faecium, Klebsiella pneumoniae, Enterobacter sps., Proteus sps., Pseudomonas aeruginosa, E. coli, Serratia marcesens, S. aureus, Coag. Neg. Staph., Acinetobacter sps., Salmonella sps, Shigella sps., Helicobacter pylori, Mycobacterium tuberculosis, Mycobacterium avium, Mycobacterium intracellulare, Mycobacterium fortuitum, Mycobacterium chelonae, Mycobacterium kansasii, Haemophilus influenzae, Stenotrophomonas maltophilia, Streptococcus agalactiae, and Methicillin Resistant Staphylococcus Aureus (MRSA).

The compositions and methods will therefore be useful for controlling, treating or reducing the advancement, severity or effects of nosocomial infections (also known as community acquired infections, e.g., a new disorder, not the patient's original condition, that is acquired in a healthcare setting, for example, in a hospital, or as a result of medical care, for example, a hospital-acquired infection) or non-nosocomial infections. Examples of nosocomial uses include the treatment of: urinary tract infections, pneumonia, surgical wound infections, bone and joint infections, and bloodstream infections. Examples of non-nosocomial uses include the treatment of urinary tract infections, pneumonia, prostatitis, skin and soft tissue infections, bone and joint infections, intra-abdominal infections, meningitis, brain abscess, infectious diarrhea and gastrointestinal infections, surgical prophylaxis, and therapy for febrile neutropenic patients.

### Cancer

In one embodiment (e.g., of use in methods of therapy, of use in the manufacture of medicaments, of methods of treatment), the treatment is treatment of cancer, e.g., in a patient.

In one embodiment, the treatment is treatment of: lung cancer, small cell lung cancer, non-small cell lung cancer, throat gastrointestinal cancer, stomach cancer, bowel cancer, colon cancer, rectal cancer, colorectal cancer, thyroid cancer, breast cancer, ovarian cancer, endometrial cancer, prostate cancer, testicular cancer, liver cancer, kidney cancer, renal cell carcinoma, bladder cancer, pancreatic cancer, brain cancer, glioma, sarcoma, osteosarcoma, bone cancer, skin cancer, squamous cancer, Kaposi's sarcoma, melanoma, malignant melanoma, lymphoma, or leukemia.

In one embodiment, the treatment is treatment of:
a carcinoma, for example a carcinoma of the bladder, breast, colon (e.g., colorectal carcinomas such as colon adenocarcinoma and colon adenoma), kidney, epidermal, liver, lung (e.g., adenocarcinoma, small cell lung cancer and non-small cell lung carcinomas), oesophagus, gall bladder, ovary, pancreas (e.g., exocrine pancreatic carcinoma), stomach, cervix, thyroid, prostate, skin (e.g., squamous cell carcinoma);
a hematopoietic tumour of lymphoid lineage, for example leukemia, acute lymphocytic leukemia, B-cell lymphoma, T-cell lymphoma, Hodgkin's lymphoma, non-Hodgkin's lymphoma, hairy cell lymphoma, or Burkett's lymphoma;
a hematopoietic tumor of myeloid lineage, for example acute and chronic myelogenous leukemias, myelodysplastic syndrome, or promyelocytic leukemia;
a tumour of mesenchymal origin, for example fibrosarcoma or habdomyosarcoma;
a tumor of the central or peripheral nervous system, for example astrocytoma, neuroblastoma, glioma or schwannoma;
melanoma; seminoma; teratocarcinoma; osteosarcoma; xenoderoma pigmentoum; keratoctanthoma; thyroid follicular cancer; or Kaposi's sarcoma.

In one embodiment, the cancer is a solid tumour cancer.

### Treatment

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, alleviatiation of symptoms of the condition, amelioration of the condition, and cure of the condition. Treatment as a prophylactic measure (i.e., prophylaxis) is also included. For example, use with patients who have not yet developed the condition, but who are at risk of developing the condition, is encompassed by the term “treatment.”

For example, treatment includes the prophylaxis of infection, reducing the incidence of infection, alleviating the symptoms of infection, etc.

The term “therapeutically-effective amount,” as used herein, pertains to that amount of an active compound, or a material, composition or dosage form comprising an active compound, which is effective for producing some desired therapeutic effect, commensurate with a reasonable benefit/risk ratio, when administered in accordance with a desired treatment regimen.

### Combination Therapies

The term “treatment” includes combination treatments and therapies, in which two or more treatments or therapies are combined, for example, sequentially or simultaneously. For example, the compounds described herein may also be used in combination therapies, e.g., in conjunction with other agents, for example, cytotoxic agents, anticancer agents, etc. Examples of treatments and therapies include, but are not limited to, chemotherapy (the administration of active agents, including, e.g., drugs, antibodies (e.g., as in immunotherapy), prodrugs (e.g., as in photodynamic therapy, GDEPT, ADEPT, etc.); surgery; radiation therapy; photodynamic therapy; gene therapy; and controlled diets.

One aspect of the present invention pertains to a compound as described herein, in combination with one or more additional therapeutic agents.

### Other Uses—Plants

The compounds described herein are also useful, for example, to control (e.g., inhibit) plant growth (e.g., of a seedling; of a plant); to inhibit germination (e.g., plant germination) (e.g., of a seed; of a sprouting seed); as a herbicide; etc. (This utility may be independent of the biochemical mechanism of action described herein.)

Thus, one aspect of the present invention pertains to a method of controlling (e.g., inhibiting) plant growth (e.g., of a seedling; of a plant), comprising contacting a plant (e.g., a living plant, e.g., a growing plant, e.g., a seedling) with an effective amount of a compound as described herein.

Another aspect of the present invention pertains to a method of inhibiting germination (e.g., plant germination) (e.g., of a seed; of a sprouting seed), comprising contacting a seed (or a sprouting seed) with an effective amount of a compound as described herein.

Another aspect of the present invention pertains to use of a compound as described herein as a herbicide.

Another aspect of the present invention pertains to use of a compound as described herein in the manufacture of a herbicidal composition.

The plant (or seed) may be, for example, a food plant (or food plant seed), a crop plant (or crop plant seed), an agricultural crop plant (or agricultural crop plant seed), an agricultural food plant (or agricultural food plant seed), etc.

In one especially preferred embodiment of the above aspects, the compound is selected from Peptide 1 and Peptide 2, and pharmaceutically acceptable salts, amides, esters, solvates, and hydrates thereof.

### Other Uses—Microbes, Bacteria

The compounds described herein are also useful, for example, as a microbicide or anti-microbial agent (e.g., other than in a method of treatment of the human or animal body). (This utility may be independent of the biochemical mechanism of action described herein.)

Thus, one aspect of the present invention pertains to a method of killing a microbe, comprising contacting the microbe with an effective amount of a compound as described herein (e.g., other than in a method of treatment of the human or animal body).

Another aspect of the present invention pertains to use of a compound as described herein as a microbicide or anti-microbial agent (e.g., other than in a method of treatment of the human or animal body), for example, in a method of microbial sterilization.

Another aspect of the present invention pertains to use of a compound as described herein in the manufacture of a microbicidal or anti-microbial agent composition.

The term “microbe,” as used herein, pertains to microscopic organisms, such as: bacteria, fungi, microscopic algae, diatoms, protozoa, and viruses.

The compounds described herein are also useful, for example, as a bactericide or anti-bacterial agent (e.g., other than in a method of treatment of the human or animal body).

Thus, one aspect of the present invention pertains to a method of killing a bacterium (or a method of killing bacteria), comprising contacting the bacterium (or bacteria) with an effective amount of a compound as described herein (e.g., other than in a method of treatment of the human or animal body).

Another aspect of the present invention pertains to use of a compound as described herein as a bactericide or anti-antibacterial agent (e.g., other than in a method of treatment of the human or animal body), for example, in a method of bacterial sterilization.

Another aspect of the present invention pertains to use of a compound as described herein in the manufacture of a bactericidal or anti-antibacterial agent composition.

### Other Uses

The compounds described herein may also be used as cell culture additives to inhibit bacterial cell proliferation, etc.

The compounds described herein may also be used as part of an in vitro assay, for example, in order to determine whether a candidate host is likely to benefit from treatment with the compound in question.

The compounds described herein may also be used as a standard, for example, in an assay, in order to identify other active compounds, other anti-bacterial agents, etc.

### Kits

One aspect of the invention pertains to a kit comprising (a) an active compound as described herein, or a composition comprising an active compound as described herein, e.g., preferably provided in a suitable container and/or with suitable packaging; and (b) instructions for use, e.g., written instructions on how to use or administer the active compound or composition.

The written instructions may also include a list of indications for which the active ingredient is a suitable treatment.

### Routes of Administration

The active compound or pharmaceutical composition comprising the active compound may be administered to a subject by any convenient route of administration, whether systemically/peripherally or topically (i.e., at the site of desired action).

Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### The Subject/Patient

The subject/patient may be a chordate, a vertebrate, a mammal, a placental mammal, a marsupial (e.g., kangaroo, wombat), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human. Furthermore, the subject/patient may be any of its forms of development, for example, a foetus.

In one preferred embodiment, the subject/patient is a human.

### Formulations

While it is possible for the active compound to be administered alone, it is preferable to present it as a pharmaceutical formulation (e.g., composition, preparation, medicament) comprising at least one active compound, as defined above, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents. The formulation may further comprise other active agents, for example, other therapeutic or prophylactic agents.

Thus, the present invention further provides pharmaceutical compositions, as defined above, and methods of making a pharmaceutical composition comprising admixing at least one active compound, as defined above, together with one or more other pharmaceutically acceptable ingredients well known to those skilled in the art, e.g., carriers, diluents, excipients, etc. If formulated as discrete units (e.g., tablets, etc.), each unit contains a predetermined amount (dosage) of the active compound.

The term “pharmaceutically acceptable” as used herein pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

Suitable carriers, diluents, excipients, etc. can be found in standard pharmaceutical texts, for example, Remington's Pharmaceutical Sciences, 18th edition, Mack Publishing Company, Easton, Pa., 1990; and Handbook of Pharmaceutical Excipients, 2nd edition, 1994. The formulations may be prepared by any methods well known in the art of pharmacy.

### Dosage

It will be appreciated by one of skill in the art that appropriate dosages of the active compounds, and compositions comprising the active compounds, can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

### Discussion of the Figures

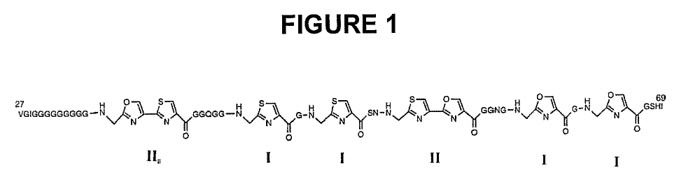

In FIG. 1 there is shown a representation of Microcin B17 (MccB17). The solubility characteristics of this compound are very poor. Solubility of Microcin B17 is about 60 μM in water containing 5% DMSO.

DNA gyrase introduces DNA negative supercoils in the presence of ATP and relaxes them in its absence (Reece & Maxwell, 1991). In vitro, MccB17 has been shown to inhibit both these reactions (Pierrat & Maxwell, 2003). During its catalytic cycle, gyrase produces a double-strand break in the DNA substrate. This break is normally transient but can be trapped by quinolone drugs, CcdB, or Ca²⁺, resulting in a stable complex known as the cleavage complex. When MccB17 was titrated into a mixture of gyrase, relaxed closed-circular DNA, and ATP, followed by incubation for 90 minutes at 37° C., cleaved DNA was produced (Heddle et al., 2001). In the presence of ATP, the IC₅₀ of Microcin B17 is ˜0.9 μM whereas in the absence of the nucleotide, cleavage is only weakly stimulated (Heddle et al., 2001).

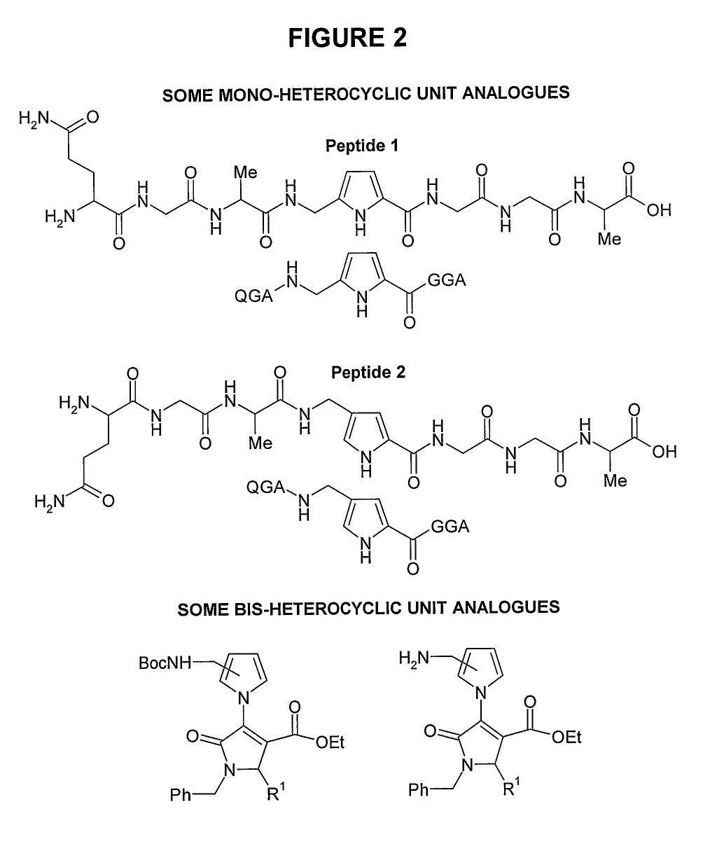

With reference to FIG. 2, it can be seen that Peptide 1 and Peptide 2 are smaller fragments than Microcin B17, soluble respectively in water with 2% DM SO (1000 μM) and water (1000 μM). Syntheses of bis-heterocyclic unit analogues were developed in the laboratory and may find utility upon successful ester group deprotection.

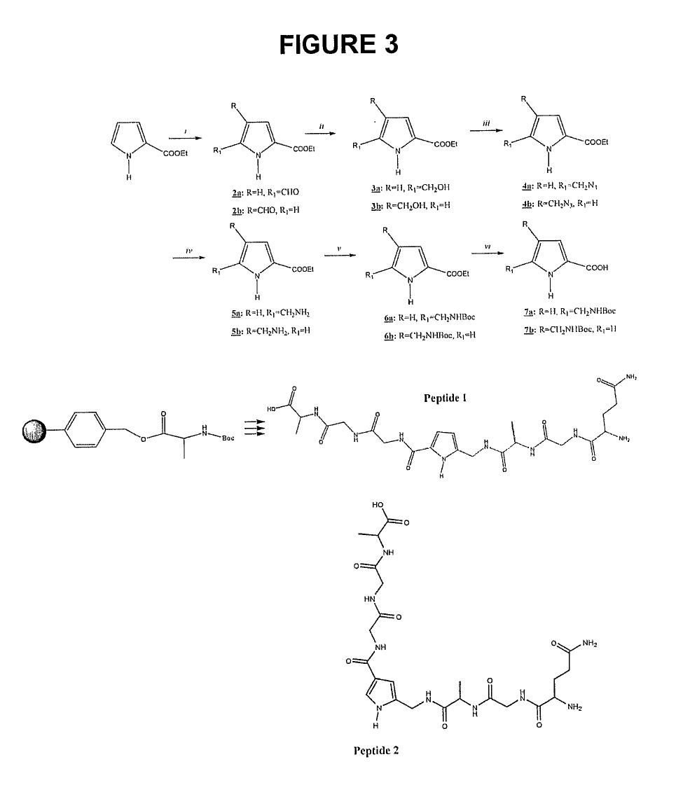

With reference to FIG. 3, a schematic is provided showing solid phase synthesis (Merrifield resin): Gly (x2), B, Gly, Ala and Glu are assembled to yield Peptide 1 following the cycle as follows: deprotection of the N—Boc protective group with a 25% TFA solution in DCM; coupling of an N-protected amino acid using a BOP/HOBt in NMP/DCM (1:1) activation method; cleavage of the peptide using TFMSA/TFA anisole/EDT and purification by ether precipitation.

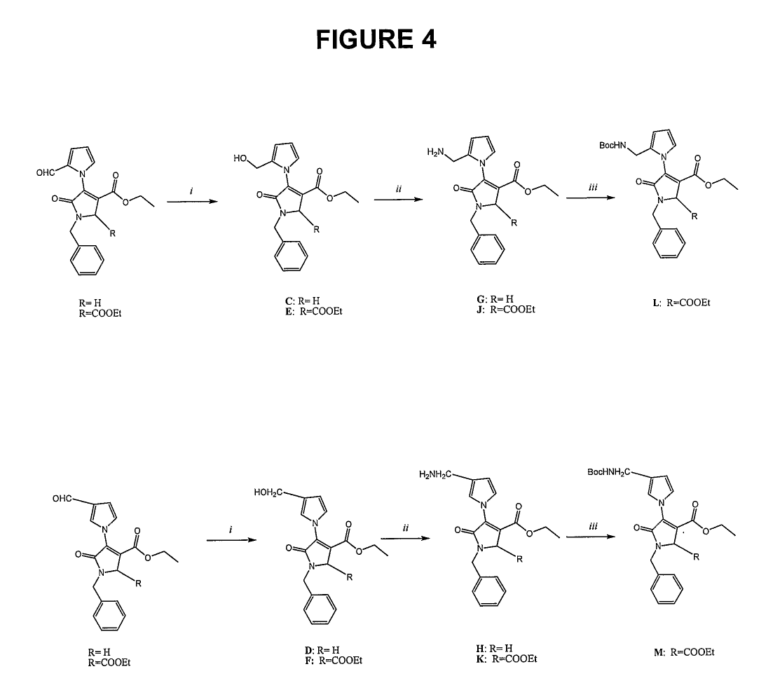

With reference to FIG. 4 there is provided a schematic of synthesis of compounds G, J. L and M: i) NaBH₄ in MeOH, 0° C., ii) P(Ph)₃, DEAD (diethylazidocarboxylate), diphenylphosphorylazide, −20° C. followed by addition of P(Ph)₃, water, 45° C.

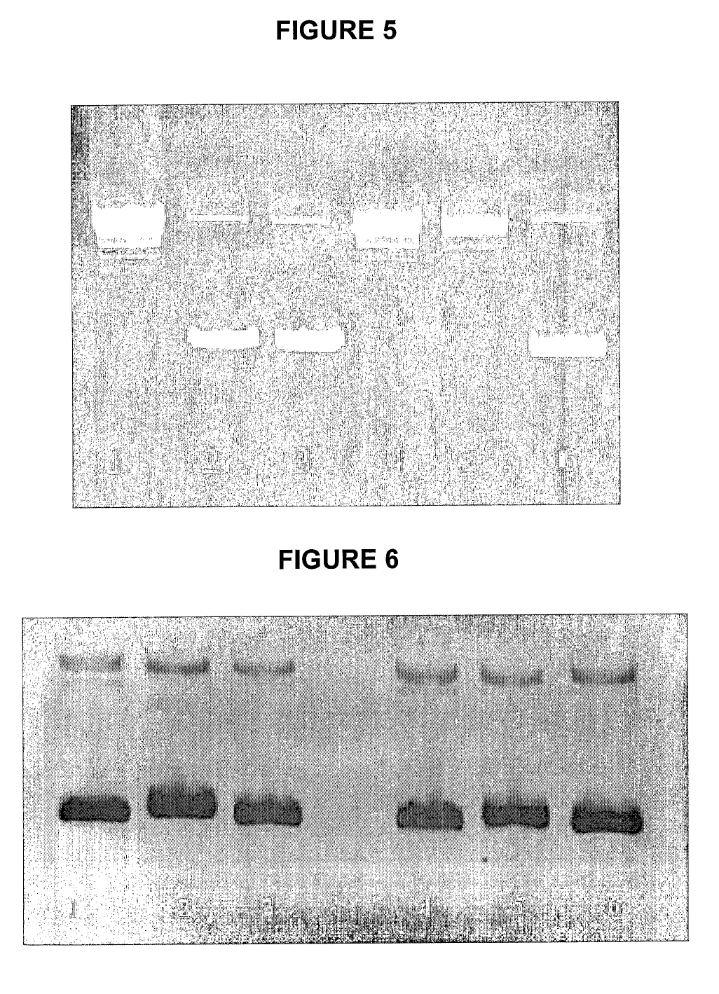

FIG. 5 provides a supercoiling test in which DNA supercoiling reactions were carried out as described by Pierrat & Maxwell (2003), with details provided in example 2 except that the [A₂B₂] was 13.2 nM; reactions were incubated up to 4 hours, and the reactions analysed by electrophoresis: Lane 1: without A₂B₂, Lane 2: DMSO (2%), Lane 3: MccB17 25 μM, Lane 4, 5, 6: Peptide 1 respectively at 200 μM, 100 μM and 50 μM.

FIG. 6 provides a DNA cleavage test in which [A₂B₂]=30 nM, and wherein reactions were incubated up to 1 h. Lane 1, 2, and 3: 30 min, Lane 4, 5 and 6: 1 hour. Lane 1 & 3: no enzyme, Lane 2 & 5: DMSO (2%), Lane 3 & 6: Peptide 1 at 100 μM.

The inventors' conclusions from the foregoing work are that the cleaved DNA-enzyme complex is not stabilised and there is inhibition of supercoiling (IC₅₀<75 μM).

Those skilled in the art will appreciate, based on the present disclosure, that novel compounds according to this invention may be utilized in a wide variety of compositions to achieve desirable anti-bacterial or anti-carcinogenic effects. The enhanced solubility of the compounds according to this invention significantly increases the potential for bioavailability. Further, as evidenced by data provided herein, compounds according to this invention have been unexpectedly found to circumvent resistance to MccB17 in spontaneous mutants. At the same time, utilizing a bacterial strain with a known mutation affecting gyrase susceptibility to MccB17, the inventors have shown that novel compounds according to this invention operate on the same molecular target as MccB17.

Unit dosage forms, treatment regimens and compositions comprising the compounds according to this invention, based on the IC₅₀ as reported herein, may be determined by routine experimentation by those skilled in the art. Further, it will be appreciated that the compounds of this invention provide a basis on which to develop analogues having enhanced activity profiles.

### EXAMPLES

Having generally described this invention, the following examples are provided to ensure that those skilled in the art are enabled to make and use this invention as outlined above. The specifics of these examples should not, however, be construed as limiting on the scope of this invention.

### Example 1

### 4 and 5-formyl-1H-pyrrole-2-carboxylic acid ethyl ester

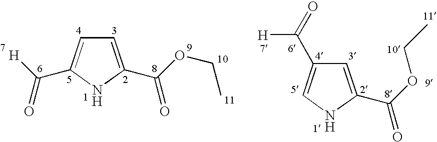

Under N₂, 1.3 equivalents of N,N-dimethylformamide (4.0 mL, 46.8 mmol) and 1.5 equivalents of phosphorus oxychloride (4.2 mL, 54.0 mmol) are mixed at 0° C. (ice bath) and stirred for 10 minutes at room temperature. Subsequently 20 mL of anhydrous 1,2-dichloroethane and 1 equivalent of 1H-pyrrole-2-carboxylic acid ethyl ester (5 g, 36 mmol) in 50 mL of anhydrous 1,2-dichloroethane are added and the solution is stirred at 80° C. for 2 hours. 6 equivalents of ammonium acetate trihydrate are added and the reaction mixture is stirred for 1 hour at 80° C. The solvent is evaporated, the crude oil is dissolved in water and the pH is adjusted to 8 by adding NaHCO₃. The aqueous layer is extracted with dichloromethane and the combined organic layers are washed with brine and water, and dried over CaCl₂ and the solvent was evaporated under reduced pressure to afford a mixture of 4 and 5-formyl-1H-pyrrole-2-carboxylic acid ethyl esters.

Yield: 75% (4.5 g of red oil). ¹H NMR (CD₃OD): 1.28 (t, J_{1→2}=7.1 Hz, 4H, H_{11 and 11′}), 4.25 (q, 2.8H, H_{10 and 10′}), 6.82 (d, J=4.2 Hz, 1H, H_{pyrrolic}), 6.91 (d, J=4.2 Hz, 1H, H_{pyrrolic}), 7.16 (m, 0.3H, H_{pyrrolic}), 7.62 (m, 0.3H, H_{pyrrolic}), 9.63 (s, 1H, H₇), 9.74 (s, 0.3H, H_{7′}), 10.97 (br, 1H, H₁).11.27 (br, 0.3H, H_{1′}). ¹³C NMR (CD₃OD): 14.1 (CH_{3 ester}), 14.3 (CH_{3 ester}), 60.6 (CH_{2 ester}), 60.3 (CH_{2 ester}), 113.9 (C_{pyrrolic}), 115.5 (C_{pyrrolic}), 116.6 (C_{pyrrolic}), 119.7 (C_{pyrrolic}), 125.5 (C_{pyrrolic}), 128.1 (C_{pyrrolic}), 129.4, 135.6 (C_{pyrrolic}), 159.9 (Cₑₛₜₑᵣ) 161.2 (COₑₛₜₑᵣ), 181.4 (CO_{carbaldehyde}) 186.1 (CO_{carbaldehyde}).

### 4-hydroxymethyl-1H-pyrrole-2-carboxylic acid ethyl ester

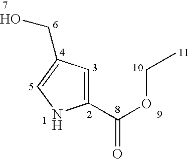

Under N₂, 1.05 equivalents of NaBH₄ (1.2 g, 31.5 mmol) are added to a solution of 4- and 5-formyl-1H-pyrrole-2-carboxylic acid ethyl ester (4.5 g, 30 mmol), in MeOH (50 mL) and stirred at room temperature for 3 hours. The reaction is quenched with 100 mL of water and extracted with ether (2×100 mL). The organic layer is washed with water and dried over MgSO₄. The solvent was evaporated under reduced pressure. The crude oil obtained is purified by column chromatography (1:1 AcOEt:Hexane) to give 2.6 g of a yellow solid and 1.0 g of a brown solid.

Yield: 88% (brown solid). ¹H NMR (CDCl₃): 1.23 (t, J_{11→10}=7.1 Hz, 3H, H₁₁), 4.04 (q, J_{10→11}=7.1 Hz, 2H, H₁₀), 4.45 (s, 2H, H₆) 6.81 (m, 2H, H_{4. and 3}), 10.89 (br, 1H, H₁). ¹³C NMR (CD₃OD): 14.7 (C₁₁), 58.2 (C₆), 61.1 (C₁₀), 115.9 (C_{pyrrolic}), 123.3 (C_{pyrrolic}), 123.7 (C_{pyrrolic}), 126.7 (C_{pyrrolic}), 162.8 (C₈). Microanalysis: C 57.15% (theoretical: 57.02%), H 6.67% (theoretical: 6.51%), N 8.05% (theoretical: 8.28%). Mp=94-95° C. (uncorrected).

### 4-azidomethyl-1H-pyrrole-2-carboxylic acid ethyl ester

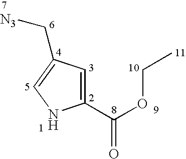

1.5 equivalents of tetrabromomethane are added to a solution of 4-hydroxymethyl 1H-pyrrole-2-carboxylic-acid ethyl ester (1 equivalent; 1.1 g, 6.5 mmol), sodium azide (1.5 equivalents) and triphenylphosphine (1.5 equivalents) in 10 mL of DMF. The reaction mixture is stirred for 1 hour at room temperature. The DMF is evaporated under vacuum and the crude oil obtained is purified by column chromatography (100% Hexane) to give colourless oil.

Yield: 79% (1.0 g of colorless oil). ¹H NMR (CD₃OD): 1.29 (t, J_{11→10}=7.1 Hz, 3H, H₁₁), 4.15 (s, 2H, H₆), 4.30 (q, J_{10→11}=7.1 Hz, 2H, H₁₀), 6.79 (d, J=1.7 Hz, 2H, H_{pyrrolic}). ¹³C NMR (CD₃OD): 14.7 (C₁₁), 48.1 (C₆), 61.3 (C₁₀), 116.2 (C_{pyrrolic}), 120.3 (C_{pyrrolic}), 124.1 (C_{pyrrolic}), 124.4 (C_{pyrrolic}), 162.5 (C₈). Micro Analysis: C 49.20% (theoretical: 49.48%), H 4.32% (theoretical: 4.63%), N 29.15% (theoretical: 28.86%).

### 4-aminomethyl-1H-pyrrole-2-carboxylic acid ethyl ester

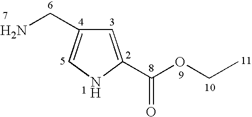

1.5 equivalents of PPh₃ and 12 equivalents of water are added to 4-azidomethyl-1H-pyrrole-2-carboxylic acid ethyl ester (1.0 g, 5.2 mmol) in 20 mL of THF. The reaction mixture is stirred at 45° C. overnight. After completion, THF is evaporated under reduced pressure and the residual oil is purified by column chromatography (100% AcOEt then 100% MeOH) to give a yellow oil (0.8 g).

Yield: 78% (0.7 g of yellow oil). ¹H NMR (CD₃OD): 1.24 (t, J_{10→11}=7.1 Hz, 3H, H₁₁), 3.71 (s, 2H, H₆), 4.17 (q, J_{10→11}=7.1 Hz, 2H, H₁₀), 6.02 (d, J=1.7 Hz, 1H, Hyperbolic), 6.67 (d, J=1.7 Hz, 1H, Hyperbolic). ¹³C NMR (CD₃OD): 14.8 (C₁₁), 38.6 (C₁₂), 61.1 (C₁₀), 109.2 (C_{pyrrolic}), 116.5 (C_{pyrrolic}), 123.2 (C_{pyrrolic}), 133.0 (C_{pyrrolic}), 162.7 (C₈). Micro Analysis: C 57.45% (theoretical: 57.14%), H 6.67% (theoretical: 6.54%), N 16.98% (theoretical: 16.67%). Mp=78-79° C. (uncorrected).

### tert-butyl-(4-(ethoxycarbonyl)-1H-pyrrol-2-yl)methylcarbamate

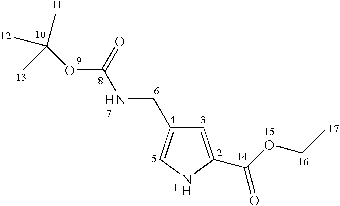

2 equivalents of Na₂CO₃ (0.86 g) and 4 equivalents of NaHCO₃ (1.6 g) are added to a solution of 4-aminomethyl-1H-pyrrole-2-carboxylic acid ethyl ester (4.8 mmol, 0.7 g) in water. The reaction mixture is cooled to 0° C. and 1.1 equivalents of di-tertbutyl dicarbonate (5.6 mmoles, 1.2 g) in 10 mL of THF are added drop wise. The reaction is stirred overnight and subsequently acidified with a 1 M aqueous solution of HCl to pH=1. The solution is extracted with AcOEt (50 mL) and washed with brine twice. Recrystallisation from AcOEt-Hexane gives 0.8 g of a white solid.

Yield: 77%. ¹H NMR (CDCl₃): 1.26 (t, 3H, J_{17→16}=7.1 Hz, H₁₇), 1.40 (s, 9H, H_{11, 12 and 13}), 4.08 (d, 2H, H₆), 4.24 (d, J_{16→17}=6.8 Hz, 2H, H₁₂), 6.05 (d, J=3.16 Hz, 1H, H_{pyrrolic}), 6.65 (d, J=3.20 Hz, 1H, H_{pyrrolic}), 7.2 (t, J=7.32 Hz, 1H, H₇). ¹³C NMR (CDCl₃): 14.5 (C₁₇), 28.2 (C_{11, 12 and 13}), 47.8 (C₆), 61.4 (C₁₆), 80.6 (C₁₀), 112.3 (C_{pyrrolic}), 117.0 (C_{pyrrolic}) 122.6 (C_{pyrrolic}), 134.2 (C_{pyrrolic}), 156.8 (CO₈), 162.7 (CO₁₄). Microanalysis: C % 58.52 (theoretical: 58.19%), H % 7.62 (theoretical: 7.51%), N 10.27% (theoretical: 10.44%). Mp=107-108° C. Mass spectrum (ESI):269.1457 (M+H) (theoretical: 268. 1400).

### tert-butyl-(5-(ethoxycarbonyl)-1H-pyrrol-2-yl)methylcarbamate

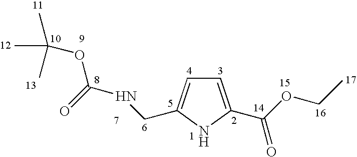

2 equivalents of Na₂CO₃ (0.86 g) and 4 equivalents of NaHCO₃ (1.6 g) are added to a solution of 5-aminomethyl-1H-pyrrole-2-carboxylic acid ethyl ester (4.8 mmol, 0.8 g) in water. The reaction mixture is cooled to 0° C. and 1.1 equivalents of di-tertbutyl dicarbonate (5.6 mmoles, 1.2 g) in 10 mL of THF are added drop wise. The reaction is stirred overnight and subsequently acidified with a 1 M aqueous solution of HCl to pH=1. The white solid product (1.0 g) is removed by filtration and dried under vacuum.

Yield: 63%. ¹H NMR (CDCl₃): 1.26 (t, 3H, J_{17→16}=7.1 Hz, H₁₇), 1.48 (s, 9H, H_{11, 12 and 13}), 4.15 (d, 2H, H₆), 4.29 (d, J_{16→17}=6.8 Hz, 2H, H₁₆), 6.14 (d, J=3.1 Hz, 1H, H_{pyrrolic}), 6.57 (d, J=3.2 Hz, 1H, H_{pyrrolic}), 7.6 (t, J=7.32 Hz, 1H, H₇). ¹³C NMR (CDCl₃): 14.8 (C₁₆), 27.8 (C_{11, 12 and 13}), 41.2 (C₆), 61.4 (C₁₆), 79.6 (C₁₀), 110.3 (C_{pyrrolic}), 118.6 (C_{pyrrolic}) 125.1 (C_{pyrrolic}), 136.8 (C_{pyrrolic}), 152.9 (CO₈), 161.4 (CO₁₄). Microanalysis: C % 58.34 (theoretical: 58.19%), H % 7.78 (theoretical: 7.51%), N 10.56% (theoretical: 10.44%). Mp=115-116° C.

### 4-(tert-butoxycarbonylmethylamino)-1H-pyrrole-2-carboxylic acid (A)

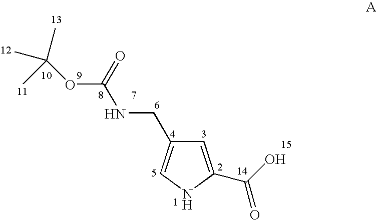

1.2 equivalents of a 1 M aqueous NaOH solution (4.5 mL) are added to a solution of tert-butyl-(4-(ethoxycarbonyl)-1H-pyrrol-2-yl)methylcarbamate (3.8 mmol, 1.0 g) in THF/water (4:1). The reaction mixture is stirred overnight and subsequently 3 equivalents of a 1 M aqueous NaOH solution (11.5 mL) are added over 2 days. The reaction mixture is stirred for 5 days. The solution is acidified with a 1 M aqueous solution of HCl to pH=1, extracted with AcOEt and the crude oil is purified by column chromatography (AcOEt:Hexane 1:1 to AcOEt) to yield 0.8 g of a red oil (A).

A: Yield: 69%. ¹H NMR (CDCl₃): 1.36 (s, 9H, H_{11, 12 and 13}), 4.16 (d, J_{7→8}=5.6 Hz, 2H, H₆), 5.14 (t, J_{7→6}=5.6 Hz, 1H, H₇), 6.45 (d, J=3.2 Hz, 1H, H_{pyrrolic}), 6.92 (d, J=3.2 Hz, 1H, H_{pyrrolic}), 10.89 (br, 1H, H₁), 11.8 (s, 1H, H₁₅). ¹³C NMR (CDCl₃): 27.3 (C_{11, 12 and 13}), 32.8 (C₆), 79.1 (C₁₀), 116.7 (C_{pyrrolic}), 120.1 (C_{pyrrolic}), 125.4 (C_{pyrrolic}), 132.6 (C_{pyrrolic}), 155.2 (CO₈), 173.6 (CO₁₅). Microanalysis: C % 54.78 (theoretical: 54.99%), H % 6.83 (theoretical: 6.71%), N 11.49% (theoretical: 11.66%).

### 5-(tert-butoxycarbonylmethylamino)-1H-pyrrole-2-carboxylic acid (B)

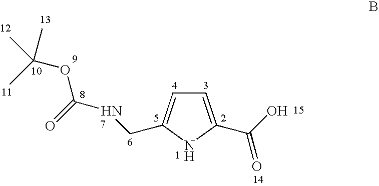

Similarly, 1.2 equivalents of a 1 M aqueous NaOH solution (3.6 mL) are added to a solution of tert-butyl-(5-(ethoxycarbonyl)-1H-pyrrol-2-yl)methylcarbamate (3.0 mmol, 0.8 g) in THF/water (4:1). The reaction mixture is stirred overnight and subsequently 6 equivalents of a 1 M aqueous solution of NaOH (18 mL) are added over 2 days. The reaction mixture is stirred for 5 days. The solution is acidified with a 1 M aqueous solution of HCl to pH=1, extracted with AcOEt and the crude oil is purified by column chromatography (AcOEt:Hexane 1:1 to AcOEt) to yield 0.5 g of an orange oil (B).

B: Yield: 87%. ¹H NMR (CDCl₃): 1.39 (s, 9H, H_{11, 12 and 13}), 4.24 (d, J_{7→8}=5.6 Hz, 2H, H₆), 5.06 (t, J_{8→7}=5.6 Hz, 1H, H₇), 6.03 (d, J=3.2 Hz, 1H, H_{pyrrolic}), 6.85 (d, J=3.20 Hz, 1H, H_{pyrrolic}), 10.01 (br, 1H, H₁), 12.2 (s, 1H, H₁₅). ¹³C NMR (CDCl₃): 28.3 (C_{11, 12 and 13}), 36.4 (C₆), 80.7 (C₁₀), 108.7 (C_{pyrrolic}), 117.1 (C_{pyrrolic}) 121.8 (C_{pyrrolic}), 136.2 (C_{pyrrolic}), 156.7 (CO₈), 171.8 (CO₁₄). Microanalysis: C % 55.25 (theoretical: 54.99%), H % 6.92 (theoretical: 6.71%), N 11.95% (theoretical: 11.66%). Mass spectrum: (M⁺) 240 (10), (M⁺-(CH₃)₃C) 183 (100), 165, 152, 106, 95 (M₁₈₃-CO₂) 139, (M₁₃₉-NH₃) 121, (M₁₂₁-45).

The synthesis of the peptide is presented in the following scheme. For example in the case of Peptide 1, the first amino acid 2 (R₁=CH₃) is anchored to the Merrifield resin 1 using diisopropylcarbodiimide (DIC) and hydroxybenzotriazole (HOBt). In order to ensure a high loading of the resin the condensation step was performed twice. After the removal of the Boc-protective group with TFA (25% in DCM), the immobilized amino acid 3 was coupled to the amino acid 4 (R₁=H) in the presence of Castro's reagent BOP and diisopropylethylamine (DIPEA) to give dimer 5. Stepwise extension of 5 (i.e., Boc deprotection and subsequent coupling) with amino acids 4, B, 4, 2 and 6 (R₁=CH₂CH₂CONH₂) followed by a final Boc deprotection step afforded the immobilized heptamer 7. The cleavage step released the target compound from the solid support. Purification of the crude product by an ether precipitation gives the target molecule in a yield of 48%. LC-MS analysis of the product does not show the presence of a side product and the homogeneity and identity of the heptamer was firmly established by NMR spectroscopy techniques (e.g., COSY, TOCSY, DQF HMQC and NOESY).

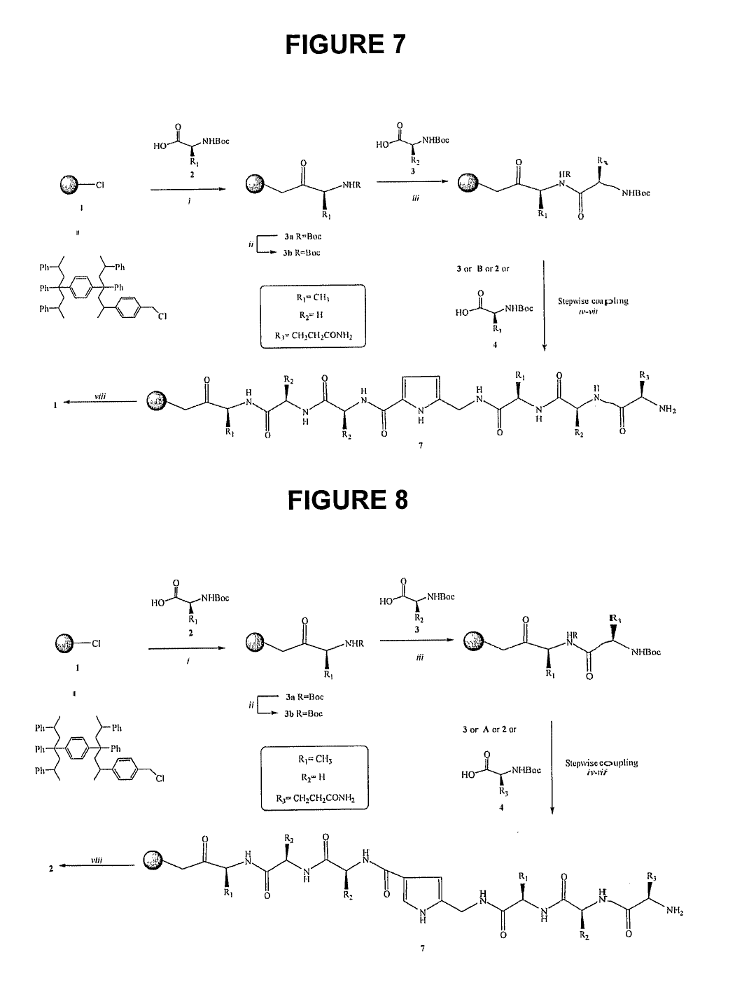

With reference to FIG. 7, there is provided a schematic for the peptide synthesis, in which: i: DIC (5 eq), HOBt (5 eq) in NMP/DCM; ii: Boc-deprotection method; iii: 3 (5 eq), coupling method; iv: Boc-deprotection method, 3 (5 eq), coupling method; v: Boc-deprotection method, number (5 eq), coupling method; vi: Boc-deprotection method, 2 (5 eq), coupling method; vii: Boc-deprotection method, 4 (5 eq), coupling method; viii: Boc-deprotection method, TFMSA conditions. Boc-deprotection conditions: 25% TFA, 1% TIPS in DCM. Coupling method: BOP (5 eq), HOBt (5 eq), DIPEA (6.5 eq).

Peptide 2 is presented in the Scheme outlined in FIG. 8. The first amino acid 2 (R₁=CH₃) is anchored to the Merrifield resin 1 using diisopropylcarbodiimide (BOP) and hydroxybenzotriazole (HOBt). In order to ensure a high loading of the resin the condensation step was performed twice. After the removal of the Boc-protective group with TFA (25% in DCM), the immobilized amino acid 3 was coupled to the amino acid 4 (R₁=H) in the presence of Castro's reagent BOP and diisopropylethylamine (DIPEA) to give dimer 5. Stepwise extension of 5 (i.e., Boc deprotection and subsequent coupling) with amino acids 4, A, 4, 2 and 6 (R₁=CH₂CH₂CONH₂) followed by a final Boc deprotection step afforded the immobilized heptamer 7. The cleavage step released the target compound from the solid support. Purification of the crude product by an ether precipitation gives the target molecule in a yield of 46%. LC-MS analysis of the product does not show the presence of a side product and the homogeneity and identity of the heptamer was firmly established by NMR spectroscopy techniques (e.g., COSY, TOCSY, DQF HMQC and NOESY). With reference to FIG. 8, there is provided a schematic for the peptide synthesis, in which: i: DIC (5 eq), HOBt (5 eq) in NMP/DCM; ii: Boc-deprotection method; iii: 3 (5 eq), coupling method; iv: Boc-deprotection method, 3 (5 eq), coupling method; v: Boc-deprotection method, number (5 eq), coupling method; vi: Boc-deprotection method, 2 (5 eq), coupling method; vii: Boc-deprotection method, 4 (5 eq), coupling method; viii: Boc-deprotection method, TFMSA conditions. Boc-deprotection conditions: 25% TFA, 1% TIPS in DCM. Coupling method: BOP (5 eq), HOBt (5 eq), DIPEA (6.5 eq).

The synthesis was done on a Merrifield resin (0.7 mmol/g on a 50 μmol scale). Freshly prepared stock solutions in NMP of DIC (0.5 M), HOBt (0.5 M), BOP/HOBt (0.5 M/0.5 M) and DIPEA (0.65 M) were used. The building blocks were dissolved either in NMP or DCM, or in mixtures of both, at a concentration of 0.25 M. A 25% (v) TFA solution in DCM containing 1% (v) TIPS was made for the Boc-deprotection reactions.

Anchoring of the first building block: The resin was swollen in DCM for 2 minutes (3×2 mL). The stock solution of the first building block (1 mL, 5 eq was added to the reaction vessel followed by the addition of HOBt (0.5 mL, 5 eq) and DIC (0.5 mL, 5 eq) solutions. The reaction vessel was shaken overnight after which it was drained and subsequently, without rinsing, a second coupling reaction was performed under the same conditions.

Boc-deprotection and elongation of the peptide chain: The Boc group was removed by following four successive 3 minute treatment of the resin with the TFA solution (2 mL), followed by a 4 wash step with DCM (2 mL) and a 4 wash step with NMP (2 mL). For the coupling reaction, the appropriate building block solution (1 mL, 5 eq) was added together with the BOP/HOBt (0.5 mL, 5 eq) and the DIPEA (0.5 mL, 5 eq) solutions. The reaction vessel was shaken for 1 hour after which it was drained and the same coupling reaction was repeated once more. The resin was washed with NMP (4×2 mL) and DCM (4×2 mL). This process was repeated until the desired peptide was obtained.

Final Boc-deprotection, cleavage and purification: The Boc group was cleaved as described above followed by DCM (4×2 mL) and MeOH (4×2 mL) washings. The resin was dried for 24 hours over P₂O₅. The resin was placed in a 25 mL round bottom flask and 75 μL of thioanisole and 25 μL of EDT were added. The mixture was stirred for 10 minutes at room temperature. At 0° C., 750 μL of TFA were added, stirred for 5 minutes and subsequently 25 μL of TFMSA were added drop wise to allow heat to dissipate. The mixture was stirred at room temperature for 1.5 hours. The resin was filtered out and rinsed with TFA (2×1 mL). 5 mL of ether was added and the combined organic layers were concentrated under vacuum (4 times) in order to remove the remaining trace of TFA. Addition of 45 mL of cold ether to the organic phase precipitated the peptide. The peptide was filtered out, rinsed with ether (4×5 mL) and dried over P₂O₅.

Peptide 1: Mass spectrum [M+H⁺]=582.2 [M+Na⁺]=605.8. Fragmentation pattern: M⁺=582.2, (M⁺-H₂O)=564.2, (M⁺-Gln)=418.1, (M⁺-GlyGln)=361.1, (M⁺-AlaGlyGln)=289.9, (M⁺-COAlaGlyGln)=262.1. NMR ¹H (DMSO, 400 MHz): 1.24 (d, 6H, J=7.2 Hz, CH_{3α}Ala×2), 1.93 (dt, 1H, J=10.8 Hz and J=6.1 Hz, H, Gln,), 2.25 (dt, 1H, J=10.6 Hz and J=6.80 Hz, H_{α} Gln), 3.60 (m, 6H, H_{α} Gly), 3.73 (m, 2H, H_{β} Gln), 3.85 (t, 1H, J=7.6 Hz, H_{α} Ala), 4.24 (s, 2H, CH_{2pyr}), 4.37 (t, 1H, J=7.1 Hz, H_{α} Ala), 5.95 (m, 1H, H_{pyrrole}), 6.71 (m, 1H, H_{pyrrole}), 6.93 (br, 1H, H_{δ} Gln), 7.42 (br, 1H, H_{δ} Gln), 8.02 (br, 1H, NH), 8.14 (br, 4H, 4×NH), 8.26 (br, 2H, 2×NH), 8.68 (br, 1H, NH Ala).

Peptide 2: Mass spectrum [M+H⁺]=582.2. Fragmentation pattern: M⁺=582.2, (M⁺-OH)=565.0, (M⁺-H₂O)=564.2, [(M⁺-H₂O)-Ala]=476.1, [(M⁺-(AlaGly+H₂O)]=418.0, [(M⁺-H₂O)-AlaGlyGly)]=361.1, [(M⁺-H₂O)-AlaGlyGlyX_{pyr})]. NMR ¹H (DMSO, 400 MHz): 1.26 (d, 6H, J=7.2 Hz, CH_{3α}Ala×2), 2 31 (m, 2H, H_{α} Gln,), 2.65 (t, 2H, J=6.8 Hz, H_{β} Gln), 3.66 (m, 2H, H_{α} Gly), 3.72 (m, 4H, 2×H_{α} Gly), 3.52 (t, 1H, J=7.56 Hz, H_{α} Ala), 3.82 (s, 2H, CH_{2pyr}), 4.26 (t, 1H, J=7.1 Hz, H_{α} Ala), 6.69 (m, 2H, H_{pyrrole}), 7.80 (br, 2H, H_{β} Gln), 8.0 (br, 1H, NH), 8.20 (br, 5H, 5×NH), 8.58 (br, 1H, NH), 11.14 (br, 1H, NH_{pyrrole}).

### Synthesis of Hydroxymethyl Derivatives C, D, E and F

1.1 equivalents of NaBH₄ are added, under N₂, to a solution of 1 equivalent of ethyl-1-benzyl(−4-(formyl)-1H-pyrrol-1-yl)-2,5-dihydro-5-oxo-1H-pyrrole-3-carboxylate in MeOH (50 mL) and stirred at room temperature for 5 hours. Subsequently the reaction is quenched with water (5 mL). The reaction mixture is extracted with ether (2×50 mL) and the organic phase washed with brine solution, dried over MgSO₄ and evaporated under vacuum. The crude oil obtained is purified through a silica chromatography column (50:50 AcOEt:Hexane) to give the following hydroxymethyl derivatives C, D, E, F in yields of 28-52%.

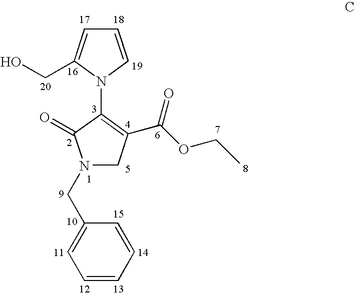

C: Yield: 38% Yellow oil. NMR ¹H (CD₃OD): 1.36 (t, 3H, J_{8→7}=7.1 Hz, H₈), 3.86 (s, 2H, H₅), 4.26 (q, J_{7→8}=7.1 Hz, 2H, H₇), 4.54 (s, 2H, H₂O), 4.65 (s, 2H, H₉), 6.08 (m, 1H, H_{pyrrolic}), 6.26 (m, 1H, H_{pyrrolic}), 6.43 (m, 1H, H_{pyrrolic}), 7.28 (m, 5H, H_{aromatic}). NMR ¹³C (CD3OD): 14.3 (C₈), 45.8 (CH₂), 47.5 (CH₂), 56.4 (C₅), 61.3 (C₇), 109.4 (C_{pyrrolic}), 115.7 (C_{pyrrolic}), 123.5 (C_{pyrrolic}) 124.2 (C_{pyrrolic}), 127.4 (C_{aromatic}), 128.0 (C_{aromatic}), 128.9 (C_{aromatic}), 134.4, 137.6 (C_{aromatic}), 138.2 (C_{aromatic}), 163.3 (CO), 164.8 (CO). Microanalysis: C % 67.36 (theoretical: 67.05%), H % 5.68 (theoretical: 5.92%), N 8.50% (theoretical: 8.23%).

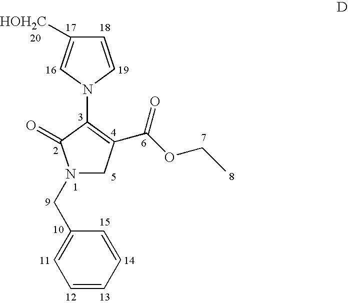

D: Yield: 52% Colourless oil. NMR ¹H (CD₃OD): 1.36 (t, 3H, J_{8→7}=7.1 Hz, H₈), 3.63 (s, 2H, H₅), 4.26 (q, J_{7→8}=7.1 Hz, 2H, H₇), 4.45 (s, 2H, H₉), 4.68 (s, 2H, H₂O), 5.76 (m, 1H, H_{pyrrolic}), 6.41 (m, 1H, H_{pyrrolic}), 6.78 (m, 1H, H_{pyrrolic}), 7.32 (m, 5H, H_{aromatic}). NMR ¹³C (CD3OD): 14.3 (C₈), 48.8 (CH₂), 49.4 (CH₂), 55.4 (C₅), 61.3 (C₇), 111.2 (C_{pyrrolic}), 116.7 (C_{pyrrolic}), 117.8 (C_{pyrrolic}) 118.6 (C_{pyrrolic}), 127.6 (C_{aromatic}), 128.5 (C_{aromatic}), 129.3 (C_{aromatic}), 135.7 (C_{aromatic}), 162.9 (CO), 163.5 (CO). Microanalysis: C % 67.41 (theoretical: 67.05%), H % 5.52 (theoretical: 5.92%), N 8.34% (theoretical: 8.23%).

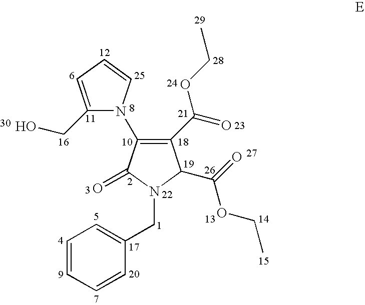

E: Yield: 28% Brown oil. NMR ¹H (CD₃OD): 1.28 (m, 6H, H_{23 and 28}), 4.02 (s, 2H, H₂₉), 4.16 (m, 5H, H_{12, 22 and 27}), 4.57 (s, 1H, H₅), 4.88 (d, 1H, J_{Vic}=15.2 Hz, H₁₂), 5.94 (m, 1H, H_{pyrrolic}), 6.36 (m, 1H, H_{pyrrolic}), 7.56 (m, 1H, H_{pyrrolic}), 7.29 (m, 5H, H_{aromatic}). NMR ¹³C (CD₃OD): 14.4 (CH_{3 ester}), 14.6 (CH_{3 ester}), 42.7 (CH₂), 50.3 (CH_{2 hydroxymethyl}), 61.6 (CH_{2 ester}), 61.8 (CH_{2 ester}) 105.4 (C_{pyrrolic}), 110.3 (C_{pyrrolic}), 116.7 (C_{pyrrolic}) 124.5 (C_{alcenique}), 127.8 (C_{aromatic}), 128.6 (C_{aromatic}), 129.0 (C_{aromatic}), 135.2 (C_{alcenique}), 136.6 (C_{aromatic}), 138.2 (C_{aromatic}), 163.6 (CO), 164.4 (CO). Microanalysis: C % 64.39 (theoretical: 64.07%), H % 5.52 (theoretical: 5.87%), N 6.34% (theoretical: 6.79%).

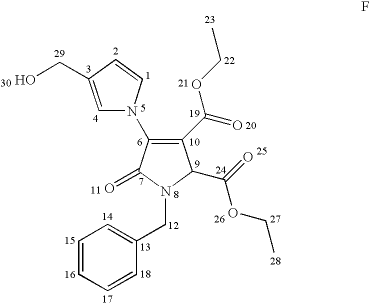

F: Yield: 48% Orange oil. NMR ¹H (CD₃OD): 1.30 (m, 6H, H_{23 and 28}), 4.22 (m, 5H, H_{12, 22 and 27}), 4.67 (s, 2H, H₂₉) 4.94 (d, 1H, J_{Vic}=15.2 Hz, H₁₂), 5.87 (m, 1H, H_{pyrrolic}), 6.57 (m, 1H, H_{pyrrolic}), 7.23 (m, 1H, H_{pyrrolic}), 7.30 (m, 5H, H_{aromatic}). NMR ¹³C(CD₃OD): 14.3 (CH₃ ester), 14.5 (CH_{3 ester}), 43.4 (CH₂), 48.7 (CH_{2 hydroxymethyl}), 61.2 (CH_{2 ester}), 61.6 (CH_{2 ester}) 105.8 (C_{pyrrolic}), 111.0 (C_{pyrrolic}), 117.2 (C_{pyrrolic}) 123.8 (C_{alcenique}), 127.4 (C_{aromatic}), 128.2 (C_{aromatic}), 128.7 (C_{aromatic}), 135.2 (C_{alcenique}), 136.3 (C_{aromatic}), 137.9 (C_{aromatic}), 162.9 (CO), 164.0 (CO). Microanalysis: C % 64.41 (theoretical: 64.07%), H % 6.05 (theoretical: 5.87%), N 7.04% (theoretical: 6.79%).

### Synthesis of the Aminomethyl Derivatives G, H, J and K

Four equivalents of P(Ph)₃, DEAD (diethylazocarboxylate) and diphenylazidophosphorus are added, under N₂ at −20° C., to a solution of 1 equivalent of ethyl-1-benzyl-2,5-dihydro (-4-(hydroxymethyl)-1H-pyrrol-1-yl)-5-oxo-1H-pyrrole-3-carboxylate in distilled THF (50 mL). The solution is stirred for 6 hours at this temperature and allowed to warm to room temperature overnight. Subsequently 1.5 equivalents of P(Ph)₃ and 10 equivalents of water are added and the reaction mixture is stirred at 45° C. for 4 hours. The solvent is evaporated under vacuum and the crude oil is purified by column chromatography (gradient of eluant from 25/75 ethyl acetate-hexane to 75/25 ethyl acetate-hexane) to give the aminomethyl derivatives, G, H, J, K, in yields of 35-55%.

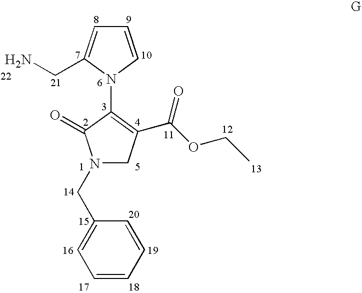

G: Yield: 56% Yellow oil. NMR ¹H (CDCl₃): 1.30 (t, 3H, J_{13→12}=7.1 Hz, H₁₃), 3.72 (s, 2H, CH₂), 3.89 (s, 2H, CH₂), 4.28 (q, J_{12→13}=7.1 Hz, 2H, H₁₂), 4.54 (s, 2H, H₁₄), 4.81 (br, 2H, H₂₂), 5.94 (m, 1H, H_{pyrrolic}), 6.36 (m, 1H, H_{pyrrolic}), 6.56 (m, 1H, H_{pyrrolic}), 7.36 (m, 5H, H_{aromatic}). NMR ¹³C (CDCl₃): 14.3 (C₁₃), 38 (C₂₁), 42.7 (CH₂), 49.3 (CH₂), 61.3 (C₁₂), 106.8 (C_{pyrrolic}), 108.3 (C_{pyrrolic}), 115.7 (C_{pyrrolic}) 123.5 (C_{alcenique}), 127.4 (C_{aromatic}), 128.0 (C_{aromatic}), 128.9 (C_{aromatic}), 134.4 (C_{alcenique}), 137.6 (C_{aromatic}), 138.2 (C_{aromatic}), 164.6 (CO), 165.4 (CO). Microanalysis: C % 67.49 (theoretical: 67.24%), H % 6.47 (theoretical: 6.24%), N 12.12% (theoretical: 12.38%).

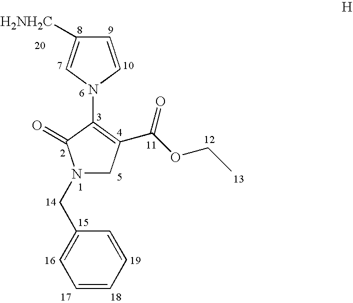

H: Yield: 34% Brown oil. NMR ¹H (CD₃OD): 1.36 (t, 3H, J_{13→12}=7.1 Hz, H₁₃), 3.81 (s, 2H, H₅), 4.16 (s, 2H, H₂₀), 4.31 (q, J_{12→13}=7.1 Hz, 2H, H₁₂), 4.56 (s, 2H, H₁₄), 5.86 (m, 1H, H_{pyrrolic}), 6.32 (m, 1H, H_{pyrrolic}), 6.86 (m, 1H, H_{pyrrolic}), 7.32 (m, 5H, H_{aromatic}). NMR ¹³C (CD3OD): 14.3 (C₁₃), 47.6 (CH₂), 48.2 (CH₂), 52.4 (C₇), 60.4 (C₁₂), 111.6 (C_{pyrrolic}), 116.2 (C_{pyrrolic}), 117.4 (C_{pyrrolic}) 119.3 (C_{pyrrolic}), 127.0 (C_{aromatic}), 127.8 (C_{aromatic}), 128.9 (C_{aromatic}), 134.9 (C_{aromatic}), 161.9 (CO), 162.9 (CO). Microanalysis: C % 67.54 (theoretical: 67.24%), H % 6.39 (theoretical: 6.24%), N 12.65% (theoretical: 12.38%).

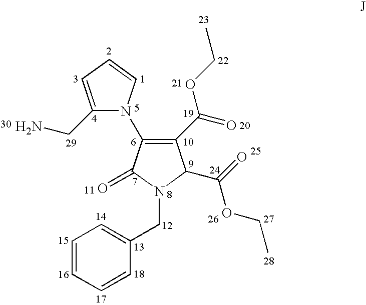

J: Yield: 45% Orange oil. NMR ¹H CD₃OD): 1.32 (m, 6H, H_{23 and 28}), 3.78 (s, 2H, H₂₉), 4.16 (m, 5H, H_{12, 22 and 27}), 4.56 (s, 1H, H₅), 4.96 (d, 1H, J_{vic}=15.2 Hz, H₁₂), 5.94 (m, 1H, H_{pyrrolic}), 6.36 (m, 1H, H_{pyrrolic}), 7.32 (m, 5H, H_{aromatic}), 7.56 (m, 1H, H_{pyrrolic}). NMR ¹³C (CD₃OD): 14.3 (CH_{3 ester}), 14.5 (CH_{3 ester}), 43.5 (CH₂), 44.3 (CH_{2 hydroxymethyl}), 61.0 (CH_{2 ester}), 61.2 (CH_{2 ester}) 105.8 (C_{pyrrolic}), 110.2 (C_{pyrrolic}), 115.2 (C_{pyrrolic}) 123.1 (C_{alcenique}), 127.0 (C_{aromatic}), 128.1 (C_{aromatic}), 128.7 (C_{aromatic}), 135.7 (C_{alcenique}), 137.0 (C_{aromatic}), 137.8 (C_{aromatic}), 164.6 (CO), 166.1 (CO). Microanalysis: C % 64.45 (theoretical: 64.22%), H % 6.26 (theoretical: 6.12%), N 10.47% (theoretical: 10.21%).

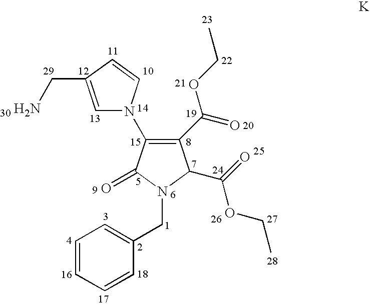

K: Yield: 52% Brown oil. NMR ¹H (CD₃OD): 1.28 (m, 6H, H_{23 and 28}), 4.16 (m, 7H, H_{12, 29, 22 and 27}), 4.56 (s, 1H, H₅), 5.08 (d, 1H, J_{vic}=15.2 Hz, H₁₂), 6.05 (m, 1H, H_{pyrrolic}), 6.56 (m, 1H, H_{pyrrolic}), 7.34 (m, 5H, H_{aromatic}), 7.50 (m, 1H, H_{pyrrolic}). NMR ¹³C (CD₃OD): 14.4 (CH₃ ester), 14.6 (CH_{3 ester}), 42.7 (CH₂), 40.5 (CH_{2 hydroxymethyl}), 61.6 (CH_{2 ester}), 61.8 (CH_{2 ester}) 105.2 (C_{pyrrolic}), 110.9 (C_{pyrrolic}), 116.1 (C_{pyrrolic}) 124.7 (C_{alcenique}), 127.8 (C_{aromatic}), 128.6 (C_{aromatic}), 129.0 (C_{aromatic}), 134.9 (C_{alcenique}), 136.6 (C_{aromatic}), 138.2 (C_{aromatic}), 163.9 (CO), 165.4 (CO). Microanalysis: C % 64.56 (theoretical: 64.22%), H % 5.96 (theoretical: 6.12%), N 10.43% (theoretical: 10.21%).

### tert-butyl (1-(4,5-di(ethoxycarbonyl)-1-benzyl-2,5-dihydro-2-oxo-1H-pyrrol-3-yl)-1H-pyrrol-2-yl)methylcarbamate (L), and

### tert-butyl (1-(4,5-di(ethoxycarbonyl)-1-benzyl-2,5-dihydro-2-oxo-1H-pyrrol-3-yl)-1H-pyrrol-3-yl)methylcarbamate (M)

1.2 equivalents of di-tert-butyl dicarbonate is added to a solution of 1 equivalents of diethyl-4-(aminomethyl-1H-pyrrol-1-yl)-1-benzyl-2,5-dihydro-5-oxo-1H-pyrrole-2,3-dicarboxylate in distilled CH₂Cl₂ (50 mL). The reaction mixture is stirred at room temperature overnight and the organic layer is washed with water, dried over CaCl₂ and evaporated under vacuum to give oil which is purified by column chromatography (hexane-ethyl acetate) to give the following N—Boc protected derivatives L and M.

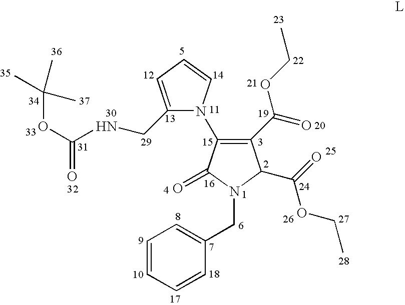

L: Yield: 45% Colourless oil. NMR ¹H (CD₃OD): 1.28 (m, 6H, H_{23 and 28}), 1.39 (s, 9H, H_{35, 36 and 37}), 3.96 (s, 2H, H₂₉) 4.40 (m, 5H, H_{12, 22 and 27}), 4.62 (s, 1H, H₅), 4.87 (d, 1H, J_{vic}=15.2 Hz, H₁₂), 5.99 (m, 1H, H_{pyrrolic}), 6.28 (m, 1H, H_{pyrrolic}), 7.38 (m, 5H, H_{aromatic}), 7.45 (m, 1H, H_{pyrrolic}). NMR ¹³C (CD₃OD): 14.4 (CH_{3 ester}), 14.6 (CH_{3 ester}), 27.4 (C_{35, 36, 37}), 39.7 (CH₂), 45.7 (CH₂), 55.1 (CH), 61.6 (CH_{2 ester}), 61.8 (CH_{2 ester}), 80.5 (C₁), 105.8 (C_{pyrrolic}), 111.8 (C_{pyrrolic}), 116.7 (C_{pyrrolic}) 125.1 (C_{alcenique}), 128.2 (C_{aromatic}), 128.9 (C_{aromatic}), 129.4 (C_{aromatic}), 135.4 (C_{alcenique}), 137.2 (C_{aromatic}), 137.9 (C_{aromatic}), 156.7 (CO_{t-Boc}), 162.9 (CO), 166.4 (CO). Microanalysis: C % 63.56 (theoretical: 63.39%), H % 6.12 (theoretical: 6.50%), N 8.46% (theoretical: 8.21%).

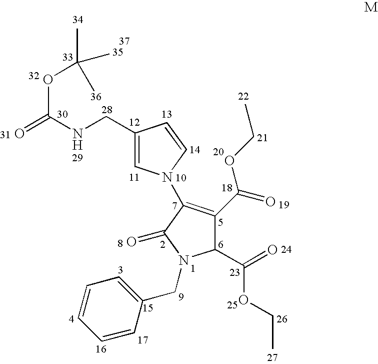

M: Yield: 65% Colourless oil. NMR ¹H (CD₃OD): 1.30 (m, 6H, H_{23 and 28}), 1.42 (s, 9H, H_{35, 36 and 37}), 4.40 (m, 7H, H_{12, 29, 22 and 27}), 4.78 (s, 1H, H₅), 4.94 (d, 1H, J_{vic}=15.2 Hz, H₁₂), 6.03 (m, 1H, H_{pyrrolic}), 6.35 (m, 1H, H_{pyrrolic}), 7.32 (m, 5H, H_{aromatic}), 7.40 (m, 1H, H_{pyrrolic}). NMR ¹³C (CD₃OD): 14.3 (CH_{3 ester}), 14.6 (CH_{3 ester}), 28.0 (C_{35, 36, 37}), 42.1 (CH₂), 45.7 (CH₂), 55.8 (CH), 61.5 (CH_{2 ester}), 61.9 (CH_{2 ester}), 81.2 (C₃₄), 106.8 (C_{pyrrolic}), 112.4 (C_{pyrrolic}), 118.1 (C_{pyrrolic}) 124.2 (C_{alcenique}), 128.4 (C_{aromatic}), 129.2 (C_{aromatic}), 129.7 (C_{aromatic}), 136.7 (C_{alcenique}), 136.8 (C_{aromatic}), 138.0 (C_{aromatic}), 155.7 (CO_{t-Boc}), 164.9 (CO), 166.9 (CO). Microanalysis: C % 63.47 (theoretical: 63.39%), H % 6.42 (theoretical: 6.50%), N 8.39% (theoretical: 8.21%).

### Example 2

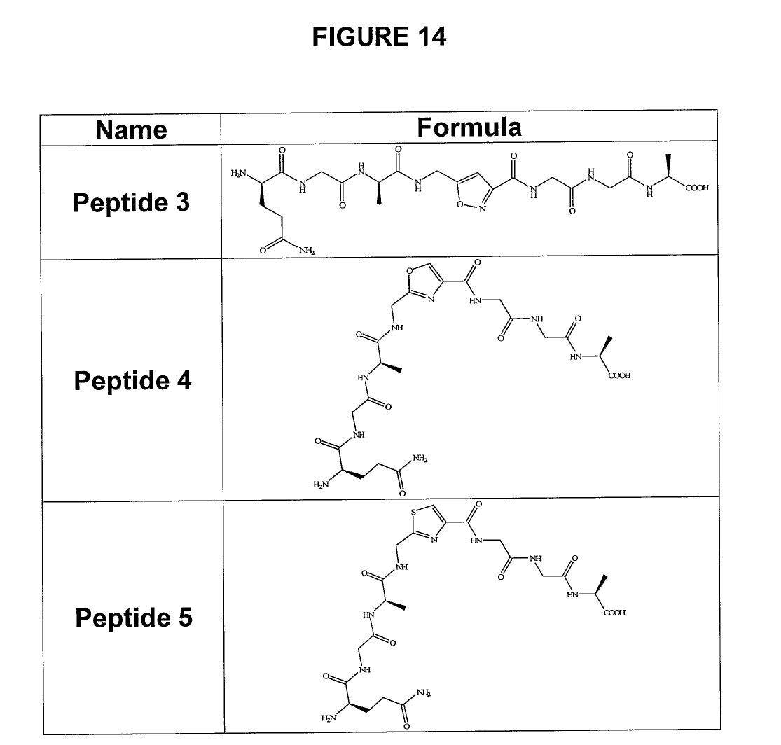

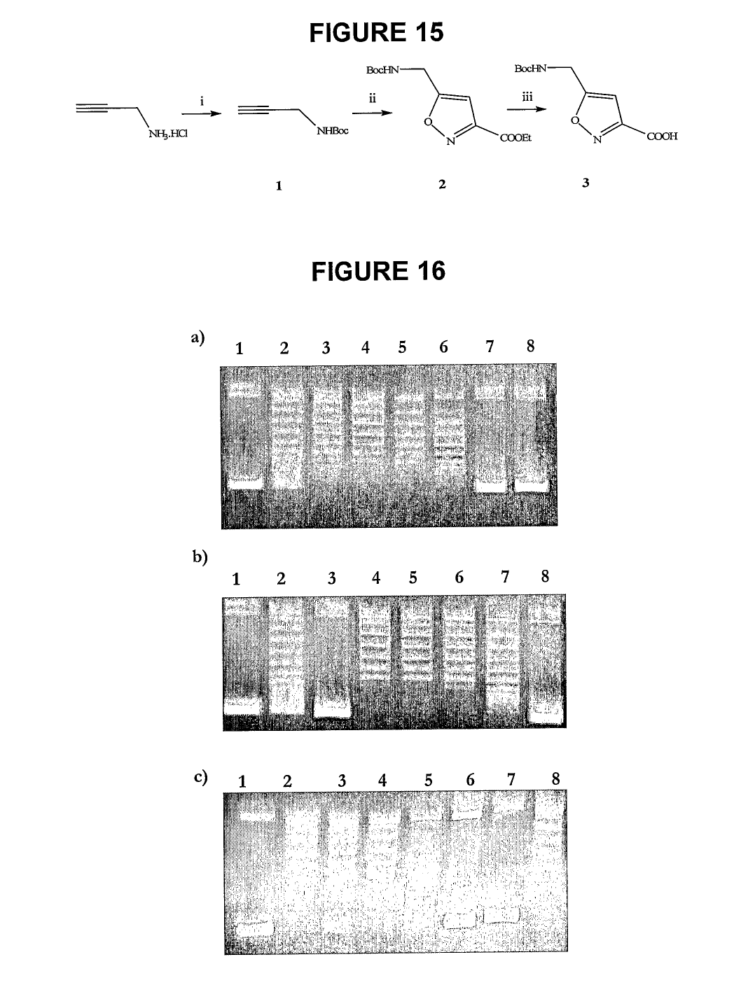

Peptide 3, Peptide 4, and Peptide 5 (shown in FIG. 14) were synthesised using methods analogous to those described above using, respectively, analogue C, an oxazole building block, and a thiazole building block. The synthesis of analogue C is summarized in FIG. 15 in which: i: (Boc)₂O, NaOH in THF, ii: ethylchlorohydroxyimino-acetate, NEt₃ in diethylether, iii: LiOH in H2O/THF (1:4). The oxazole building block and the thiazole building block were synthesised using methods described in Videnov et al., 1996.

### N—Boc Propargyl amine (1)

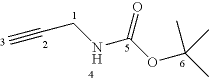

1 equivalent of an 1M aqueous solution of NaOH (109 mL) and 1.2 equivalents of di-tert-butyl carbonate (28.5 g, 130 mmol) are added to a solution of propargylamine hydrochloride (10 g, 109 mmol) in THF (340 mL). The reaction mixture is stirred overnight and extracted with AcOEt (2×200 mL). The combined organic layers were washed with brine, dried over MgSO₄ and concentrated under vacuum. The residual oil is purified by chromatography column (AcOEt: hexane, 35:65) to afford 13.5 g of a white solid.

Yield: 80%. NMR ¹H (CDCl₃, 400 MHz): 1.29 (s, 9H, (CH₃)₃C), 2.01 (s, 1H, H₃), 3.75 (d, J=6.8 Hz, 2H, 2-H₁), 5.16 (t, J=6.8 Hz, 1H, H₄). NMR ¹³C(CDCl₃, 100 MHz): 27.9 ((CH₃)₃C), 29.9 (C₁), 80.0 (C₆), 84.8 (C₃), 155.1 (C₅). Microanalysis: C % 61.76 (theoretical: 61.91%), H % 8.53 (theoretical: 8.44%), N 8.76% (theoretical: 9.02%). MP=37-38° C.

### tert-butyl (3-ethoxycarbonyl)-isoxazol-5-yl)-methylcarbamate (2)

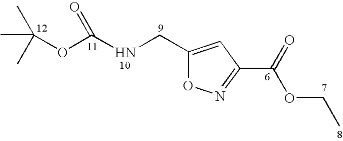

At 0° C., 1 equivalent of ethyl-2-chloro-2-(hydroximino)acetate (0.76 g, 5.0 mmol) and 1 equivalent of NEt₃ (0.7 mL, 5.0 mmol) are added to a solution of N—Boc propargylamine (1 g, 6.45 mmol) in diethylether (50 mL). The reaction mixture is stirred 24 hours. NEt₃.HCl is filtered off and the solvent is concentrated under vacuum. The residual oil obtained is purified by column chromatography (AcOEt: Hexane, 1:9 to 1:4) to afford 0.4 g of a yellow solid.

Yield: 29.6%. NMR ¹H (CDCl₃, 400 MHz): 1.19 (t, J=7.3 Hz, 3H, 3-H₈), 1.38 (s, 9H, (CH₃)₃C), 4.05 (q, J=7.3 Hz, 2H, 2-H₇) 4.40 (d, J=6.4 Hz, 2H, 2-H₉), 5.12 (t, J=6.4 Hz, 1H, H₁₀), 6.53 (s, 1H, H₄). NMR ¹³C (CDCl₃, 100 MHz): 14.0 (C₈) 28.2 ((CH₃)₃C), 36.5 (C₉), 62.1 (C₇) 80.4 (C₁₂), 102.4 (C₄), 155.4 (C₁₁), 156.4 (C₃), 159.7 (C₄), 171.8 (C₆). Microanalysis: C % 53.05 (theoretical: 53.33%), H % 6.82 (theoretical: 6.71%), N 10.19% (theoretical: 10.36%). MP=59-60° C.

### tert-butyl (3-carboxylic acid)-isoxazol-5-yl)-methylcarbamate (3)

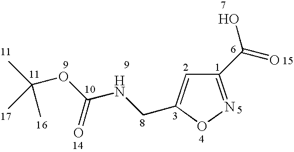

1.2 equivalents of LiOH (3.12 mL, 1 M) are added to a solution of isoxazole 2 (0.4 g, 1.56 mmol) in THF (7 mL) and stirred 2 hours. The reaction mixture is acidified to pH=3 with a 1 M aqueous solution of HCl and extracted with AcOEt (2×15 mL). The organic phases are washed with brine dried upon MgSO₄ and concentrated under vacuum. The residual oil is purified by column chromatography (AcOEt:hexane, 1:1 to AcOEt) to afford 0.294 g of an orange oil.

Yield: 80%. NMR ¹H (CDCl₃, 400 MHz): 1.40 (s, 9H, (CH₃)₃C), 4.29 (d, J=6.4 Hz, 2H, 2-H₈), 5.32 (t, J=6.4 Hz, 1H, H₉), 6.36 (s, 1H, H₄). NMR ¹³C (CDCl₃, 100 MHz): 28.7 ((CH₃)₃C), 36.9 (C₈), 80.7 (C₁), 102.8 (C₄), 157.7 (C₁₀), 162.7 (C₃), 166.6 (C₅), 172.4 (C₆). Mass spectrum: (70e), m/z negative mode: 287.0 (100), 241 (20) [M−H], 197 [M-CO₂] (55), 123 [M-(CH₃)₃OH] (15), 81 (5). (*Formic adduct of the isoxazole). Microanalysis: C % 49.34 (theoretical: 49.58%), H % 5.72 (theoretical: 5.83%), N 11.24% (theoretical: 11.58%).

### Peptide 3

Yield: 11 mg (16 mg) 68.7%. LC-MS: tᵣ=1,3 (584.4), tᵣ=2,3 (584.4). Fragmentation pattern: (M⁺-NH₃)=567.1 (46), (M⁺-H₂0)=566.0 (100), [(M⁺-CO)—NH₃)=549.2 (10), [(M⁺-CO)—H₂O)]=548.4 (45), [(M⁺-Ala)-NH₃)]=479.4 (20), [(M⁺-Ala)-H₂O)]=478.0 (80), [(M⁺-AlaGly)-H₂O)]=421.0 (10), (M⁺-GlnAlaGly)=326.0 (10), [(M⁺-GlnAlaGly)-CH₂NH)]=297.0 (40), (M⁺-AlaGlyGlyX_{isoxazole})=257 (10).

### Peptide 4

Yield: 16.5 mg (29 mg) 58.0%. Mass spectrum (m/z): [M⁺H⁺]=584.7 (40), [(M-X_{oxazole})+Na⁺]=460 (10), 326.3 (20), 249.2 (25), 168.0 (100). LC-MS: tᵣ=1,3 (584.3), tᵣ=5,3 (584.3). Fragmentation pattern: M⁺=584.3 (100), (M⁺-NH₃)=567.0 (40), (M⁺-H₂O)=566.1 (50), (M⁺-Ala)=495 (100), [(M⁺-Ala)-NH₃]=478 (35), [(M⁺-Ala)-H₂0]=477 (45), [(M⁺-AlaGly)-H₂0]=421.0 (25), (M⁺-GlnGly)=398.1 (15), [(M⁺-GlnGly)-NH₃]=381.0 (2), [M⁺-GlnGlyAla)=327.0 (5), [(M⁺-GlnGlyAlA)-H₂0]=309.2 (10), [M⁺-AlaGlyGlyX_{oxazole}]=257.1 (2), [(M⁺-AlaGlyGlyX_{oxazole})—H₂0]=238.9 (2). NMR ¹H (DMSO, 400 MHz): 1.25 (d, 6H, J=7.2 Hz, CH_{3α}Ala×2), 1.92 (m, 2H, H_{α} Gln), 2.21 (t, 2H, J=6.8 Hz, Hp Gln), 3.61 (m, 4H, 3×H_{α} Gly), 3.85 (d, 2H, J=7.56 Hz, CHa oxazole), 3.82 (s, 2H, CH_{2pyr}), 4.21 (t, 1H, J=7.1 Hz, H_{α} Ala), 4.42 (t, 1H, J=7.1 Hz, H_{α} Ala), 6.96 (s, 1H, H₅ Gln), 7.41 (s, 2H, H_{δ} Gln), 8.13 (br, 4H, N⁵H, N⁶H N⁷H and CH oxazole), 8.27 (s, 1H, CH oxazole), 8.57 (s, 1H, N⁴H), 8.66 (br, 2H, N²H), 12.6 (br, 1H, COOH).

### Peptide 5

Yield: 17.2 mg (29 mg) 60%. LC-MS: tᵣ=1,3 (584), tᵣ=7,3 (584). Fragmentation pattern: M⁺=600 (100), (M⁺-NH₃)=583.2 (50), (M⁺-H₂O)=582.1 (60), [(M⁺-Ala)-NH₃]=495.0 (10), [(M⁺-Ala)-H₂0]=494.0 (40), [(M⁺-AlaGly)-NH₃]=438.1 (10), [(M⁺-AlaGly)-H₂0]=437.0 (60), [(M⁺-AlaGlyCO)—H₂0]=409.0 (15), [(M⁺-GlnAlaGly)]=344.0 (5), [(M⁺-GlnAlaGly)-NH₃]=327.3 (5). NMR ¹H (DMSO, 400 MHz): 1.19, (d, 3H, J=7.2 Hz, CH_{3α}Ala₇×2), 1.25 (d, 3H, J=7.2 Hz, CH_{3α}Ala₃), 1.92 (m, 2H, H_{α}, Gln), 2.22 (t, 2H, J=6.8 Hz, H_{β} Gln), 3.58 (m, 6H, 3×H_{α} Gly), 3.84 (d, 2H, J=7.56 Hz, CH₂ thiazole), 4.17 (t, 1H, J=7.1 Hz, H, Ala₃), 4.42 (t, 1H, J=7.1 Hz, H_{α} Ala₇), 6.95 (s, 1H, H₅ Gln), 7.41 (s, 1H, H Gln), 8.14 (br, 5H, N²H, N³H, N⁴H, N⁵H, N⁶H and N⁷H), 8.64 (s, 1H, CH thiazole).

### Example 3

### Supercoiling In Vitro Assay

GyrA and GyrB were added to a solution containing 35 mM Tris.HCl (pH, 7.5), 24 mM KCl, 4 mM MgCl₂, 1.8 mM spermidine, 6.5% glycerol, 0.36 mg/mL BSA, 9 μg/mL tRNA, 5 mM DTT, 2 mM ATP and 24 nM relaxed pBR322 DNA. The reaction contained A₂B₂ dimer at 13.2 nM and also Microcin B17 at 25 μM or Peptide 1 at varying concentrations (respectively 50 μM, 100 μM and 200 μM) and the amount of DMSO was kept constant at (3.33% for Microcin B17 and 2% for Peptide 1). The reactions were incubated at 25° C., and at each time point 30-μl aliquots were quenched with 1 μL of 10% SDS. An equal volume of chloroform/isoamyl alcohol (24:1) and a half volume of loading buffer STEB (40% sucrose/100 mM Tris.HCl, pH 7.5/100 mM EDTA/2 mg/ml bromophenol blue) were added, and the mixtures then were vortexed and centrifuged for 1 min at 13,000 rpm. The aqueous phase was loaded onto 1% agarose, TAE (40 mM Tris-acetate, 1 mM EDTA) gels and were run at either 30 V overnight in TAE (in the cold room) or 70 V for 2.5 hours. The gels were stained for about 20 min in TAE containing 1 μg/ml ethidium bromide followed by destaining in multiple washes of TAE.

### Cleavage In Vitro Assay

GyrA and GyrB were added to solutions containing 35 mM Tris.HCl (pH, 7.5), 24 mM KCl, 4 mM MgCl₂, 1.8 mM spermidine, 6.5% glycerol, 0.36 mg/mL BSA, 9 μg/mL tRNA, 5 mM DTT, 2 mM ATP and 10 nM relaxed pBR322 DNA. The reaction contains dimer A₂B₂ and also Microcin B17 at 25 μM (data not shown) or Peptide 1 at 100 μM; the amount of DMSO was kept constant at 3.33%. The reactions were incubated at 25° C., and at each time point 30-μl aliquots were quenched with 1 μL of 10% SDS and 2 μL of 2 mg/ml proteinase K. Quenched aliquots were kept on ice until the experiment was completed, at which time they were incubated at 37° C. for half an hour. An equal volume of chloroform/isoamyl alcohol (24:1) and a half volume of loading buffer STEB (40% sucrose/100 mM Tris HCl, pH 7.5/1 mM EDTA/2 mg/ml bromophenol blue) were added, and the mixtures then were vortexed and centrifuged for 1 min at 13,000 rpm. The aqueous phase was loaded onto 1% agarose, TAE (40 mM Tris-acetate, 1 mM EDTA) gels that contained 1 μg/ml ethidium bromide and were run at either 30 V overnight in TAE containing 1 μg/ml ethidium bromide (in the cold room) or 70 V for 2.5 hours in TAE containing 1 μg/ml ethidium bromide.

### Example 4

### In Vivo Haloassay

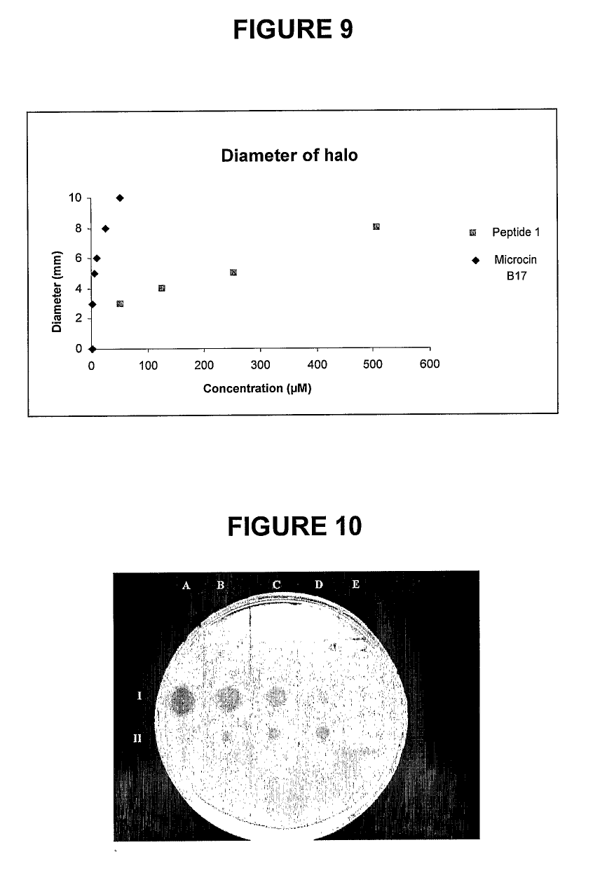

In vivo activity was tested using the bioassay developed by Sinha Roy, 1999, with some modifications. The Microcin-sensitive E. coli DH5α was used as the indicator strain. Lawns of cells were prepared by spreading 100 μL of a 10 mL LB culture grown at 37° C. overnight mixed with 3 mL of LSS, on LB+agar (10 g/L) plates. Aliquots of Microcin B17 in 10% DMSO at different concentrations and aliquots of Peptide 1 in 10% DMSO (see Table 1 below), were spotted on the lawns. The plates were incubated at 37° C. overnight and halos of growth inhibition were qualitatively analyzed by determining their diameters (see FIG. 9).

TABLE 1

| Microcin B17 | Peptide 1 |
|---|---|
| (μM) | (μM) |
| 50 | 508 |
| 25 | 254 |
| 10 | 125 |
| 5 | 50 |
| 2.5 | |
| 1 | |

Concentrations were: Microcin B17 50 μM, 25 μM, 10 μM, 5 μM, 2.5 μM, 1 μM and Peptide 1 50 μM, 125 μM, 254 μM and 508 μM. Peptide 1 is active at 50 μM whereas Microcin B17 has the same activity at 2.5 μM. Spots indicate development of resistance colonies which are only seen with Microcin B17 and not Peptide 1, see FIG. 10.

### Unwinding Assay

Peptide 1-induced DNA unwinding was examined using a DNA topoisomerase 1-based assay (Pommier et al., 1987). Other compounds like Ethidium Bromide (EtBr), MccB17 and Ciprofloxacin (CFX) were also tested as controls. In this assay, negatively supercoiled pBR322 was relaxed by topoisomerase I in the presence or the absence of the putative DNA intercalative compound. Following relaxation, the test compound is removed and, if intercalation occurred, the rewinding of the DNA into its negatively supercoiled form can be observed. Sample reactions were performed in the same buffer used for the DNA relaxation of DNA gyrase (Pierrat & Maxwell, 2003). Each 30 μL reaction contains 4-8 U of topoisomerase I from human (TopoGen) or wheat germ (Promega), 0.6 μg of negatively supercoiled pBR322 DNA in relaxation buffer [35 mM Tris.HCl pH 7.5, 24 mM KCl, 5 mM MgCl₂, 5 mM DTT, 6.5% glycerol (w/v), 0.36 mg/ml BSA, 9 μg/mL tRNA], and either 1% (Peptide 1) or 3.3% (EtBr, MccB17, CFX) DMSO. In one set of reactions, negatively supercoiled pBR322 DNA was relaxed at 37° C. for 30 minutes, then intercalative agent was added, i.e., the substrate of the assay was relaxed. In a second set of reactions, the intercalative agent was incubated with the DNA in reaction buffer at 37° C. for 15 minutes prior to the addition of topoisomerase I, i.e., the substrate of the assay was negatively supercoiled. These two different orders of addition of the reagents allows the distinction between DNA intercalation and inhibition of DNA relaxation by topoisomerase I. Concentrations of test compounds were varied as follows: 1, 10, and 50 μM MccB17 (lanes 2-5) or CFX (lanes 10-13); 0.5, 2, and 5 μM EtBr (lanes 6-9); 20, 50 and 100 μM Peptide 1 (lanes 14-19). Samples were incubated for 60 minutes at 37° C., followed by treatment with 1% SDS and 0.3 mg/mL proteinase K at 37° C. for 30 minutes. Reactions were terminated and samples were prepared for agarose gel electrophoresis as previously described (Heddle et al., 2001). Each reaction product was separated on 1% agarose gel, followed by staining in 1 μg/mL ethidium bromide.

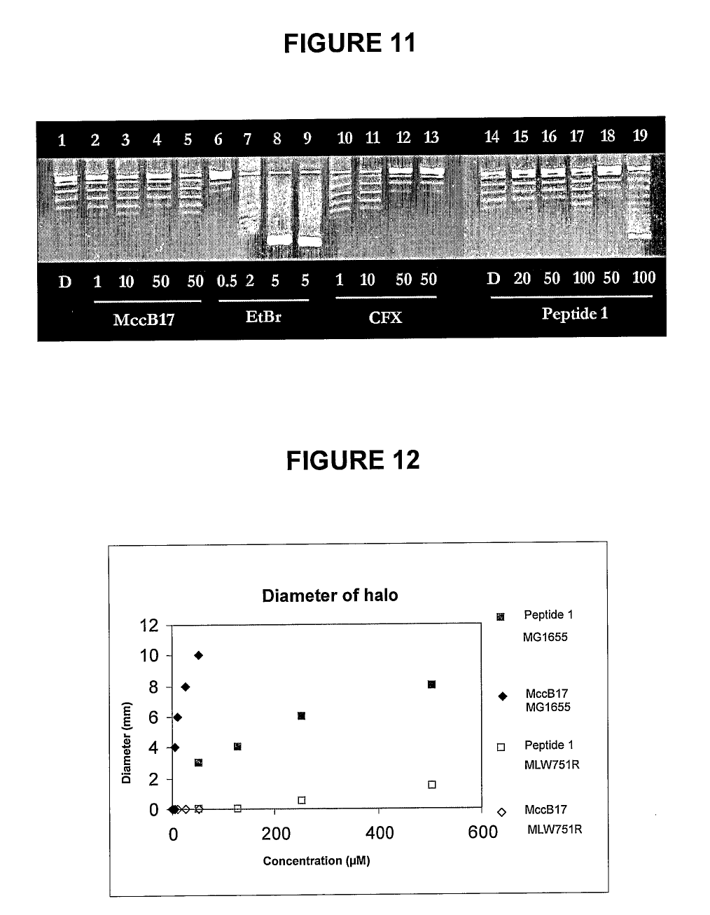

Result: Ethidium bromide was about a 100-fold more efficient intercalative agent than CFX: CFX produced only a weak intercalative property at the highest concentration tested of 50 μM (lanes 12 & 13) while EtBr could show a similar effect at the lowest concentration tested of 0.5 μM (lane 6). Neither Peptide 1 nor MccB17 could show any intercalative property like EtBr or CFX at the concentrations tested in the assay. However, Peptide 1 was also a weak inhibitor of the topoisomerase I relaxation reaction when tested at 100 μM with negatively supercoiled DNA as substrate of the reaction (lane 19). One cannot exclude the possibility that the absence of intercalation observed with 100 μM Peptide 1 (lane 18) was in fact due to inhibition of the topoisomerase I reaction. Further experiments to ascertain that, Peptide 1, like MccB17, is not an intercalating agent, are under way, see FIG. 11.

### Halo Assays

Lawns of cells were prepared by spreading, on top of a LB+Agar plate, 3 ml of melted LSS medium (half-agar strength: 0.3 g Agar/50 ml LB) inoculated with 100 μl of a 10 ml LB overnight culture of E. coli cells grown at 37° C. Halo assays with MccB17 generally used a DH5α strain (Novagen) but the inventors used the standard E. coli strain (K12) because it is from this strain that the microcin B17-resistant mutant W751R was obtained. Two strains were tested: the wild-type MccB17 sensitive E. coli MG1655 and the MccB17-resistant mutant MLW751R (Trp⁷⁵¹→Arg mutation in the DNA gyrase B subunit). 4 μl of MccB17 at various concentrations in 10% DMSO and 4 μL of Peptide 1 at various concentrations in 10% Me₂SO (Table 1) were spotted on the lawns. The plates were incubated at 37° C. overnight and growth inhibition was qualitatively analysed by measuring the diameter of each halo. The mutant bacteria are resistant to MccB17 and Peptide 1. The mutation is at a single point (W751R DNA gyrase B subunit) suggesting that both Peptide 1 and MccB17 have a common binding site on DNA gyrase. See Table 2 and FIG. 12.

TABLE 2

| | Average Diameter (mm) | | | |
|---|---|---|---|---|
| | MG1655 WT | | MLW751R Mutant | |
| concentration (μM) | MccB17 | Peptide 1 | MccB17 | Peptide 1 |
| 0 | 0 | | 0 | |
| 1 | 0 | | 0 | |
| 2.5 | 0 | | 0 | |
| 5 | 4 | | 0 | |
| 10 | 6 | | 0 | |
| 25 | 8 | | 0 | |
| 50 | 10 | 3 | 0 | 0 |
| 127 | | 4 | | 0 |
| 254 | | 6 | | 0.5 |
| 508 | | 8 | | 1.5 |

### Peptide 2

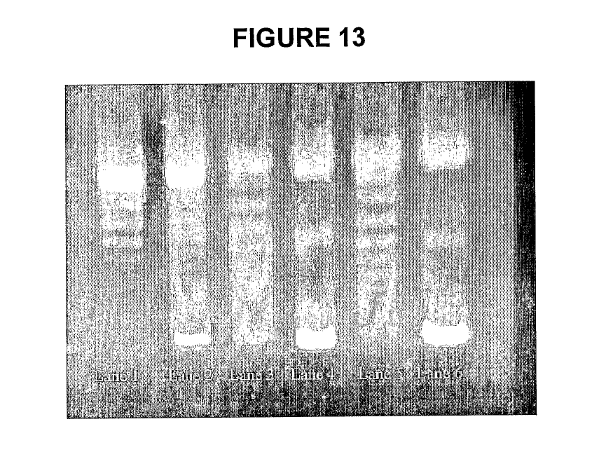

Structure: Peptide Ala-Gly-gly-X₂-Gly-Ala-Gln, where X₂ represents the 4-methylamino-1H-pyrrole-2-carboxylic acid, was synthetised using the solid phase method synthesis described above and tested in vitro, following the supercoiling assay method also described above. The assay results shown in FIG. 13 and indicate that Peptide 2 is about half as active as Peptide 1, as can be seen in the figure: Supercoiling assay Peptide 2: Lane 1: without A₂B₂, Lane 2: DMSO 10%, Lane 3: Peptide 1 (100 μM), Lane 4: Peptide 2 (100 μM), Lane 5: Peptide 2 (200 μM) and lane 6: Peptide 2 (50 μM).

### Example 5

### Relaxation Assays

Relaxation assays were performed in a manner analogous to that described by Pierrat et al., 2005. Briefly, relaxation assays were performed as for the supercoiling assay but with some modifications: ATP and spermidine were omitted. Gyr A and Gyr B were added to a solutions containing 35 mM of Tris.HCl (pH=7.5), 24 mM of KCl, 4 mM of MgCl₂, 1.8 mg/mL of spermidine, 6.5% of glycerol, 0.36 mg/mL of BSA, 9 μg/mL of tRNA, 5 mM of DTT, 2 mM of ATP and 24 nM of relaxed pBR322 DNA. The reaction contains A₂B₂ dimer and also Microcin B17 at 25 μM or hydrolysed Microcin B17 at various concentrations. The amount of DMSO was kept constant at 3.33%. The reactions were incubated at 25° C., and at each time point 30 μL aliquots were quenched with the addition of an equal volume of chloroform/isoamyl alcohol (24:1) and a half volume of loading buffer STEB (40% sucrose/100 mM Tris HCl, pH 7.5/100 mM EDTA/2 mg/mL bromophenol blue). The mixtures then were vortexed and centrifuged for 1 minute at 13,000 rpm. The aqueous phase was loaded onto 1% agarose, TAE (40 mM Tris-acetate, 1 mM EDTA) gels and were run at either 30 V overnight in TAE (in the cold room) or 70 V for 2.5 hours in TAE. The gels were stained for about 20 minutes in TAE containing 1 μg/mL ethidium bromide followed by destaining in multiple washes of TAE. The data were analysed using Syngel software. Concentration of A₂B₂ is 70 nM, concentrations of Peptide 1 and Peptide 2 are 25, 40, 50, 100 and 200 μM and concentration of peptide 5 are 10, 15, 20, 40 and 70 μM.

The relaxation assay gels for peptides 1, 2, and 5 are shown in FIG. 16, in which: (a) (Upper left side) Lane 1: no enzyme, Lane 2: enzyme+DMSO (10%), Lane 3: MccB17 at 25 μM, Lane 4, 5, 6, and 7: Peptide 1 at 25, 40, 50, 100 and 200 μM; (b) (Upper right side) Lane 1: no enzyme, Lane 2: enzyme+DMSO (10%), Lane 3: Peptide 1 at 100 mM, Lane 4, 5, 6, 7 and 8: Peptide 2 at 25, 40, 50, 100 and 200 μM; (c) (Lower side) Lane 1: no enzyme, Lane 2: enzyme+DMSO (10%), Lane 3, 4, 5, 6 and 7: Peptide 5 at 10, 15, 20, 40 and 70 μM, Lane 8: MccB17 at 25 μM.

Peptides 1, 2 and 5 inhibit the relaxation reaction at high concentration: 100 μM for Peptide 1; 200 μM for Peptide 2; 40 μM for Peptide 5. At such high enzyme concentration, MccB17 (Lane 3 in FIG. 16(a) and FIG. 16(b), Lane 8 in FIG. 16(c)) does not show any inhibition. Peptides 1, 2 and 5 seem to have the same inhibitory activity towards both the supercoiling and relaxation reactions.

### ATPase Assays

ATP hydrolysis by DNA gyrase was linked to the oxidation of NADH using a pyruvate Kinase (PK)/lactate dehydrogenase (LDH) coupled enzyme assay and measured at 340 nm on a Spectramax Plus Microplate. The ATPase assay was similar to that described previously by Pierrat & Maxwell, 2005. Each 100 μL reaction contained 50 mM Tris.HCl (pH 7.5), 24 mM KCl, 5 mM MgCl₂, 6.5% (w/w) glycerol, 4 mM dithiotreitol, 0.4 mM NADH, 0.8 mM phosphorenol-pyruvate, 1% (w/w) PK/LDH mixture (Sigma), 51 nM-153 nM gyrase enzyme and the drug tested at varying concentrations. DMSO was kept constant at 2%. The reactions were measured in the presence or absence of linear DNA pBR322 at fixed concentration of 8.4 nM. Reactions were initiated by the addition of 10 mM Mg.ATP and measured at 25° C. over 1.5 hours.

In DNA-independent ATPase reaction, formal concentration of subunit B₂ enzyme is 150 nM, concentrations of Peptide 1 are 34 and 103 μM and concentrations of Peptide 5 are 15 and 70 μM.

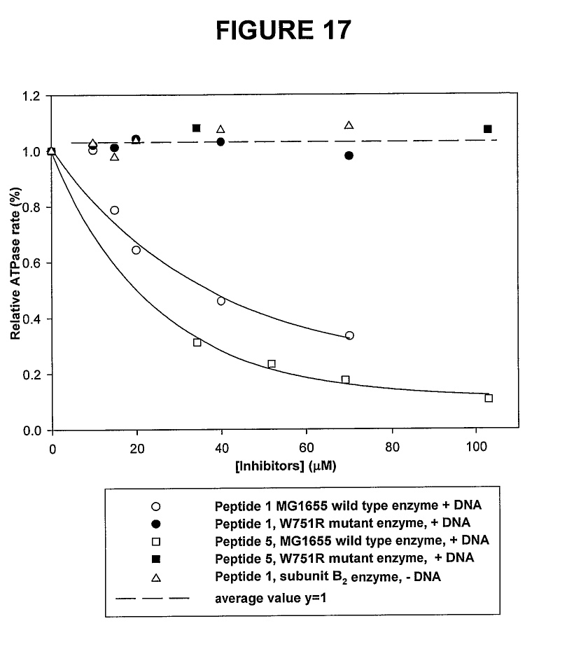

FIG. 17 is a graph of relative ATPase rate (%) versus concentration of inhibitor (μM) for Peptides 1 and 5, for: (a) Peptide 1, MG1655 wild type enzyme+DNA (open circles), (b) Peptide 1, W751R mutant enzyme+DNA (filled circles), (c) Peptide 5, MG1655 wild type enzyme+DNA (open squares), (d) Peptide 5, W751R mutant enzyme+DNA (filled squares), (e) Peptide 1, subunit B2 enzyme−DNA (open triangles), and (f) average value y=1 (dashed line).

As shown in the figure, Peptide 1 does not inhibit the DNA-independent ATPase reaction of the B₂ enzyme. The results obtained with Peptide 5 are similar (data not shown).

These results strongly suggest that:
(a) Peptide 1 and Peptide 5 do not bind in the N-terminal domain of the B subunit of DNA gyrase. This result is in agreement with the results obtained in the supercoiling assay. Indeed, the single point mutation W751R, located in the C-terminal of the B subunit, confers resistance to Peptide 1.
(b) Peptide 1 and 5, as well as MccB17, require DNA and the full-length A₂B₂ enzyme to inhibit the ATPase reactions.

DNA-dependent ATPase reactions were tested with the wild type enzyme A₂B₂ and the W751R mutant enzyme. In these DNA-dependent ATPase assays, the concentration of wild type A₂B₂ is 51 nM, the concentration of W751R mutant enzyme is 106 nM. Concentrations of Peptide 1 are 34, 52, 69 and 103 and concentrations of Peptide 5 are 10, 20, 30, 40 and 70 μM.

Experimental data were fitted and treated with the following equation:

$$\frac{v}{{v}_{0}} = {y}_{o} + a {e}^{( - bL )} \\ a \text{:} amplitude \\ b \text{:} first order rate constant \\ {y}_{0} \text{:} rate insensitive to L \qquad ( Equation 1 )$$

where L represents the inhibitor concentration, v represents the rate of the ATPase reaction, and v₀ represents the rate in absence of inhibitor. The IC₅₀ is deduced from the equation 2:

$$if {y}_{0} > a {IC}_{50} = \frac{1}{b} Ln ( \frac{1}{2} ( \frac{{y}_{0}}{a} - 1 ) ) \\ if {y}_{0} < a {IC}_{50} = \frac{- 1}{b} Ln ( \frac{1}{2} ( 1 - \frac{{y}_{0}}{a} ) ) \\ where \text{:} \\ y = \frac{( a + {y}_{0} )}{2} . \qquad ( Equation 2 )$$

The values of yₒ, a and b are summarized in the following Table:

TABLE 3
Fitted parameters (for Equation 1)

| | yₒ | a | b |
|---|---|---|---|
| Peptide 1 | 0.21 ± 0.01 | 0.81 ± 0.06 | 0.027 ± 0.003 |
| Peptide 5 | 0.11 ± 0.02 | 0.89 ± 0.03 | 0.041 ± 0.004 |

From Equation 2, the IC₅₀ values are, respectively, 37 μM and 15 μM for Peptide 1 and Peptide 5. Standard deviations are all less than 10%, confirming that the Equation 2 is a good model to describe the kinetic of the ATPase inhibition of both Peptide 1 and Peptide 5. Little or no inhibition was seen when increasing concentrations of Peptides 1 and 5 were tested with the W751R mutant A₂B₂ enzymes. This result supports the idea that the inhibition observed with the wild type enzyme is significant. The use of novobiocin and Ciprofloxacin at saturation, as inhibitors, eliminates the inactivity of both wild type and mutant enzyme. Novobiocin is a well-known competitive inhibitor of the ATPase reaction and the W751R mutation does not confer resistance to novobiocin.

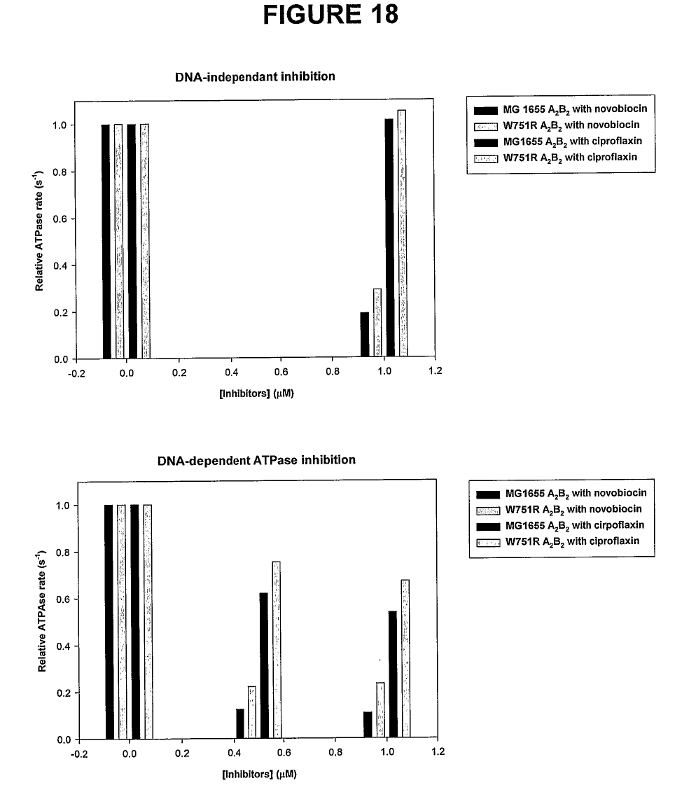

FIG. 18 shows the DNA-independent inhibition and DNA-dependent inhibition data (in terms of relative ATPase rate (s−1) versus concentration of inhibitor (μM)). In each cluster (at 0, 0.5, and 1.0 μM), the four peaks are, from left to right, (i) MG 1655 A2B2 with novobiocin, (ii) W751R A2B2 with novobiocin, (iii) MG1655 A2B2 with Ciprofloxacin, and (iv) W751R A2B2 with Ciprofloxacin.

As shown in the Figure, novobiocin inhibits both DNA-dependent and DNA-independent ATPase activity of wild type and W751R mutant enzyme. Ciprofloxacin does not inhibit the DNA-independent ATPase reaction of both the wild-type and mutant enzyme. The W751R mutation does not confer resistance to Ciprofloxacin, but as shown in FIG. 18, Ciprofloxacin slow down this ATPase hydrolysis rate of both wild type and mutant enzyme.

Biological activity data for Peptides 1, 2, 3, 4, and 5 are summarised in the following Table.

TABLE 4
In Vitro Biological Data

| | Supercoiling | | | | | |
|---|---|---|---|---|---|---|
| | (IC₅₀ μM) | | | | | ATPase |
| | MG 1655 | W751R | Cleavage | | Relaxation | % inhibition |
| Compound | A₂B₂ | A₂B₂ | (IC₅₀ μM) | Unwinding | (IC₅₀ μM) | at saturation |
| MccB17 | 3-fold | | 25 | Not an | 3-fold slow | 40 |
| | slow | | | intercalative | down | |
| | down | | | agent | | |
| Peptide 1 | 68 | >200 | inactive | Not an | 68 | 15 |
| | | | | intercalative | | |
| | | | | agent | | |
| Peptide 2 | >100 | — | inactive | — | >100 | — |
| Peptide 3 | inactive | — | inactive | — | — | — |
| Peptide 4 | Slow | — | inactive | — | — | — |
| | down | | | | | |
| Peptide 5 | 15 | — | inactive | — | 15 | 40 |

### Example 6

### Haloassay

Haloassays were performed as described above. Concentrations of Peptide 1 are 50, 125, 254 and 508 μM, concentrations of MccB17 are 50, 25, 10, 5 and 2.5 μM. According to the results obtained in vitro, Peptide 1 was tested against E. coli from DH5α strain to detect in vivo activity, and against E. coli from MG1655 strain and W751R strains to see if the single point mutation confers in vivo resistance to Peptide 1.

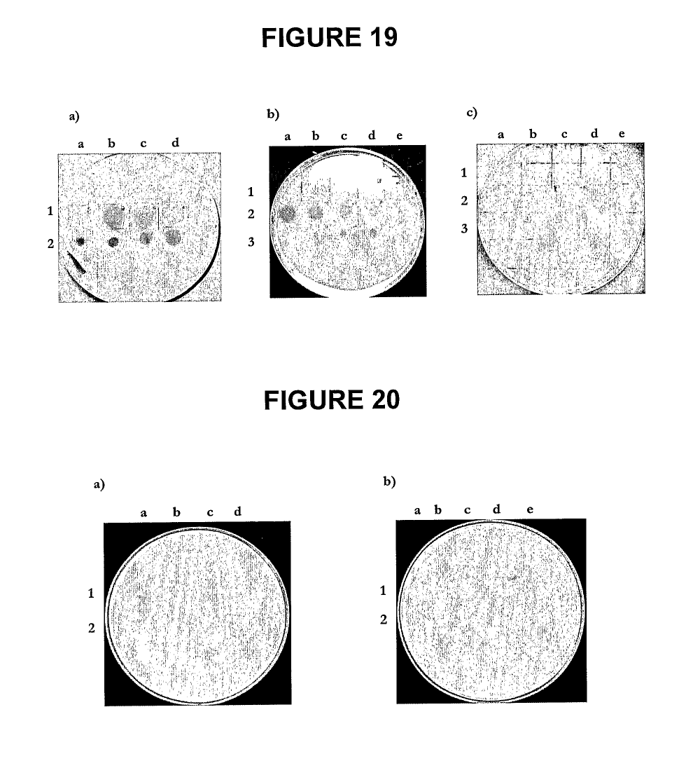

FIG. 19 provides a demonstration of bacterial growth inhibition for Peptide 1, in which:
(a) (Left side) E. coli DH5α 1a: DMSO (10%), 1b, 1c and 1d: Mccb17 at 150, 100 and 50 μM, 2a, 2b, 2c and 2d: Peptide 1 at respectively 125, 254, 508 and 1716 μM.
(b) (Middle) E. coli MG1655 1d: DMSO (10%), 1e: Mccb17 at 1 μM, 2a, 2b, 2c, 2d, and 2e: Microcin B17 at respectively 50, 25, 10, 5 and 2.5 μM, 3a, 3b, 3c and 3d: Peptide 1 at respectively 50, 125, 254 and 508 μM.
(c) (Right side) E. coli W751R: 1a: DMSO (10%), 2a, 2b, 2c, 2d and 2e: MccB17 at respectively 1, 2.5, 5, 10, 25 and 50 μM, 3a, 3b, 3c, and 3d: Peptide 1 at respectively 50, 125, 254, and 508 μM.

As shown in the figure, Peptide 1 is also active in vivo and the W751R mutation confers resistance to Peptide 1. At saturation of Microcin B17 (FIG. 19(a)), it can be seen that inside the no-growth area, some bacteria have still managed to develop (white spot). These MccB17 resistant bacteria have developed a mutation which targets the import system used by MccB17 to penetrate inside the cell. Surprisingly, these import mutant bacteria do not seem to be resistant to Peptide 1, suggesting that the import mechanism of Peptide 1 is different form the one used by MccB17. To ascertain this, import mutants bacteria are growth overnight and tested against MccB17 and Peptide 1.

FIG. 20 provides a demonstration of killing activity of Peptide 1 against E. coli DH5α import mutant, in which:
(a) (Right side) E. coli DH5α import mutant: 1a, 1b, 1c, 1d and: MccB17 at 25, 50, 100 and 150 μM, 2a: MccB17 at 10 μM, 2b, 2c and 2d: Peptide 1 at 58, 170, and 450 μM, 3c and 3d: Peptide 1 at 859 and 1000 μM.
(b) (Left side) E. coli DH5α: 1a, 1b, 1c, 1d and 1e: MccB17 at 10, 25, 50, 100 and 150 μM, 2a, 2b, 2c, 2d and 2e: Peptide 1 at 58, 170, 450, 859 and 1000 μM.

The absence of killing activity of MccB17 confirms that the bacteria are the import mutant ones.

Peptide 2 was only tested against the E. coli MG1655 strain. Concentrations of Peptide 2 are 100, 200 and 400 μM whereas concentrations of Peptide 1 are 50, 100 and 200 μM. The diameters of the killing zone are plotted against the concentration of the inhibitors to compare their potency.

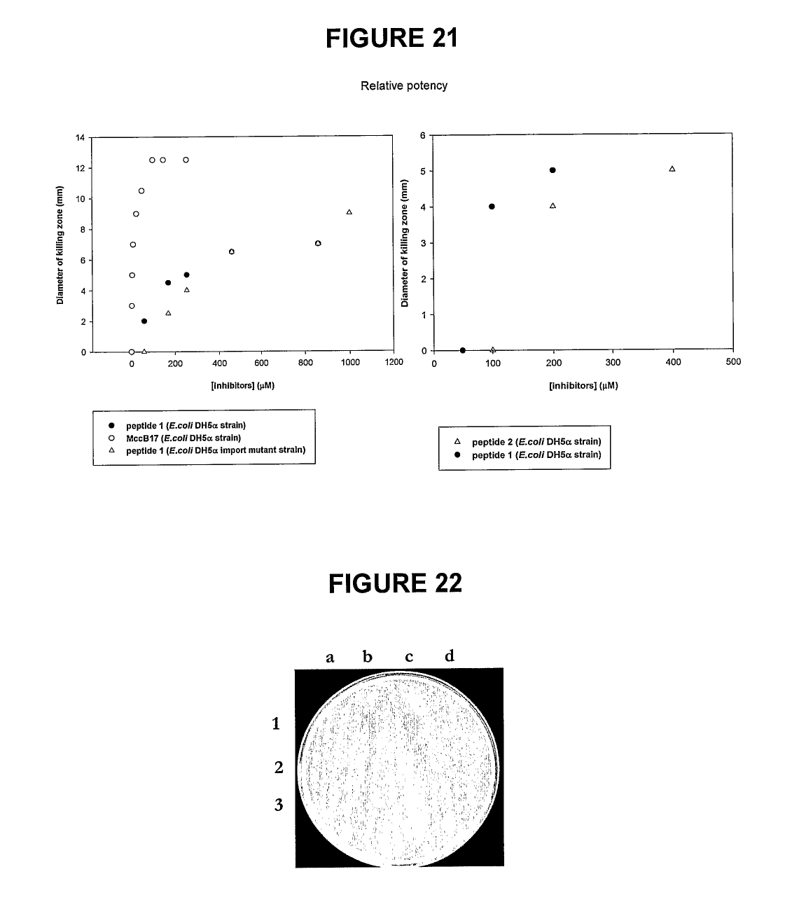

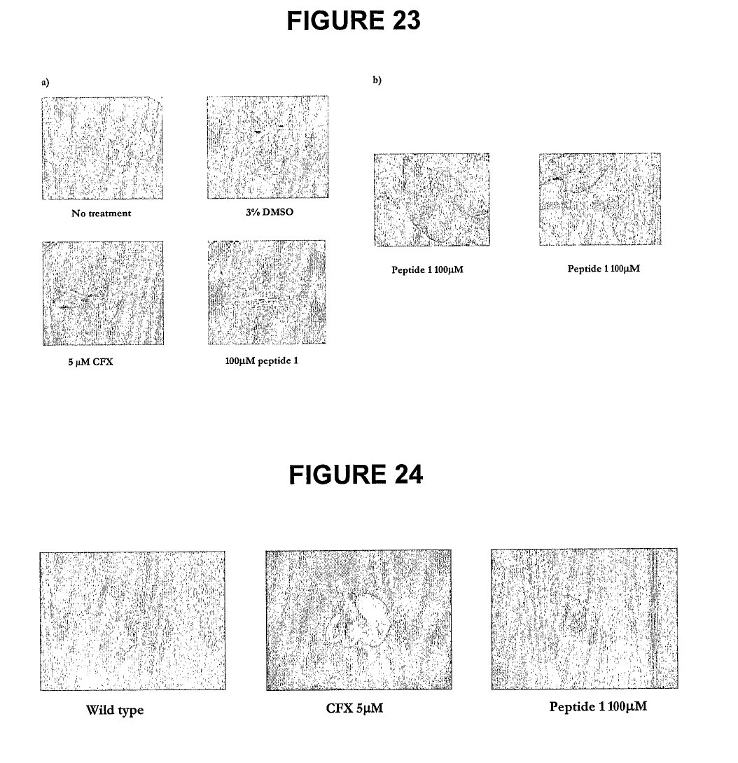

FIG. 21 shows graphically the relative potency, in terms of diameter of killing zone (mm) versus concentration of inhibitor (μM)).

Peptide 1 is 20 times less potent than MccB17 and does not use the same import mechanism as MccB17 to penetrate into the bacteria. Peptide 2 is twice less active than Peptide 1. This result is in agreement with the result obtained in vitro. Concentrations of Peptide 5 are 20, 150, 300, 500 and 1500 μM.

FIG. 22 shows the haloassay for Peptide 5, in which: 1a: DMSO (10%), 1b and 1c: MccB17 at 25 and 50 μM, 2a, 2b, 2c, 2d and 3d: Peptide 5 at 1500, 500, 300, 150 and 20 μM.

### Example 7

### Seedling Experiments

In parallel, the germination of A. thaliana ecotype Columbia seeds was totally inhibited, as shown in FIG. 24. Germination begins when the dormant dry seed begins to take up water (imbibition from the surface sterilization process). Primary roots emerge while the seed is in the culture media. Subsequently the hypocotyls emerge and elongate to pull the cotyledons above the culture media surface. After straightening up, the cotyledons arrange into an horizontal position, as shown in the Figure, and, then spread apart in order to expose the first true leaves and the apical meristem (growing tip). In the presence of CFX, the growth is inhibited immediately after germination, on the uptake of the CFX by the root. In the presence of Peptide 1, germination is totally inhibited (even the primary root, the radical, does not emerge), suggesting that Peptide 1 is a stronger inhibitor than CFX and manages to penetrate into the germinating tissue immediately after the opening of the seed coat.

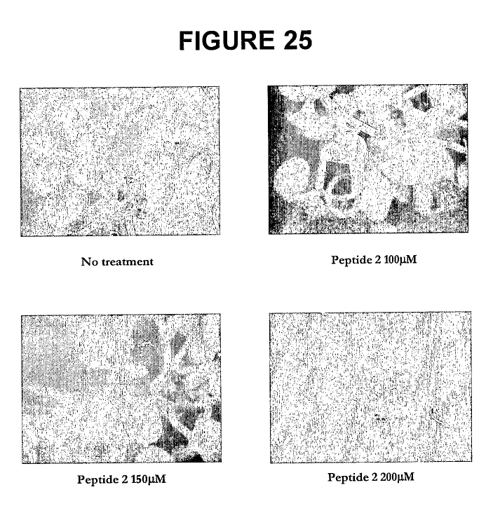

FIG. 25 shows photographs of the germination of A. thaliana ecotype Columbia (36 hours) for: no treatment (top left), 100 μM Peptide 2 (top right), 150 μM Peptide 2 (bottom left), and 200 μM Peptide 2 (bottom right).

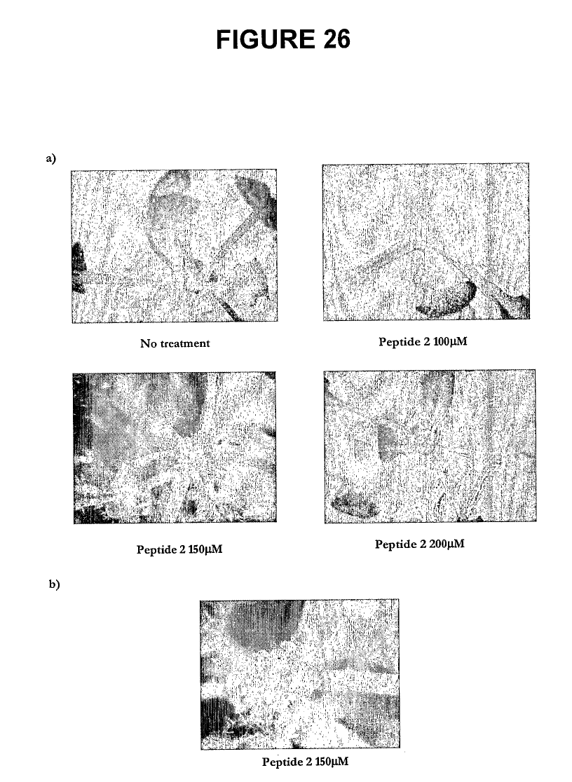

FIG. 26 shows photographs of effects of Peptide 2 on 6-week old A. thaliana ecotype Columbia seedlings, for (a) no treatment (top left), 100 μM Peptide 2 (top right), 150 μM Peptide 2 (bottom left), and 200 μM Peptide 2 (bottom right), and (b) tumour-like growth observed at 150 μM Peptide 2.

At 200 μM, the effects of Peptide 2 on both 6-week old seedlings and germinating seeds are the same as the effects observed for 100 μM Peptide 1. At 150 μM, seed germination occurred but the growth is slower compared with the untreated seeds aind bleaching of the emerging leaves is observed. At 100 μM, only a bleaching effect ori the emerging leaves is observed. The effect of Peptide 2 on 6-week old seedlings is very unusual: it induces tumour-like growth near the apical meristem zone.

Peptide 3 did not demonstrate activity against either seed germination or 6-week old seedlings.

The results of the plant assays are in agreement with the results obtained against bacteria. They show that both Peptides 1 and 2 are active and that Peptide 2 has half the potency of Peptide 1.

### Example 8

### Further Characterization of the Activities of Microcin B17-Based Heterocyclic Compounds Against DNA Topoisomerases

The inventors have demonstrated that several of the synthesized heterocyclic compounds, including Peptides 1, 2, and 5 inhibit E. coli DNA gyrase, at micromolar concentrations, and that the solubility of MccB17 is about 60 μM in water containing 5% DMSO, while Peptides 1, 2 and 3 are at least 20 times more soluble than the MccB17. Further, Peptides 1, 2 and 5 have similar inhibitory activity towards both the supercoiling and relaxation reactions catalysed by gyrase. Neither Peptide 1 nor Peptide 2 stabilise the DNA gyrase cleavage complex. Peptide 1 and Peptide 5 do not bind to the N-terminal domain of the B subunit of DNA gyrase. Peptide 1 and Peptide 5, as well as MccB17, require the full-length A₂B₂ enzyme and DNA to inhibit the ATPase reaction. Peptide 1 is less potent than MccB17 in halo assays and does not appear to use the same import mechanism as MccB17 to penetrate into E. coli. At a molecular level, Peptide 1 and MccB17 may have an overlapping binding site, because the W751R single point mutation which confers resistance to MccB17 also confers resistance to Peptide 1. E. coli MccB17 resistant import mutants are not resistant to Peptide 1.

DNA gyrase A protein (GyrA) and DNA gyrase B protein (GyrB), E. coli Topoisomerase IV, supercoiled and relaxed plasmid pBR322 DNA substrates and kinetoplast DNA were all purchased from John Innes Enterprises (Norwich, UK). Wheatgerm topoisomerase I was obtained from Promega (Madison, Wis., USA). Human topoisomerase I and topoisomerase IIα were purchased from TopoGEN Inc. (Port Orange, Fla., USA). Microcin B17 was a gift from Dr O. A. Pierrat (John Innes Centre, Norfold, UK). Camptothecin, m-AMSA and general reagents were purchased from Sigma (Gillingham, UK).

DNA gyrase mediated supercoiling assays were performed as previously described (see Reece and Maxwell, 1989). Gyrase (0.4 nM) was added to reactions containing 35 mM Tris.HCl pH 7.5, 24 mM KCl, 4 mM MgCl₂, 6.5% glycerol, 0.36 mg/mL BSA, 9 μg/mL tRNA, 5 mM DTT, 2 mM ATP, 4.6 nM of relaxed pBR322 DNA, and inhibitor where appropriate.

Topoisomerase IV-mediated relaxation assays and decatenation assays were performed as previously described (see Peng and Marians, 1993). Relaxation assays contained topoisomerase IV (20 nM), 40 mM Tris.HCl pH 7.5 at 30° C., 6 mM MgCl₂, 10 mM DTT, 1 mM spermidine.HCl, 20 mM KCl, 1 mM ATP, 0.5 mg/mL BSA, 4.6 nM of supercoiled pBR322 DNA and inhibitor where appropriate. Decatenation assays contained topoisomerase IV (4 nM), 40 mM Tris HCl pH 7.5 at 30° C., 6 mM MgCl₂, 10 mM DTT, 1 mM spermidine.HCl, 100 mM potassium glutamate, 0.5 mM ATP, 0.5 mg/mL BSA, 200 ng of kinetoplast DNA, and inhibitor where appropriate.

Wheatgerm topoisomerase I-mediated relaxation assays were performed as per the manufacturer's instructions. Topoisomerase I (2 nM) was added to reactions containing 50 mM Tris. HCl pH 7.5, 50 mM NaCl, 0.1 mM EDTA, 1 mM DTT, 20% glycerol, 4.6 nM of relaxed pBR322 DNA, and inhibitor where appropriate.

Human topoisomerase I-mediated relaxation assays were performed as per the manufacturer's instructions. Topoisomerase I (2 nM) was added to reactions containing 10 mM of Tris.HCl, pH 7.9, 150 mM NaCl, 100 μM spermidine.HCl, 5% glycerol, 0.1% BSA, 4.6 nM of relaxed pBR322 DNA, and inhibitor where appropriate.

Human Topoisomerase IIα-mediated relaxation assays and decatenation assays were performed as per the manufacturer's instructions in a total of 20 μL. Relaxation assays contained Topoisomerase IIα (4.3 nM), 50 mM Tris.HCl pH 8,120 mM KCl, 10 mM MgCl₂, 0.5 mM DTT, 0.5 mM ATP, 4.6 nM of supercoiled pBR322 DNA, and inhibitor where appropriate. Decatenation assays substituted the supercoiled DNA with 200 ng catenated kinetoplast DNA.

All in vitro reactions were incubated at 37° C. for 30 minutes. The DMSO concentration in each reaction was kept constant at 3.33%. Reactions were quenched with the addition of an equal volume of phenol/chloroform/isoamyl alcohol (25:24:1) and one tenth volume of loading buffer containing 40% sucrose, 100 mM Tris.HCl pH 7.5, 100 mM EDTA, 2 mg/mL bromophenol blue. The proteins were precipitated by vortexing, followed by centrifugation for 5 minutes at 16 000 g. The aqueous phase was loaded onto 1% agarose gels in Tris-acetate and were electrophoresed at 60 V for three hours in Tris-acetate buffer. The gels were stained in 1 μg/mL ethidium bromide. The gels were visualised using UV light and were documented and analysed using Syngene software.

### Results

### In Vitro Assays to Determine Activity of Boc Moieties

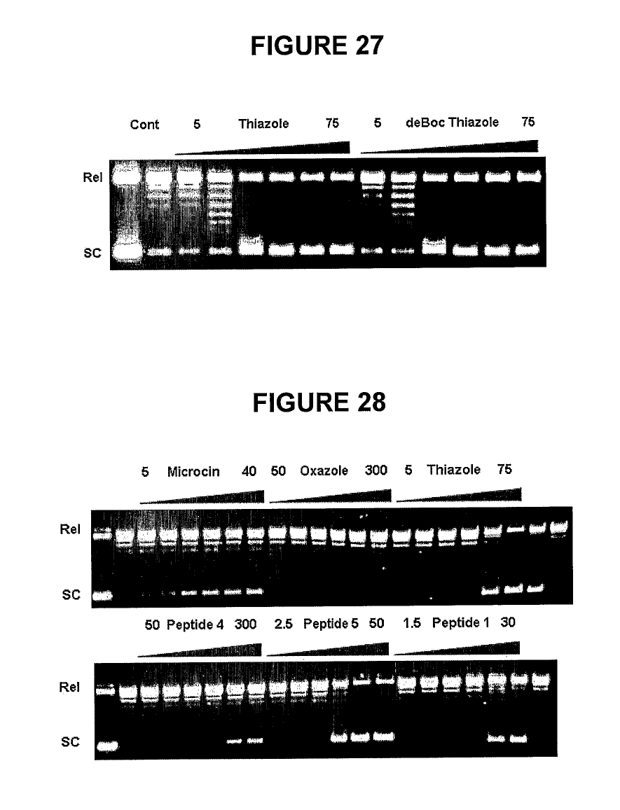

Synthesis of the heptapeptides followed a Boc solid phase strategy. During synthesis of the peptide moieties, stepwise extension of the immobilized intermediates preceded a final Boc-deprotection step to yield the immobilized heterocyclic compounds. The Boc residue had not been removed during preparation of the compounds. In order to confirm that the inhibition of the topoisomerases tested was due to the heterocyclic moiety, rather than the contaminating Boc group, the thiazole compound was further purified by acid cleavage and deprotection. The deBOC compound was then tested against its thiazole precursor in in vitro topoisomerase assays. For all compounds tested, the effects of the compounds were identical and there was no difference to the calculated IC₅₀ values of the thiazole and deBoc-thiazole compounds. Although only one de-protected compound was tested, it is reasonable to expect that it is the heterocyclic moiety that confers the inhibitory effects on topoisomerases, rather that the Boc group. See FIG. 27.

### In Vitro Experiments—Relaxation of Supercoiled DNA Catalysed by Human and Wheatgerm Topoisomerase I

It has been shown that MccB17 inhibits the relaxation activity of DNA gyrase. Several other topoisomerases which catalyse the relaxation of supercoiled DNA substrates were tested in in vitro assays in order to determine whether they are also inhibited by the MccB17-derivatives. Two sources of eukaryotic topoisomerase I were available: human (see FIG. 28) and wheatgerm (data not shown). Peptides 1, 5 and MccB17 inhibited both enzymes at similar concentrations. The thiazole heterocycle only inhibited human topoisomerase I. The oxazole based compounds were not active against the type I topoisomerases at the concentrations tested. The calculated IC₅₀ values for both enzymes are shown in the Table below.

### In Vitro Experiments—Relaxation of Supercoiled DNA Catalysed by Human Topoisomerase IIα

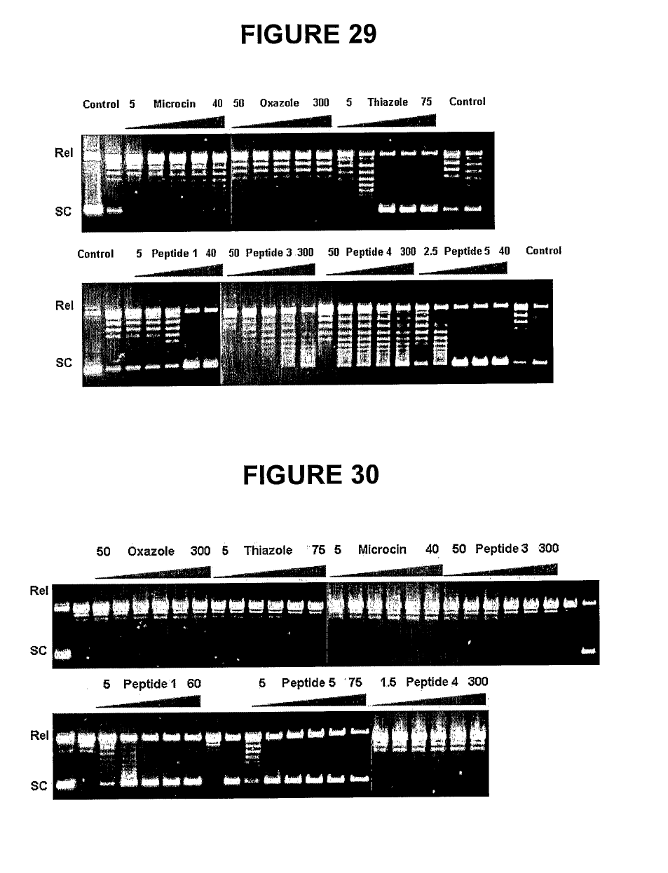

Human topoisomerase IIα catalyses the relaxation of DNA supercoils. These studies demonstrate that the essential replication enzyme is exquisitely sensitive to Peptide 1, Peptide 5, and the thiazole moiety, all with apparent IC₅₀'s below 20 μM. Surprisingly, Peptide 3 also exerted an effect on enzyme activity. Peptide 4 showed similar inhibitory activity with an IC₅₀ of 225 μM. The calculated IC₅₀ values for human topoisomerase IIα are shown in the Table below. See also FIG. 29.

### In Vitro Experiments—Relaxation Catalysed by E. Coli Topoisomerase IV

The two type II topoisomerases in E. coli, DNA gyrase and topoisomerase IV, share considerable amino acid sequence similarity, yet they have distinctive topoisomerization activities. These studies demonstrate that the DNA relaxation activity of topoisomerase IV is sensitive to Peptide 1 and Peptide 5 with apparent IC₅₀'s below 10 μM. See FIG. 30. The calculated IC₅₀ values are shown in the Table below. Despite the sequence similarity, topoisomerase IV appears to be more sensitive to the heterocyclic compounds than DNA gyrase. This may be due to a preference of the heterocyclic compounds for a specific conformation of the enzyme or residues that are more accessible in topoisomerase IV.

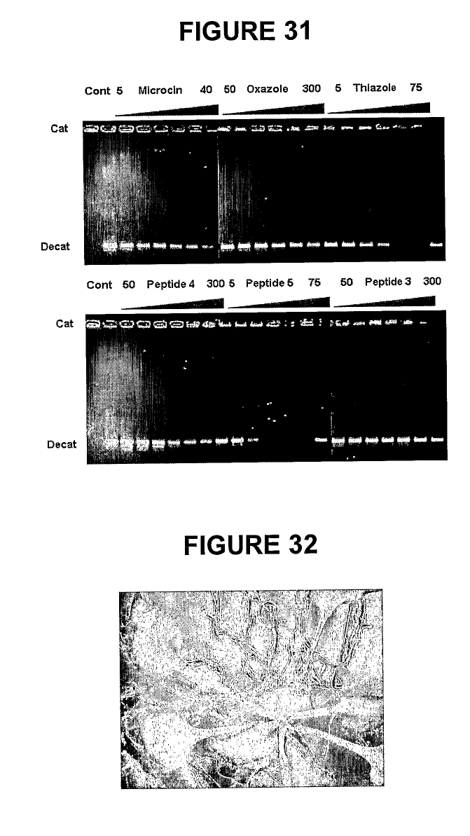

Topoisomerase IV and eukaryotic topoisomerase IIα share the ability to be able to both relax supercoiled DNA as well as catalyse their preferred reaction, the decatenation of knotted or interlinked circles of DNA. The decatenation activity of both topoisomerase IV (see FIG. 31) and eukaryotic topoisomerase IIα (data not shown) are exquisitely sensitive to Peptide 1, Peptide 5, and the thiazole moiety. Surprisingly, all compounds tested exerted an effect of enzyme activity, albeit weakly in the case of Peptide 3 and the oxazole moiety. The calculated IC₅₀ values are shown in the Table below.

In Vivo Experiments in Arabidopsis thaliana

The microcin derivatives were tested for efficacy in planta against Arabidopsis thaliana ecotype Columbia according to known methods (see Wall et al., 2004). Seeds were germinated on sterile media containing the appropriate peptide and the effects were observed over a three week period. Peptides 1 and 2 were able to inhibit germination of the seeds. The remaining heterocyclic compounds tested and the DMSO controls did not exert an effect on plant growth at the concentrations tested. It is probable that the peptide compound is rapidly taken up during the imbibition process, as the hypocotyls and root primordial did not emerge from the testa. These compounds are significantly more toxic to germinating seedlings than CFX, possibly because they inhibit multiple topoisomerases. In starch-storing seeds, such as A. thaliana, during imbibition the quiescent seed rapidly resumes metabolic and respiratory activity. One of the first steps, as part of this process, is the initiation of mitochondrial DNA synthesis which requires DNA gyrase and which may explain the exquisite sensitivity of the germinating seeds to Peptides 1 and 2.

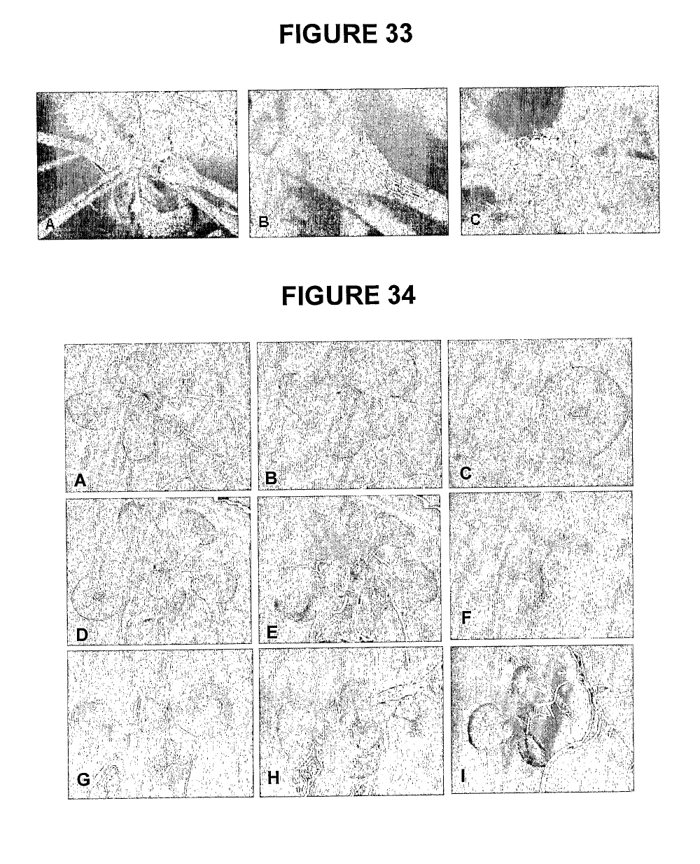

When 4-week old Arabidopsis plants were transferred to media containing 100 μM Peptide 1, the leaves rapidly wilted. The effects of the drug were exacerbated, as Peptide 1 then appeared also to be taken up by the wilted leaves on contact with the media. The leaf tissue initially turned yellow, followed by rapid necrosis of the tissue (see FIG. 32). Peptide 2 (200 μM) induced tumourous, undifferentiated cell growth in the meristematic region and petioles where active chloroplast and mitochondrial DNA replication takes place (see FIG. 33). The remaining heterocyclic compounds did not exert an effect on plant growth at the concentrations tested. The high hydrophility of Peptide 1 may preferentially allow the compound to readily diffuse through the leaf cuticle and epidermis to the mesophyll and phloem below.

The heterocyclic compounds were also spotted onto the leaves of 4 week old, tissue culture grown Arabidopsis thaliana ecotype Columbia plants (see FIG. 34, panels A-1). The effects of Peptide 1 (100 μM) are exceptionally rapid, with the first appearance of tissue browning occurring 5 to 10 minutes (see FIG. 34, panel C) after application of the compound on to the surface of the leaf. The browning and necrosis diffused through the full thickness of the leaf over the next 60 minutes (see FIG. 34, panel F) and spread towards the meristem (see FIG. 34, panels E, F, H). Complete necrosis of the meristem and newly emerged leaves, where organellar replication takes place, was complete within 24 hours (see FIG. 34, panel F). Application of 20 μM Peptide 1 led to localised browning and necrosis, but did not cause the systemic effects observed at higher concentrations of the compound.

It is probable that Peptide 1 is inducing the hypersensitive response (HR), a defense response normally elicited by plant pathogens. Cellular apoptosis, as part of the HR, can be observed approximately 4 hours after plants are treated with pure cultures of pathogenic bacteria or fungi (F. Jun, pers. comm.). It is likely that the exceptionally rapid response elicited by Peptide 1 in these studies is due to the purity of the compound as well as a high affinity for the HR signal recognition proteins.

In Vivo Experiments with Human Cell Cultures

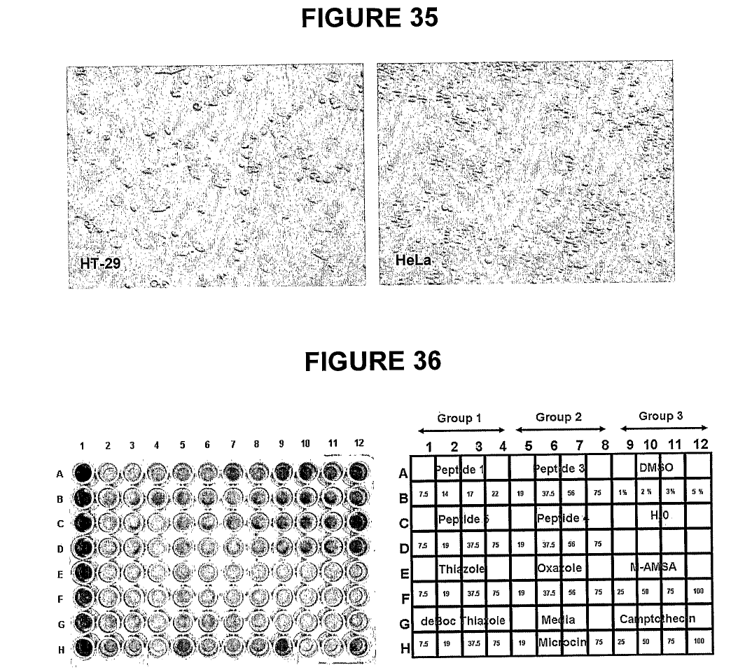

Human cell cultures were initiated from frozen cell stocks (the gift of Dr J. Gavrilovic, UEA). The HT-29 line is derived from a colorectal adenocarcinoma while HeLa cells are derived from a cervical epithelial adenocarcinoma (see FIG. 35). Both human cell lines form adherent cultures and were maintained at 37° C. in T25 culture vessels as 10 mL cultures in CO₂ Independent Media containing 10% foetal bovine serum (EU approved), 4 mM L-glutamine, penicillin/streptomycin (Invitrogen). A subcultivation ratio of 1:3 and 1:6 were maintained for HT-29 and HeLa lines respectively. Media was renewed every three days.

The assay to determine the effects of the heterocyclic compounds on mammalian cells is based on the cleavage of the yellow tetrazolium salt MTT to purple formazoan crystals by metabolically active cells (Roche). After incubation with MTT and solubilization, the resulting coloured solution is quantified using a scanning spectrophotometer. Each of the cell lines was trypsinised and resuspended in fresh media prior to transfer to sterile flat-bottomed microtitre plates (200 μL per well) and allowed to recover for 16 hours. Five thousand HT-29 or 7500 HeLa cells per 100 μL culture medium were incubated in the presence of the drugs in various concentrations (totalling 10 μL) for 24 hours. Cell viability was then determined by addition of MTT reagent (20 μL) in each well, as per manufacturer's instructions and incubated for a further 4 hours. Solubilization solution (100 μL) was added per well and the plates incubated overnight at 37° C. The plates were read in a spectrophotometer (scanning from 500-650 nm, signal peak at 570 nm). A representative result is illustrated in FIG. 36.

The cell-based assays yielded similar responses to the in vitro assays, with Peptides 1, 5, and the thiazole moieties (both protected and de-protected) inhibiting cell viability at concentrations consistent with inhibition of the purified enzymes. Approximate IC₅₀'s for the heterocyclic compounds against both human cell lines are shown in the Table below. The HeLa cells showed a slightly different response to the heterocyclic compounds tested.

The HT-29 line is highly resistant to current anti-cancer therapies and the results of these studies are very encouraging. Increased expression of topoisomerase I was observed in colorectal tumors compared with their normal tissue counterparts (Husain et al., 1994), enhancing the possibility of using a topoisomerase I inhibitor as a promising anticancer drug. Camptothecin (CPT) analogs, which target topoisomerase I, such as topotecan and irinotecan (CPT-11), are among the most effective anticancer drugs in use (see, e.g., Potmesil, 1994; Dancey and Eisenhauer, 1996).

G2 phase cell cycle arrest is a common cellular response to DNA damage and is also referred to as a checkpoint response to DNA damage (see Hartwell and Weinert, 1989). The G2/M checkpoint helps to prevent further damage and gives the cell time to repair the lesions that have already occurred. This serves to preserve viability and to maintain the integrity of the genome. Inhibition of both type I and type II topoisomerases by the heterocyclic compounds would have implications for all stages of the cell cycle.

### CONCLUSIONS

The thiazole-derived moieties are more active than the oxazole-derived compounds. The α,α′ linker orientation is one of the key factors for the activity of the peptide as for the analogues of MccB17. The synthesized heterocyclic oligopeptides demonstrate significantly higher activities. It is likely that the peptide moiety facilitates active transport across the cell membranes resulting in intracellular accumulation. Peptide 1 and Peptide 5 totally inhibit, at saturation, the supercoiling and relaxation reactions as well as the DNA-dependent ATP hydrolysis by DNA gyrase, whereas they apparently do not stimulate the formation of the gyrase-dependent cleavage-complex formation. Moreover Peptide 1 antagonizes quinolone cleavage complex. This result suggests that both Peptide 1 and Peptide 5 have a stronger binding affinity to DNA gyrase than MccB17. In these studies, Peptide 5 appears to be the most effective compound in vitro. The penetration capacity of Peptide 1 makes this the most effective compound tested in vivo. Each compound, including Peptide 3, has in vitro activity against at least one Topoisomerase. These studies begin to map a mode of action for Peptide 1 and Peptide 5, which have common features with the mode of action of MccB17 and simocyclinones. It has been shown than simocyclinone D8 inhibits the supercoiling and relaxation reactions catalysed by DNA gyrase but not the ATPase reaction (see Flatman et al., 2004). Moreover, simocyclinone D8 does not stimulate the gyrase-dependent cleavage complex formation but antagonizes quinolone cleavage. Peptide 1 and Peptide 5 totally inhibit the supercoiling and relaxation reactions as well as the DNA-dependent ATP hydrolysis by DNA gyrase, whereas they do not stimulate the formation of the gyrase-dependent cleavage complex formation. Moreover Peptide 1 antagonizes quinolone cleavage complex. At a molecular level, it seems that Peptide 1 and MccB17 have overlapping binding site, because the W751R single point mutation confers also resistance to Peptide 1. However, it is not possible from these studies to speculate exactly how any of the heterocyclic compounds interact with topoisomerases. Whatever the mechanism of interaction, they remain potent inhibitors of both classes of the essential replication enzymes. Increased efficacies of the compounds may be achieved by the selection of alternative oligopeptide carriers (see Diddens, 1976). While the majority of topoisomerase inhibitors show selectivity against either type I or type II topoisomerases, a small number of compounds can act against both classes of enzymes (Denny, 2003). The multimodal function of these heterocyclic compounds should prevent resistance developing readily as simultaneous mutations in both type I and type II topoisomerases is unlikely to occur in vivo. The two independent peptides can act synergistically, probably by attacking the topoisomerase target simultaneously in two different sites of interaction. The improved efficacy would therefore reduce the required therapeutic dose and provide the microcin-derived peptide analogues with acceptable, if not good, safety margins. The results of the mammalian cell cultures experiments confirm that these peptides are good candidates as anti-tumour agents.

TABLE 5-A
Biological Data

| | Names and | |
|---|---|---|
| # | Chemical Identity | Chemical Structure |
| 1 | Microcin | |
| 2 | Peptide 13.13APyrrole 2,5 sub′n | 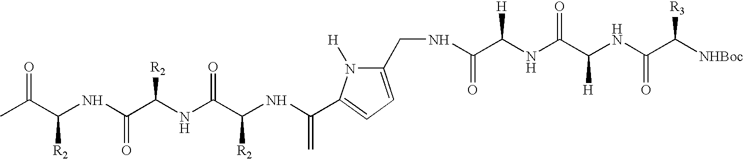 |
| 3 | Peptide 23.13BPyrrole 2,4 sub′n | 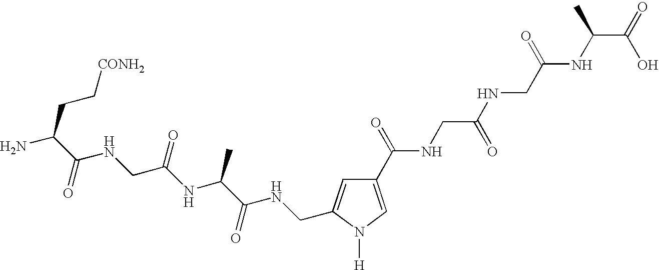 |
| 4 | Peptide 3Isoxazole | 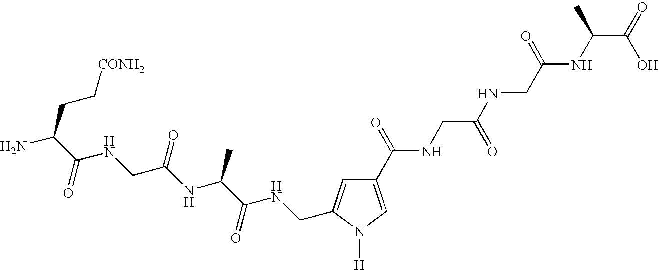 |
| 5 | A2.4Heterocycle thiazole | 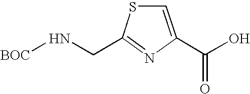 |
| 6 | B2.8Heterocycle oxazole | 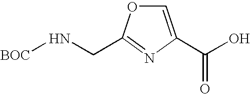 |
| 7 | C2.17Heterocycle Bis oxathiazole | 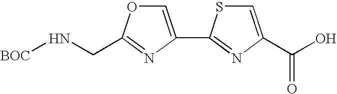 |
| 8 | D2.13Heterocycle Bis thiaoxazole | 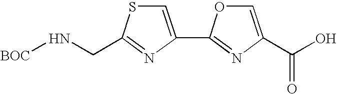 |
| 9 | A5Pep5Heptapeptidethiazole | 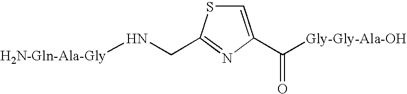 |
| 10 | B6Pep4Heptapeptideoxazole | 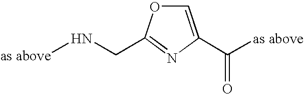 |
| 11 | C7HeptapeptideBis oxathiazole | 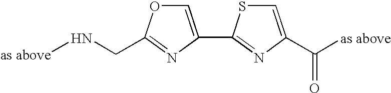 |
| 12 | D8HeptapeptideBis thiaoxazole | 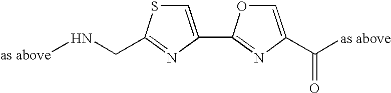 |

TABLE 5-B
Biological Data

| | | Topo IIα | Topo I | Topo I | |
|---|---|---|---|---|---|
| # | Topo IV | Human | Wheatgerm | Human | ATPase |
| 1 | R > 35 μM | R > 30 μM | 37 μM | 37 μM | Inhibits A₂B₂ + |
| | D 25 μM | D NA > 36 μM | | | DNA dep |
| 2 | R 10 μM | R 20 μM | 55 μM | ~35 μM | Inhibits, A₂B₂ + |
| | D <1 μM | D <1 μM | | | DNA dep |
| | | | | | IC50 ~50 μM |
| 3 | NT | NT | NT | NT | Inhibits, A₂B₂ + |
| | | | | | DNA dep |
| | | | | | IC₅₀ ~100 μM |
| 4 | R NA > 300 μM | R > 250 μM | NA > 300 μM | NA > 300 μM | Not Active |
| | D NA > 300 μM | D < 50 μM | | | |
| 5 | R >75 μM ? | R 15 μM | NA > 75 μM | 40 μM | Inhibits A₂B₂ + |
| | D 20 μM | D < 5 μM | | | DNA dep |
| 6 | R NA > 300 μM | R NA > 250 μM | NA > 300 μM | NA > 300 μM | |
| | D 200 μM | D NA > 250 μM | | | |
| 7 | NT | NT | NT | NT | NT |
| 8 | NT | NT | NT | NT | NT |
| 9 | R 7.5 μM | R 7 μM | 30 μM | 25 μM | Inhibits |
| | D 10 μM | D < 2.5 μM | | | A₂B₂ + DNA |
| | | | | | dep |
| 10 | R NA > 300 μM | R 225 μM | NA > 300 μM | NA > 300 μM | |
| | D 175 μM | D < 50 μM | | | |
| 11 | NT | NT | NT | NT | NT |
| 12 | NT | NT | NT | NT | NT |

TABLE 5-C
Biological Data

| | | | | | | In vivo | In vivo |
|---|---|---|---|---|---|---|---|
| | IC₅₀ SC | IC₅₀ | IC₅₀ | In vivo | In vivo | HT29 | HeLa |
| # | gyrase | relaxation | cleavage | E. coli | Plants | Cells | Cells |
| 1 | 30 μM | Inhibits | Stabilizn of | Yes | Not | Not | Not |
| | | IC50 ?? | cleavage | | Active | Active | Active |
| | | | cpx | | | >50 μM | >50 μM |
| | | | 25 μM | | | | |
| 2 | 70 μM | ~100 μM | No | Yes | S 20 μM | ~15 μM | ~25 μM |
| | | | stabilizn of | 20x < act | P 20 μM | | |
| | | | cleavage | cf | | | |
| | | | cpx | micro | | | |
| | | | | M/M | | | |
| 3 | 140 μM | 200 μM | No | Yes | S ≧ 200 μM | NT | NT |
| | 2x < act cf | | stabilizn of | | P ≧ 200 μM | | |
| | pep1 | | cleavage | | | | |
| | | | cpx | | | | |
| 4 | Not Active | Not | No | Not | Not | Not | Not |
| | | Active | stabilizn of | Active | Active | Active | Active |
| | | | cleavage | | >300 μM | >75 μM | >75 μM |
| | | | cpx | | | | |
| 5 | 75 μM | | | Not | Not | ~15 μM | ~75 μM |
| | | | | Active | Active | deBoc | deBoc |
| | | | | (→100 μM) | P > 75 μM | ~15 μM | ~75 μM |
| 6 | < act cf A | | | Not | Not | Not | Not |
| | | | | Active | Active | Active | Active |
| | | | | (→400 μM) | P > 300 μM | >75 μM | >75 μM |
| 7 | Inactive | | | NT | NT | NT | NT |
| 8 | Inactive | | | NT | NT | NT | NT |
| 9 | 25 μM | 40 μM | No | Not | Not | ~15 μM | ~75 μM |
| | | | stabilizn of | Active | Active | | |
| | | | cleavage | (→1500 μM) | P > 75 μM | | |
| | | | cpx | | | | |
| 10 | 400 μM | | No | Not | Not | Not | Not |
| | | | stabilizn of | Active | Active | Active | Active |
| | | | cleavage | | P > 300 μM | >75 μM | >75 μM |
| | | | cpx | | | | |
| 11 | | | NT | NT | NT | NT | NT |
| 12 | | | NT | NT | NT | NT | NT |

### Abbreviations:

Act: active
CFX: ciprofloxacin
CPX: complex
D: decatenation reaction
Dep: dependent
NA: not active
NT: not tested
P: whole plants (expanded Arabidopsis leaves)
R: relaxation reaction
S: seeds
SC: supercoiling reaction
Stabilizn: stabilization
Sub'n: substitution

### REFERENCES

A number of publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.
Andreu, D., Rivas, L., 1998, Biopolymers, Vol. 47, pp. 415-433.
Champoux, J. J., 2001, Annual Review in Biochemistry, Vol. 70, pp. 369-413.
Dancey, J., Eisenhauer, E. A., 1996, Br. J. Cancer, Vol. 74, pp. 327-338.
Dathe, M., Wieprecht, T., 1999, Bichim. Biophys. Acta, Vol. 1462, pp. 71-87.
Denny, W. A., Baguley, B. C., 2003, Curr. Top. Med. Chem., Vol. 3, pp. 339-353.
Destoumieux-Garzón, D., Peduzzi, J., Rebuffat, S., 2002, Biochimie, Vol. 84, pp. 511-519.
Diddens, H., Zahner, H., 1976, Eur. J. Biochem., Vol. 66, pp. 11-23.
Flatman, R. H., Howells, A. J., Heide, L., Fiedler, H. P., Maxwell, A., 2005, Antimicrob. Agents Chemother., Vol. 49, No. 3, p. 1093.
Hartwell, L. H., Weinert, T. A., 1989, Science, Vol. 246, pp. 629-634.
Heddle, J. G., Blance, S. J., Zamble, D. B., Hollfelder, F., Miller, D. A., Wentzell, L. M., Walsh, C. T. and Maxwell, A. (2001) “The antibiotic microcin B17 is a DNA gyrase poison: characterisation of the mode of inhibition.” J. Mol. Biol., Vol. 307, pp. 1223-1234.
Husain, I., Mohler, J. L., Seigler, H. F., Besterman, J. M., 1994, Cancer Res., Vol. 54, pp. 539-546.
Loffet, A., 2002, J. Pep. Sci., Vol. 8, pp. 1-7.
Park, C. B., Yi, K. S., Matsuzaki, K., Kim, M. S., Kim, S. C., 2000, Proc. Natl. Acad. Sci. USA., Vol. 97, pp. 8245-50
Peng, H, Marians, K. J., 1993, J. Biol. Chem., Vol. 268, pp. 24481-24490.
Pierrat, O. A. and A. Maxwell (2005). “Evidence for the role of DNA strand passage in the mechanism of action of microcin B17 on DNA gyrase.” Biochemistry, Vol. 44, No. 11, pp. 4204-4215.
Pierrat, O. A. and Maxwell, A. (2003) “The action of the bacterial toxin microcin B17: insight into the cleavage-religation reaction of DNA gyrase.” J. Biol. Chem., Vol. 278, pp. 35016-35023.
Pierrat, O. A., Maxwell, A., 2003, J. Biol. Chem., Vol. 278, pp. 35016-35023.
Pierrat, O. A., Maxwell, A., 2005, Biochemistry, Vol. 44, pp. 4204-4215.
Pommier, Y., Covey, J. M., Kerigan, D., Markovits, J. and Pharm, R. (1987), “DNA unwinding and inhibition of mouse-Leukemia-L1210 DNA topoisomerase I by intercalators.” Nucleic Acids Research, Vol. 15, pp. 6713-6730.
Potmesil, M., 1994, Cancer Res., Vol. 54, pp. 1431-1439.
Reece, R., Maxwell, A., 1989, J. Biol. Chem., Vol. 264, p. 19648.
Reece, R. J. and Maxwell, A. (1991) “DNA gyrase: structure and function.” CRC Crit. Rev. Biochem. Mol. Biol., Vol. 26, pp. 335-375.
Simmons, L. T., Andrianasolo, E., McPhail, K., Flatt, P., Gerwick, W. H., 2005, Mol. Cancer. Ther., Vol. 4, pp. 333-342.
Sinha Roy, R., Kelleher, N. L., Milne, J. C., Walsh, C. T., 1999, Chem. Biol., Vol. 6, pp. 305-318.
Sinha Roy, R. S., Kelleher, N. L., Milne, J. C. and Walsh, C. T. (1999), “In vivo processing and antibiotic activity of Microcin B17 analogs with varying ring content and altered bisheterocyclic sites.” Chem. Biol., Vol. 6, pp. 305-318.
Stanchev, M., Tabakova, S. Videnov, G., Golovinsky, E., Jung, G., 1999, Arch. Pharm., Vol. 332, pp. 297-304.
Stankova, I. G., Videnov, G., Golovinsky, E. V., Jung, G., 1999, J. Pept. Sci., Vol. 5, pp. 392-398.
Tushar K., Chakrahorty B., Krishna Mohan S., Kiran K. and Ajit C. K., “Nucleation of hairpin structure in a pyrrole amino acid containing peptide.” Tetrahedron Letters, Vol. 43, pp. 2589-2592.
Tushar K., Chakrahorty B., Krishna Mohan S., Kiran K. and Ajit C. K., “Development of 5-(aminomethyl)pyrrole-2-carboxylic acid as a constrained surrogate of Gly-Ala and its application in peptidomimetic studies.” Tetrahedron Letters, Vol. 44, pp. 471-473.
Videnov, G., D. Kaiser, et al. (1996). “Synthesis of Naturally Occurring, Conformationally Restricted Oxazole- and Thiazole-Containing Di- and Tripeptide Mimetics.” Angewandte Chemie, Vol. 35, No. 13/14, pp. 1503-1506.
Videnov, G., Kaiser, D., Kempter, C., Jung, G., 1996, Angewandte Chemie, Vol. 35, pp. 1503-1506.
Wall, M. K., L. A. Mitchenall, et al. (2004). “Arabidopsis thaliana DNA gyrase is targeted to chloroplasts and mitochondria.” Proc. Natl. Acad. Sci. USA, Vol. 101, No. 20, pp. 7821-7826.
Wall, M. K., Mitchenall, L. A., Maxwell, A., 2004, Proc. Natl. Acad. Sci. USA, Vol. 101, pp. 7821-7826.
Yorgey, P., Lee, J., Kordel, J., Vivas, E., Warner, P., Jebaratnam, D., Kolter, R., 1994, Proc. Natl. Acad. Sci. USA., Vol. 91, pp. 4519-4523.
Zasloff, M., 2002, Nature, Vol. 415, pp. 389-395.

1-125. (canceled)

126. A compound selected from compounds of the following formula and pharmaceutically acceptable salts, amides, esters, solvates, and hydrates thereof: 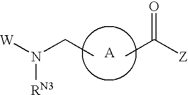
wherein:
W is independently a peptide group;
Z is independently a peptide group;
wherein each peptide group is a poly(amino acid) group and comprises two or more amino acids;
R^{N3} is independently: —H, C₁₋₆alkyl, C₂₋₆alkenyl, C₃₋₆cycloalkyl, or C₃₋₆cycloalkenyl, C₆₋₁₄-carboaryl, C₅₋₁₄heteroaryl, C₆₋₁₄-carboaryl-C₁₋₆alkyl, or C₅₋₁₄heteroaryl-C₁₋₆alkyl, and is optionally substituted;
the circle “A” denotes a five membered aromatic ring derived from pyrrole;
the group —C(═O)-Z is attached to a first ring atom of said five membered ring;
the group —CH₂—NR^{N3}—W is attached to a second ring atom of said five membered ring;
the A-ring is optionally additionally independently substituted.

127. A compound according to claim 126, selected from compounds of the following general formula and pharmaceutically acceptable salts, amides, esters, solvates, and hydrates thereof: 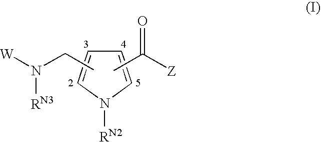
wherein:
W is independently a peptide group;
Z is independently a peptide group;
wherein each peptide group is a poly(amino acid) group and comprises two or more amino acids;
each of RN² and R^{N3} is independently: —H, C₁₋₆alkyl, C₂₋₆alkenyl, C₃₋₆cycloalkyl, or C₃₋₆cycloalkenyl, C₆₋₁₄carboaryl, C₅₋₁₄heteroaryl, C₆₋₁₄carboaryl-C₁₋₆alkyl, C₅₋₁₄heteroaryl-C₁₋₆alkyl, and is optionally substituted;
the group —CH₂—N(R^{N3})—W is independently attached at the 2-, 3-, 4-, or 5-ring position;
the group —C(═O)-Z is independently attached at one of the remaining ring positions;
the pyrrole ring is optionally additionally independently substituted.

128. A compound according to claim 126, wherein:
the group —CH₂—N(R^{N3})—W is independently attached at the 2- or 3-ring position; and
the group —C(═O)-Z is independently attached at the 4- or 5-ring position.

129. A compound according to claim 126, wherein:
the group —CH₂—N(R^{N3})—W is independently attached at the 2- or 3-ring position; and
the group —C(═O)-Z is independently attached at the 5-ring position;
and the compound is selected from compounds of the following general formulae and pharmaceutically acceptable salts, amides, esters, solvates, and hydrates thereof:

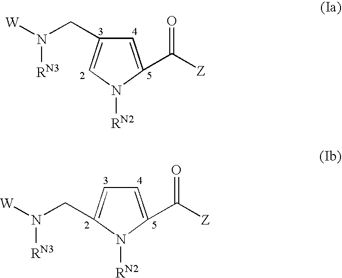

130. A compound according to claim 126, wherein:
the group —CH₂—N(R^{N3})—W is independently attached at the 2-ring position; and
the group —C(═O)-Z is independently attached at the 5-ring position.

131. A compound according to claim 126, wherein:
the group —CH₂—N(R^{N3})—W is independently attached at the 3-ring position; and
the group —C(═O)-Z is independently attached at the 5-ring position.

132. A compound according to claim 129, wherein RN² is independently —H or C₁₋₆alkyl.

133. A compound according to claim 129, wherein R^{N3} is independently —H or C₁₋₆alkyl.

134. A compound according to claim 132, wherein R^{N3} is independently —H or C₁₋₆alkyl.

135. A compound according to claim 134, wherein each poly(amino acid) group is selected from poly(amino acid) groups having from 2 to 10 amino acids.

136. A compound according to claim 134, wherein each poly(amino acid) group is selected from poly(amino acid) groups having from 2 to 5 amino acids.

137. A compound according to claim 134, wherein each poly(amino acid) group is selected from poly(amino acid) groups having 3, 4, or 5 amino acids.

138. A compound according to claim 134, wherein each poly(amino acid) group is selected from poly(amino acid) groups having 3 amino acids.

139. A compound according to claim 136, wherein each amino acid of said poly(amino acid) groups is a non-sterically hindered amino acid.

140. A compound according to claim 136, wherein each amino acid of said poly(amino acid) groups is selected from the naturally occurring α-amino acids.

141. A compound according to claim 136, wherein each amino acid of said poly(amino acid) groups is selected from glycine (Gly, G), alanine (Ala, A), and glutamine (Gln, Q).

142. A compound according to claim 134, wherein the group W—NR^{N3}—CH₂— is H-[AA¹]ₙ—NR^{N3}—CH₂—, wherein AA¹ is an amino acid group and n is an integer from 2 to 10.

143. A compound according to claim 134, wherein the group W—NR^{N3}—CH₂— is H-[AA¹]ₙ-NR^{N3}—CH₂—, wherein AA¹ is an amino acid group and n is an integer from 2 to 5.

144. A compound according to claim 134, wherein the group W—NR^{N3}—CH₂— is H-[AA¹]ₙ-NR^{N3}—CH₂—, wherein AA¹ is an amino acid group and n is 3, 4, or 5.

145. A compound according to claim 134, wherein the group W—NR^{N3}—CH₂— is H-[AA¹]ₙ-NR^{N3}—CH₂—, wherein AA¹ is an amino acid group and n is 3.

146. A compound according to claim 134, wherein the group —C(═O)-Z is C(═O)-[AA²]ₘ—OH, wherein AA² is an amino acid group and m is an integer from 2 to 10.

147. A compound according to claim 134, wherein the group —C(═O)-Z is C(═O)-[AA²]ₘ—OH, wherein AA² is an amino acid group and m is an integer from 2 to 5.

148. A compound according to claim 143, wherein the group —C(═O)-Z is C(═O)-[AA²]ₘ—OH, wherein AA² is an amino acid group and m is an integer from 2 to 5.

149. A compound according to claim 134, wherein the group —C(═O)-Z is C(═O)-[AA²]ₘ—OH, wherein AA² is an amino acid group and m is 3, 4, or 5.

150. A compound according to claim 134, wherein the group —C(═O)-Z is —C(═O)-[AA²]ₘ—OH, wherein AA² is an amino acid group and m is 3.

151. A compound according to claim 148, wherein each AA¹, if present, and each AA², if present, is independently selected from naturally occurring α-amino acids.

152. A compound according to claim 148, wherein each AA¹, if present, and each AA², if present, is independently selected from naturally occurring non-sterically hindered α-amino acids.

153. A compound according to claim 148, wherein each AA¹, if present, and each AA², if present, is independently selected from glycine (Gly, G), alanine (Ala, A), and glutamine (Gln, Q).

154. A compound according to claim 134, wherein the group W—NR^{N3}—CH₂— is H—[NH—CHR^{AA}—C(═O)]ₙ—NR^{N3}—CH₂—, wherein R^{AA} is an α-amino acid side-chain and n is an integer from 2 to 10.

155. A compound according to claim 134, wherein the group W—NR^{N3}—CH₂— is H—[NH—CHR^{AA}—C(═O)]ₙ—NR^{N3}—CH₂—, wherein R^{AA} is an α-amino acid side-chain and n is an integer from 2 to 5.

156. A compound according to claim 134, wherein the group W—NR^{N3}—CH₂— is H—[NH—CHR^{AA}—C(═O)]ₙ—NR^{N3}—CH₂— wherein R^{AA} is an α-amino acid side-chain and n is 3, 4, or 5.

157. A compound according to claim 134, wherein the group W—NR^{N3}—CH₂— is H—[NH—CHR^{AA}—C(═O)]ₙ—NR^{N3}—CH₂—, wherein R^{AA} is an α-amino acid side-chain and n is 3.

158. A compound according to claim 134, wherein the group —C(═O)-Z is C(═O)—[NH—CHR^{AA}—C(═O)]ₘ—OH, wherein R^{AA} is an α-amino acid side-chain and m is an integer from 2 to 10.

159. A compound according to claim 134, wherein the group —C(═O)-Z is —C(═O)—[NH—CHR^{AA}—C(═O)]ₘ—OH, wherein R^{AA} is an α-amino acid side-chain and m is an integer from 2 to 5.

160. A compound according to claim 155, wherein the group —C(═O)-Z is —C(═O)—[NH—CHR^{AA}—C(═O)]ₘ—OH, wherein R^{AA} is an α-amino acid side-chain and m is an integer from 2 to 5.

161. A compound according to claim 134, wherein the group —C(═O)-Z is —C(═O)—[NH—CHR^{AA}—C(═O)]ₘ—OH, wherein R^{AA} is an α-amino acid side-chain and m is 3, 4, or 5.

162. A compound according to claim 134, wherein the group —C(═O)-Z is C(═O)—[NH—CHR^{AA}—C(═O)]ₘ—OH, wherein R^{AA} is an α-amino acid side-chain and m is 3.

163. A compound according to claim 160, wherein each α-amino acid side-chain is independently selected from the α-amino acid side-chains of naturally occurring α-amino acids.

164. A compound according to claim 160, wherein each α-amino acid side-chain is independently selected from the α-amino acid side-chains of naturally occurring non-sterically hindered α-amino acids.

165. A compound according to claim 160, wherein each α-amino acid side-chain is independently selected from the α-amino acid side-chains of glycine (Gly, G), alanine (Ala, A), and glutamine (Gln, Q).

166. A compound according to claim 129, wherein W is -AGQ, and the group —CH₂—NR^{N3}—W is:

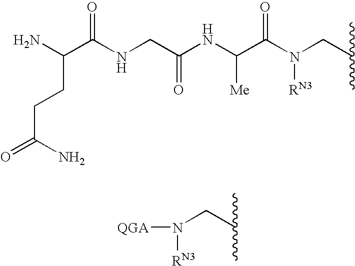

167. A compound according to claim 129, wherein Z is -GGA, and the group —C(═O)-Z is:

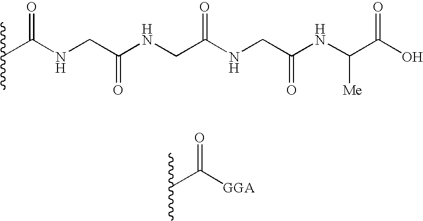

168. A compound according to claim 129, selected from the following compounds, and pharmaceutically acceptable salts, amides, esters, solvates, and hydrates thereof:

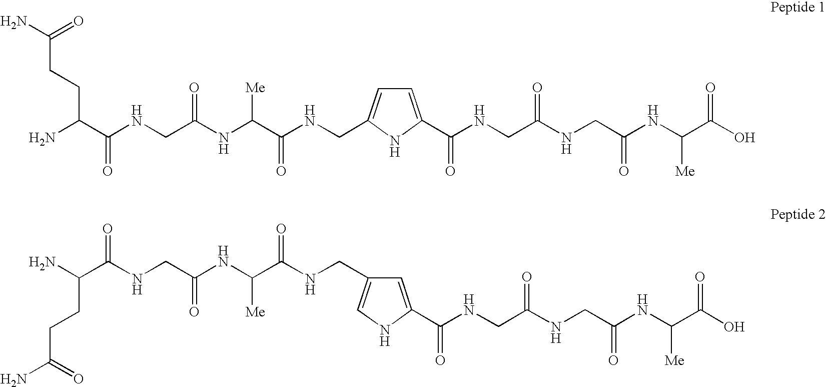

169. A composition comprising a compound according to claim 126 and a carrier or diluent.

170. A composition comprising a compound according to claim 126 and a pharmaceutically acceptable carrier or diluent.

171. A method of inhibiting DNA Gyrase activity in a cell, in vitro or in vivo, comprising contacting the cell with an effective amount of a compound according to claim 126.

172. A method of treatment of a disease or condition that is ameliorated by the inhibition of DNA Gyrase comprising administering to a patient in need of treatment a therapeutically effective amount of a compound according to claim 126.

173. A method of treatment of a bacterial infection comprising administering to a patient in need of treatment a therapeutically effective amount of a compound according to claim 126.

174. A method of treatment of cancer comprising administering to a patient in need of treatment a therapeutically effective amount of a compound according to claim 126.

175. A method of controlling plant growth comprising contacting a plant with an effective amount of a compound according to claim 126.

176. A method of controlling seedling growth comprising contacting a seedling with an effective amount of a compound according to claim 126.

177. A method of inhibiting germination of a seed, comprising contacting a seed with an effective amount of a compound according to claim 126.

178. A method of inhibiting germination of a sprouting seed, comprising contacting a sprouting seed with an effective amount of a compound according to claim 126.

179. A method of killing a microbe comprising contacting the microbe with an effective amount of a compound according to claim 126.

180. A method of killing bacteria comprising contacting the bacteria with an effective amount of a compound according to claim 126.
